(19) United States
(12) Patent Application Publication
(10) Pub. No.: US 2022/0363665 A1
(43) Pub. Date: Nov. 17, 2022

(54) QUINOLINE COMPOUNDS FOR THE TREATMENT OF AUTOIMMUNE DISEASE

(71) Applicant: Hoffmann-La Roche Inc., Little Falls, NJ (US)

(72) Inventors: Fabian DEY, Zurich (CH); Haixia LIU, Shanghai (CN); Hong SHEN, Shanghai (CN); Weixing ZHANG, Shanghai (CN); Zhiwei ZHANG, Shanghai (CN); Wei ZHU, Shanghai (CN)

(73) Assignee: Hoffmann-La Roche Inc., Little Falls, NJ (US)

(21) Appl. No.: 17/641,894

(22) PCT Filed: Sep. 9, 2020
(86) PCT No.: PCT/EP2020/075185

§ 371 Date: Mar. 10, 2022

(30) Foreign Application Priority Data
Sep. 10, 2019 (CN) PCT/CN2019/105117

(51) Int. Cl. C07D 401/10; C07D 401/12; C07D 215/38; C07D 498/08; C07D 471/08; C07D 487/10; A61P 37/06

(52) U.S. Cl. CPC C07D 401/10; C07D 401/12; C07D 215/38; C07D 498/08; C07D 471/08; C07D 487/10; A61P 37/06

(57) ABSTRACT

The present invention relates to compounds of formula (I), (I), wherein R¹, R² and R³ are as described herein, and their pharmaceutically acceptable salt, enantiomer or diastereomer thereof, and compositions including the compounds and methods of using the compounds.

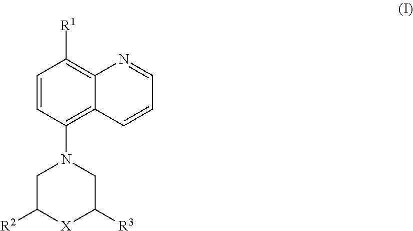

The present invention relates to organic compounds useful for therapy and/or prophylaxis in a mammal, and in particular to antagonist of TLR7 and/or TLR8 and/or TLR9 useful for treating systemic lupus erythematosus or lupus nephritis.

### FIELD OF THE INVENTION

Autoimmune connective tissue disease (CTD) include prototypical autoimmune syndromes such as Systemic Lupus Erythematosus (SLE), primary Sjögren's syndrome (pSjS), mixed connective tissue disease (MCTD), Dermatomyositis/Polymyositis (DM/PM), Rheumatoid Arthritis (RA), and systemic sclerosis (SSc). With the exception of RA, no really effective and safe therapies are available to patients. SLE represents the prototypical CTD with a prevalence of 20-150 per 100,000 and causes broad inflammation and tissue damage in distinct organs, from commonly observed symptoms in the skin and joints to renal, lung, or heart failure. Traditionally, SLE has been treated with nonspecific anti-inflammatory or immunosuppressive drugs. However, long term usage of immunosuppressive drug, e.g. corticosteroids is only partially effective, and is associated with undesirable toxicity and side effects. Belimumab is the only FDA-approved drug for lupus in the last 50 years, despite its modest and delayed efficacy in only a fraction of SLE patients (Navarra, S. V. et al Lancet 2011, 377, 721.). Other biologics, such as anti-CD20 mAbs, mAbs against or soluble receptors of specific cytokines, have failed in most clinical studies. Thus, novel therapies are required that provide sustained improvement in a greater proportion of patient groups and are safer for chronic use in many autoimmune as well as auto-inflammation diseases.

Toll Like Receptors (TLR) are an important family of pattern recognition receptors (PRR) which can initiate broad immune responses in a wide variety of immune cells. As natural host defense sensors, endosomal TLRs 7, 8 and 9 recognize nucleic acids derived from viruses, bacteria; specifically, TLR7/8 and TLR9 recognize single-stranded RNA (ssRNA) and single-stranded CpG-DNA, respectively. However, aberrant nucleic acid sensing of TRL7,8,9 is considered as a key node in a broad of autoimmune and auto-inflammatory diseases (Krieg, A. M. et al. Immunol. Rev. 2007, 220, 251. Jiménez-Dalmaroni, M. J. et al Autoimmun Rev. 2016, 15, 1. Chen, J. Q., et al. Clinical Reviews in Allergy & Immunology 2016, 50, 1.) Therefore, TLR7,8,9 represents a new therapeutic target for autoimmune and auto-inflammatory diseases, for which no effective steroid-free and non-cytotoxic oral drugs exist, and inhibition of these pathways from the very upstream may deliver satisfying therapeutic effects. From a safety perspective, because there are multiple nucleic acid sensing pathways (e.g. other TLRs, cGAS/STING), such redundancy should still allow responses to infection in the presence of TLR789 inhibition. As such, we proposed and invented oral compounds that target and suppress TLR7,8,9 for the treatment of autoimmune and auto-inflammatory diseases.

### SUMMARY OF THE INVENTION

The present invention relates to novel compounds of formula (I),

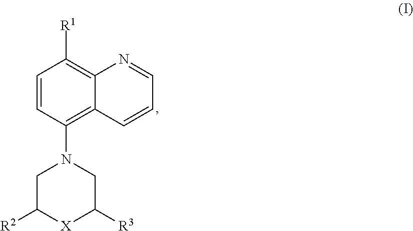

Wherein

R¹ is halogen, C₁₋₆alkyl, haloC₁₋₆alkyl or C₂₋₆alkynyl;
R² is amino or —CONR⁴R⁵; wherein

R⁴ is H;

R⁵ is aminoC₁₋₆alkyl, heterocyclyl or heterocyclylC₁₋₆alkyl;

or R⁴ and R⁵ together with the nitrogen they are attached to form a heterocyclyl;

R³ is C₁₋₆alkyl;

### X is O or CH₂;

or a pharmaceutically acceptable salt, enantiomer or diastereomer thereof.

Another object of the present invention is related to novel compounds of formula (I), their manufacture, medicaments based on a compound in accordance with the invention and their production as well as the use of compounds of formula (I) as TLR7 and/or TLR8 and/or TLR9 antagonist, and for the treatment or prophylaxis of systemic lupus erythematosus or lupus nephritis. The compounds of formula (I) show superior TLR7 and/or TLR8 and/or TLR9 antagonism activity. In addition, the compounds of formula (I) also show good solubility, human microsome stability and SDPK profiles, as well as low CYP inhibition.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₂₋₆alkynyl” denotes a saturated, linear or branched chain alkynyl group containing 1 to 6, particularly 1 to 4 carbon atoms, for example ethynyl, propynyl and the like. Particular “C₁₋₆alkyl” group is ethynyl.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group has been replaced by same or different halogen atoms, particularly fluoro atoms. Examples of haloC₁₋₆alkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, difluoromethyl, trifluoromethyl and trifluoroethyl.

The term “halopiperidinyl” denotes a piperidinyl group wherein at least one of the hydrogen atoms of the piperidinyl group has been replaced by same or different halogen atoms, particularly fluoro atoms. Examples of halopiperidinyl include fluoropyrrolidinyl and difluoropiperidinyl.

The term “halopyrrolidinyl” denotes a pyrrolidinyl group wherein at least one of the hydrogen atoms of the pyrrolidinyl group has been replaced by same or different halogen atoms, particularly fluoro atoms. Examples of halopiperidinyl include fluoropyrrolidinyl and difluoropyrrolidinyl.

The term “heterocyclyl” denotes a monovalent saturated or partly unsaturated mono- or bicyclic ring system of 3 to 12 ring atoms, comprising 1, 2, or 3 ring heteroatoms selected from N, O and S, the remaining ring atoms being carbon. In particular embodiments, heterocyclyl is a monovalent saturated monocyclic ring system of 4 to 10 ring atoms, comprising 1, 2, or 3 ring heteroatoms selected from N, O and S, the remaining ring atoms being carbon. Examples for monocyclic saturated heterocyclyl are aziridinyl, oxiranyl, azetidinyl, oxetanyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, oxazepanyl, diazepanyl, homopiperazinyl, or oxazepanyl. Examples for bicyclic saturated heterocyclyl are azabicyclo[3.2.1]octyl, quinuclidinyl, oxaazabicyclo[3.2.1]octyl, azabicyclo[3.3.1]nonanyl, oxaazabicyclo[3.3.1]nonyl, thiaazabicyclo[3.3.1]nonyl, oxaazabicyclo[2.2.2]heptanyl, 1,2,3,3a,4,5,6,6a-octahydropyrrolo[3,4-c]pyrrolyl, 2,7-diazaspiro[4.4]nonanyl, 1,3,4,6,7, 8,9,9a-octahydropyrazino[2,1-c][1,4]oxazinyl, azaspiro[2.4]heptanyl, azabicyclo[3.2.1]octanyl, diazaspiro[5.5]undecanyl, oxaazabicyclo[3.3.1]nonanyl, azabicyclo[2.2.1]heptanyl and azabicyclo[3.2.0]heptanyl. Examples for partly unsaturated heterocyclyl are dihydrofuryl, imidazolinyl, dihydro-oxazolyl, tetrahydropyridinyl, and dihydropyranyl. Monocyclic or bicyclic heterocyclyl can be further substituted by halogen, hydroxy, amino, C₁₋₆alkyl or haloC₁₋₆alkyl.

The term “enantiomer” denotes two stereoisomers of a compound which are non-superimposable mirror images of one another.

The term “diastereomer” denotes a stereoisomer with two or more centers of chirality and whose molecules are not mirror images of one another. Diastereomers have different physical properties, e.g. melting points, boiling points, spectral properties, and reactivities.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

### Antagonist of Tlr7 and/Oo Tlr8 and/or Tlr9

The present invention relates to a compound of formula (I),

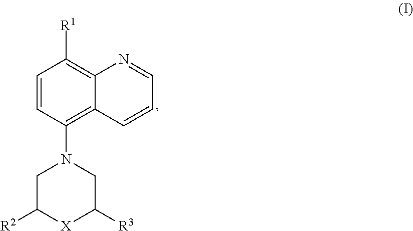

wherein

R¹ is halogen, C₁₋₆alkyl, halo₁₋₆alkyl or C₂₋₆alkynyl;
R² is amino or —CONR⁴R⁵; wherein

R⁴ is H;

R⁵ is aminoC₁₋₆alkyl, heterocyclyl or heterocyclylC₁₋₆alkyl;

or R⁴ and R⁵ together with the nitrogen they are attached to form a heterocyclyl;

R³ is C₁₋₆alkyl;

### X is O or CH₂;

or a pharmaceutically acceptable salt, enantiomer or diastereomer thereof.

A further embodiment of present invention is (ii) a compound of formula (I), wherein

R¹ is halogen, C₁₋₆alkyl, halo₁₋₆alkyl or C₂₋₆alkynyl;
R² is amino or —CONR⁴R⁵; wherein

R⁴ is H;

R⁵ is (C₁₋₆alkylmorpholinyl)C₁₋₆alkyl, (C₁₋₆alkylpiperidinyl)C₁₋₆alkyl, aminoC₁₋₆alkyl, azabicyclo[2.2.1]heptanyl, azabicyclo[3.2.0]heptanyl, azabicyclo[3.2.1]octanyl, azabicyclo[3.3.1]nonanyl, azaspiro[2.4]heptanyl substituted by C₁₋₆alkyl, azepanyl, C₁₋₆alkylpiperidinyl, halopiperidinyl, halopyrrolidinyl, halopyrrolidinylC₁₋₆alkyl, morpholinylC₁₋₆alkyl, oxaazabicyclo[3.3.1]nonanyl or oxazepanyl;

or R⁴ and R⁵ together with the nitrogen they are attached to form diazaspiro[5.5]undecanyl, diazaspiro[4.4]nonanyl, azetidinyl, piperidinyl or pyrrolidinyl, said azetidinyl, piperidinyl and pyrrolidinyl being substituted by amino;

R³ is C₁₋₆alkyl;

### X is O or CH₂;

or pharmaceutically acceptable salt, enantiomer or diastereomer thereof.

A further embodiment of present invention is (iii) a compound of formula (I) according to (ii), wherein R¹ is Br, Cl, I, CF₃, ethynyl or methyl.

A further embodiment of present invention is (iv) a compound of formula (I) according to (iii), wherein R^{I} is Cl or CF₃.

A further embodiment of present invention is (v) a compound of formula (I) according to any one of (i) to (iv), wherein R² is —CONR⁴R⁵, wherein R⁴ is H; R⁵ is (C₁₋₆alkylmorpholinyl)C₁₋₆alkyl, (C₁₋₆alkylpiperidinyl)C₁₋₆alkyl, azabicyclo[3.2.1]octanyl, azabicyclo[3.3.1]nonanyl, azepanyl, C₁₋₆alkylpiperidinyl, morpholinylC₁₋₆alkyl or oxaazabicyclo[3.3.1]nonanyl.

A further embodiment of present invention is (vi) a compound of formula (I) according to any one of (i) to (v), wherein R² is —CONR⁴R⁵, wherein R⁴ is H; R⁵ is (methylmorpholinyl)methyl, (methylpiperidinyl)methyl, 3-azabicyclo[3.2.1]octan-8-yl, 8-azabicyclo[3.2.1]octan-3-yl, 9-azabicyclo[3.3.1]nonan-3-yl, 3-azabicyclo[3.3.1]nonan-7-yl, 3-azabicyclo[3.3.1]nonan-9-yl, azepan-4-yl, methylpiperidinyl, morpholinylmethyl, 3-oxa-7-azabicyclo[3.3.1]nonan-9-yl or 3-oxa-9-azabicyclo[3.3.1]nonan-7-yl.

A further embodiment of present invention is (vii) a compound of formula (I) according to any one of (i) to (vi), wherein R⁵ is azabicyclo[3.2.1]octanyl or azabicyclo[3.3.1]nonanyl.

A further embodiment of present invention is (viii) a compound of formula (I) according to any one of (i) to (vii), wherein R⁵ is 3-azabicyclo[3.2.1]octan-8-yl, 8-azabicyclo[3.2.1]octan-3-yl, 9-azabicyclo[3.3.1]nonan-3-yl, 3-azabicyclo[3.3.1]nonan-7-yl or 3-azabicyclo[3.3.1]nonan-9-yl.

A further embodiment of present invention is (ix) a compound of formula (I) according to any one of (i) to (viii), wherein X is O.

Another embodiment of present invention is that (x) particular compounds of formula (I) are the following:

(3R,5S)-5-methyl-1[8-(trifluoromethyl)-5-quinolyl]piperidin-3-amine;

cis-(2R,6R)-N-(4-fluoropyrrolidin-3-yl)-6-methyl-4-[8-(trifluoromethyl)-5-quinolyl]morpholine-2-carboxamide;

(2R,6R)-N-[(3R,4S)-4-fluoropyrrolidin-3-yl]-6-methyl-4-[8-(trifluoromethyl)-5-quinolyl]morpholine-2-carboxamide;

(2R,6R)-N-[(3S,4R)-4-fluoropyrrolidin-3-yl]-6-methyl-4-[8-(trifluoromethyl)-5-quinolyl]morpholine-2-carboxamide;

(2R,6R)-6-methyl-N-[(4-methylmorpholin-2-yl)methyl]-4-[8-(trifluoromethyl)-5-quinolyl]morpholine-2-carboxamide;

(2R,6R)-6-methyl-N-[(1-methyl-2-piperidypmethyl]-4-[8-(trifluoromethyl)-5-quinolyl]morpholine-2-carboxamide;

(2R,6R)-6-methyl-N-(1-methyl-4-piperidyl)-4-[8-(trifluoromethyl)-5-quinolyl]morpholine-2-carboxamide;

(2R,6R)-N-(2-amino-2-methyl-propyl)-6-methyl-4-[8-(trifluoromethyl)-5-quinolyl] morpholine-2-carboxamide;

(3-aminoazetidin-1-yl)-[R2R,6R)-6-methyl-4-[8-(trifluoromethyl)-5-quinolyl] morpholin-2-yl]methanone; (2R,6R)-N-(azepan-4-yl)-6-methyl-4-[8-(trifluoromethyl)-5-quinolyl]morpholine-2-carboxamide;

(2R,6R)-6-methyl-N-(5-methyl-5-azaspiro[2.4]heptan-7-yl)-4-[8-(trifluoromethyl)-5-quinolyl]morpholine-2-carboxamide;

(3-aminopyrrolidin-1-yl)-[(R2R,6R)-6-methyl-4-[8-(trifluoromethyl)-5-quinolyl]morpholin-2-yl]methanone;

(2R,6R)-6-methyl-N-[(4-methylmorpholin-3-yl)methyl]-4-[8-(trifluoromethyl)-5-quinolyl]morpholine-2-carboxamide;

(4-amino-1-piperidyl)-[(2R,6R)-6-methyl-4-[8-(trifluoromethyl)-5-quinolyl]morpholin-2-yl]methanone; (2R,6R)-6-methyl-N-(2-morpholinoethyl)-4-[8-(trifluoromethyl)-5-quinolyl]morpholine-2-carboxamide;

(2R,6R)-6-methyl-N-(1,4-oxazepan-6-yl)-4-[8-(trifluoromethyl)-5-quinolyl]morpholine-2-carboxamide;

(3R,5S)-5-methyl-1-(8-methyl-5-quinolyppiperidin-3-amine; (2R,6R)-6-methyl-N-(1-methyl-4-piperidyl)-4-(8-methyl-5-quinolyl)morpholine-2-carboxamide;

(2R,6R)-6-methyl-N-[(4-methylmorpholin-2-yl)methyl]-4-(8-methyl-5-quinolyl)morpholine-2-carboxamide;

(2R,6R)-6-methyl-N-[(4-methylmorpholin-2-yl)methyl]-4-(8-nitro-5-quinolypmorpholine-2-carboxamide;

cis-(2R,6R)-N-[4-4-fluoropyrrolidin-3-yl]-6-methyl-4-(8-nitro-5-quinolyl)morpholine-2-carboxamide;

(2R,6R)-6-methyl-N-(1-methyl-4-piperidyl)-4-(8-nitro-5-quinolyl)morpholine-2-carboxamide;

cis-(2R,6R)-N-[4-fluoropyrrolidin-3-yl]-6-methyl-4-(8-methyl-5-quinolyl)morpholine-2-carboxamide;

(2R,6R)-4-(8-chloro-5-quinolyl)-N-[(3S,4R)-4-fluoropyrrolidin-3-yl]-6-methyl-morpholine-2-carboxamide;

(3R,5S)-1-(8-chloro-5-quinolyl)-5-methyl-piperidin-3-amine;

(2R,6R)-4-(8-chloro-5-quinolyl)-6-methyl-N-(1-methyl-4-piperidyl)morpholine-2-carboxamide;

(2R,6R)-N-(azepan-4-yl)-4-(8-chloro-5-quinolyl)-6-methyl-morpholine-2-carboxamide;

(2R,6R)-4-(8-chloro-5-quinolyl)-6-methyl-N-[(4-methylmorpholin-2-yl)methyl]morpholine-2-carboxamide;

(2R,6R)-N-[[(2S,4R)-4-fluoropyrrolidin-2-yl]methyl]-6-methyl-4-[8-(trifluoromethyl)-5-quinolyl]morpholine-2-carboxamide;

(2R,6R)-N-(5,5-difluoro-3-piperidyl)-6-methyl-4-[8-(trifluoromethyl)-5-quinolyl] morpholine-2-carboxamide;

(2R,6R)-N-[(4,4-difluoropyrrolidin-3-yl)methyl]-6-methyl-4-[8-(trifluoromethyl)-5-quinolyl] morpholine-2-carboxamide;

(2R,6R)-N-[[(2S)-4,4-difluoropyrrolidin-2-yl]methyl]-6-methyl-4-[8-(trifluoromethyl)-5-quinolyl]morpholine-2-carboxamide;

(2R,6R)-N-(3-azabicyclo[3.2.1]octan-8-yl)-6-methyl-4-[8-(trifluoromethyl)-5-quinolyl] morpholine-2-carboxamide;

3,9-diazaspiro[5.5]undecan-3-yl-[(R2R,6R)-6-methyl-4-[8-(trifluoromethyl)-5-quinolyl]morpholin-2-yl]methanone;

(2R,6R)-6-methyl-N-(3-oxa-9-azabicyclo [3.3.1]nonan-7-yl)-4-[8-(trifluoromethyl)-5-quinolyl]morpholine-2-carboxamide;

(2R,6R)-N-(9-azabicyclo[3.3.1]nonan-3-yl)-6-methyl-4-[8-(trifluoromethyl)-5-quinolyl]morpholine-2-carboxamide;

(2R,6R)-6-methyl-N-[1-(4-methylmorpholin-2-yl)ethyl]-4-[8-(trifluoromethyl)-5-quinolyl]morpholine-2-carboxamide;

2,7-diazaspiro [4.4]nonan-2-yl-[R2R,6R)-6-methyl-4-[8-(trifluoromethyl)-5-quinolyl]morpholin-2-yl]methanone;

(2R,6R)-N-(3-azabicyclo [3.3.]nonan-7-yl)-6-methyl-4-[8-(trifluoromethyl)-5-quinolyl]morpholine-2-carboxamide;

(2R,6R)-4-(8-iodo-5-quinolyl)-6-methyl-N-(1-methyl-4-piperidyl)morpholine-2-carboxamide;

(2R,6R)-4-(8-iodo-5-quinolyl)-6-methyl-N-[(4-methylmorpholin-2-yl)methyl]morpholine-2-carboxamide;

cis-(2R,6R)-N-[4-fluoropyrrolidin-3-yl]-4-(8-iodo-5-quinolyl)-6-methyl-morpholine-2-carboxamide;

cis-(2R,6R)-4-(8-bromo-5-quinolyl)-N-[4-fluoropyrrolidin-3-yl]-6-methyl-morpholine-2-carboxamide;

(2R,6R)-4-(8-bromo-5-quinolyl)-6-methyl-N-(1-methyl-4-piperidyl)morpholine-2-carboxamide;

(2R,6R)-4-(8-ethynyl-5-quinolyl)-6-methyl-N-(1-methyl-4-piperidyl)morpholine-2-carboxamide;

(2R,6R)-N-[1R,4R)-2-azabicyclo[2.2.1]heptan-5-yl]-6-methyl-4-[8-(trifluoromethyl)-5-quinolyl]morpholine-2-carboxamide;

(2R,6R)-N-(3-azabicyclo[3.3.1]nonan-9-yl)-6-methyl-4-[8-(trifluoromethyl)-5-quinolyl]morpholine-2-carboxamide;

(2R,6R)-6-methyl-N-(3-oxa-7-azabicyclo[3.3.1]nonan-9-yl)-4-[8-(trifluoromethyl)-5-quinolyl]morpholine-2-carboxamide;

(2R,6R)-N-(8-azabicyclo[3.2.0]octan-3-yl)-6-methyl-4-[8-(trifluoromethyl)-5-quinolyl]morpholine-2-carboxamide;

(2R,6R)-6-methyl-N-(morpholin-2-ylmethyl)-4-[8-(trifluoromethyl)-5-quinolyl]morpholine-2-carboxamide; and

(2R,6R)-N-(3-azabicyclo[3.2.0]heptan-6-yl)-6-methyl-4-[8-(trifluoromethyl)-5-quinolyl]morpholine-2-carboxamide;

or a pharmaceutically acceptable salt, enantiomer or diastereomer thereof.

### Synthesis

The compounds of the present invention can be prepared by any conventional means. Suitable processes for synthesizing these compounds as well as their starting materials are provided in the schemes below and in the examples. All substituents, in particular, R¹ to R⁵ are as defined above unless otherwise indicated. Furthermore, and unless explicitly otherwise stated, all reactions, reaction conditions, abbreviations and symbols have the meanings well known to a person of ordinary skill in organic chemistry.

A general synthetic route for preparing the compound of formula (I) is shown in Scheme 1 below.

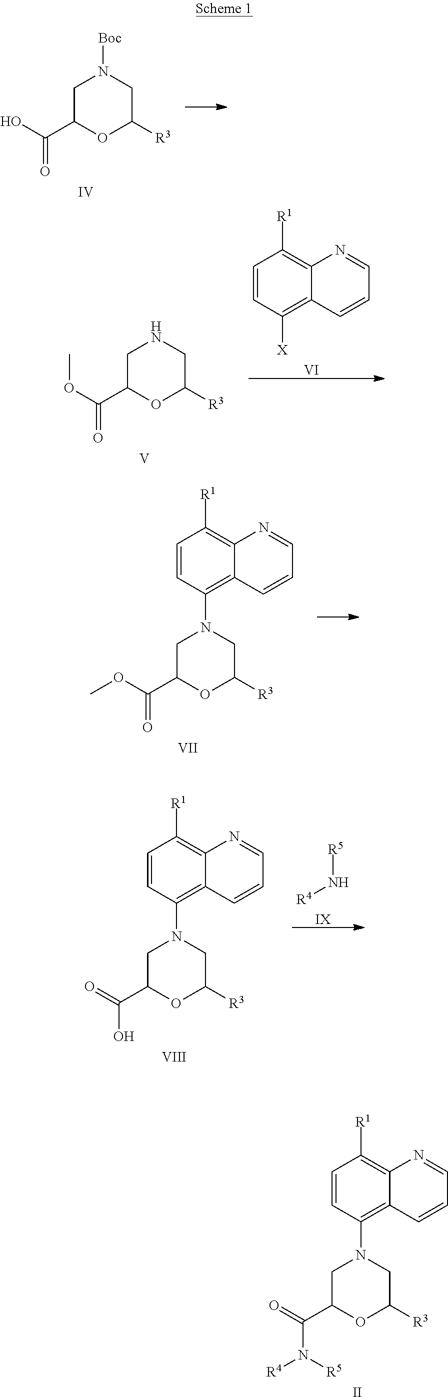

The synthesis of the compound of formula (II) is shown in Scheme 1. The starting material carboxylic acid (IV) can be converted to ester (V) via esterification with MeOH. The subsequent coupling of halide (VI) with compound of formula (V) can be achieved by direct coupling in the presence of a base, such as DIPEA and K₂CO₃, or under Buchwald-Hartwig amination conditions (ref: Acc. Chem. Res. 1998, 31, 805-818; Chem. Rev. 2016, 116, 12564-12649; Topics in Current Chemistry, 2002, 219, 131-209; and references cited therein) with a catalyst, such as Ruphos Pd-G2, and a base, such as Cs₂CO₃, to provide compound of formula (VII). Hydrolysis of compound of formula (VII) in basic condition, such as LiOH in THF/water, gives carboxylic acid (VIII), which is condensed with amine (IX) in the presence of a coupling reagent, such as HATU, to give the compound of formula (II). In some embodiment, the coupling of compound of formula (VIII) and amine (IX) may give a product containing a protecting group, e.g. Boc, originated from amine (IX), which will be removed before affording the final compound of formula (II).

Alternatively, the compound of formula (II) can be prepared as shown in Scheme 2,

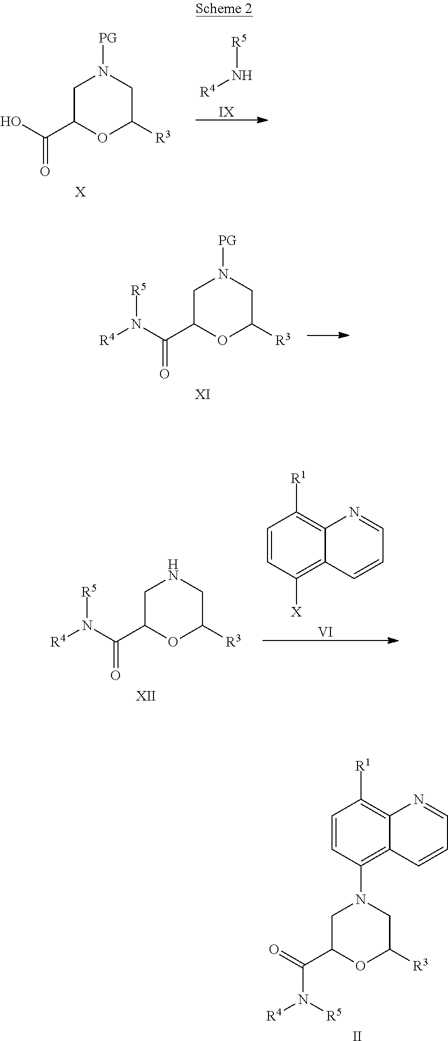

wherein PG is protecting group, such as Boc and Cbz.

The carboxylic acid (X) can be condensed with amine (IX) in the presence of a coupling reagent, such as HATU, to give compound of formula (XI). The protecting group of compound of formula (XI), e.g. Boc or Cbz, can be removed under acidic condition, such as TFA/CH₂Cl₂ and HCl in dioxane, or under hydrogenation condition (e.g. Pd-C, H₂) to give compound of formula (XII). Coupling of compound of formula (XII) with the halide (VI) under the Buchwald-Hartwig amination condition with a catalyst, such as Ruphos Pd-G2, and a base, such as Cs₂CO₃, affords the compounds of formula (II). In some embodiment, the coupling of halide (V) and compound of formula (XII) may give a product containing a protecting group, e.g. Boc, originated from amine (IX), which will be removed before affording the final compound of formula (II).

A general synthetic route for preparing the compounds of formula (III) is shown in Scheme 3.

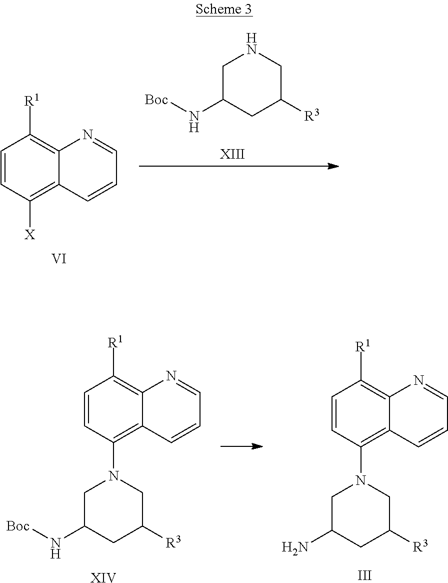

The synthesis of the compound of formula (III) can be achieved by the coupling of halide (VI) with amine (XIII) in the presence of a base, such as DIPEA and K₂CO₃, or under Buchwald-Hartwig amination conditions with a catalyst, such as Ruphos Pd-G2, and a base, such as Cs₂CO₃, to give compound of formula (XIV). Compound of formula (XIV) is deprotected under an acidic condition, such as TFA/CH₂Cl₂ and HCl in dioxane, to afford the final compound of formula (III).

This invention also relates to a process for the preparation of a compound of formula (I) comprising any of the following steps:

a) the reaction of compound of formula (VIII),

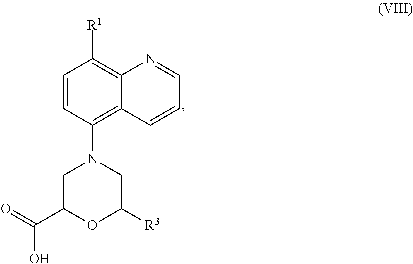

with amine (IX) in the presence of a coupling reagent;

b) the reaction of compound of formula (XII),

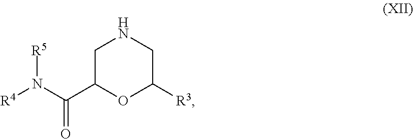

with compound of formula (VI) in the presence of a catalyst and a base;

c) the reaction of compound of formula (XIV),

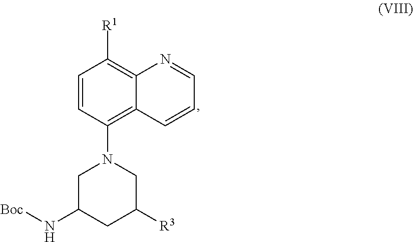

in the presence of an acid;
wherein R¹, R³, R⁴ and R⁵ are defined above.

In step a), the coupling reagent can be for example HATU.

In step b), the catalyst can be for example Ruphos Pd-G2, the base can be for example Cs₂CO₃.

In step c), the acid can be for example TFA/CH₂Cl₂ and HCl in dioxane.

A compound of formula (I), (II) or (III) when manufactured according to the above process is also an object of the invention.

Compounds of this invention can be obtained as mixtures of diastereomers or enantiomers, which can be separated by methods well known in the art, e.g. (chiral) HPLC or SFC.

### Indications and Methods of Treatment

The present invention provides compounds that can be used as TLR7 and/or TLR8 and/or TLR9 antagonist, which inhibits pathway activation through TLR7 and/or TLR8 and/or TLR9 as well as respective downstream biological events including, but not limited to, innate and adaptive immune responses mediated through the production of all types of cytokines and all forms of auto-antibodies. Accordingly, the compounds of the invention are useful for blocking TLR7 and/or TLR8 and/or TLR9 in all types of cells that express such receptor(s) including, but not limited to, plasmacytoid dendritic cell, B cell, T cell, macrophage, monocyte, neutrophil, keratinocyte, epithelial cell. As such, the compounds can be used as a therapeutic or prophylactic agent for systemic lupus erythematosus and lupus nephritis.

The present invention provides methods for treatment or prophylaxis of systemic lupus erythematosus and lupus nephritis in a patient in need thereof.

Another embodiment includes a method of treating or preventing systemic lupus erythematosus and lupus nephritis in a mammal in need of such treatment, wherein the method comprises administering to said mammal a therapeutically effective amount of a compound of formula (I), a stereoisomer, tautomer, prodrug or pharmaceutically acceptable salt thereof.

### EXAMPLES

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:

ACN: acetonitrile

Boc₂O: di-tert-butyl dicarbonate

Cb₂Cl: benzylchloroformate

DCE: dichloroethane

DIPEA or DIEA: N,N-diisopropylethylamine

DMAP: 4-dimethylaminopyridine

EA or EtOAc: ethyl acetate

FA: formic acid

HATU: 1-[Bis(dimethylamino)methylene]-1H-1,2,3-triazolo[4,5-b]pyridinium 3-oxid hexafluorophosphate

IC₅₀: half inhibition concentration

IPA: isopropanol

LCMS liquid chromatography-mass spectrometry

L-DATA: Di-p-anisoyl-L-tartaric acid

MS: mass spectrometry

MTBE: methyl tert-butyl ether

NBS: N-bromosuccinimide

NIS: N-iodosuccinimide

PE: petroleum ether

prep-HPLC: preparative high performance liquid chromatography

prep-TLC: preparative thin layer chromatography

rt: room temperature

RT: retention time

RuPhos Pd G2: chloro(2-dicyclohexylphosphino-2′,6′-diisopropoxy-1,1′-biphenyl)[2-(2′-amino-1,1′-biphenyl)]palladium (II) 2nd generation

SFC: supercritical fluid chromatography

TFA: trifluoroacetic acid

TLC: thin layer chromatography

v/v volume ratio

DDI drug-drug-interaction

LYSA lyophilisation solubility assay

FILM human liver microsome

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25>150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃.H₂O in MeOH), back pressure 100bar, detection UV @ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):

Acidic condition I: A: 0.1% TFA in H₂O; B: 0.1% TFA in acetonitrile;

Acidic condition II: A: 0.0375% TFA in H₂O; B: 0.01875% TFA in acetonitrile;

Basic condition I: A: 0.1% NH₃.H₂O in H₂O; B: acetonitrile;

Basic condition II: A: 0.025% NH₃.H₂O in H₂O; B: acetonitrile;

Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Example 1

(3R,5S)-5-methyl-1-[8-(trifluoromethyl)-5-quinolyl]piperidin-3-amine

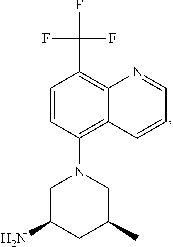 [SMILES: C[C@H]1C[C@@H](N)CN(c2ccc(C(F)(F)F)c3ncccc23)C1]

The title compound was prepared according to the following scheme:

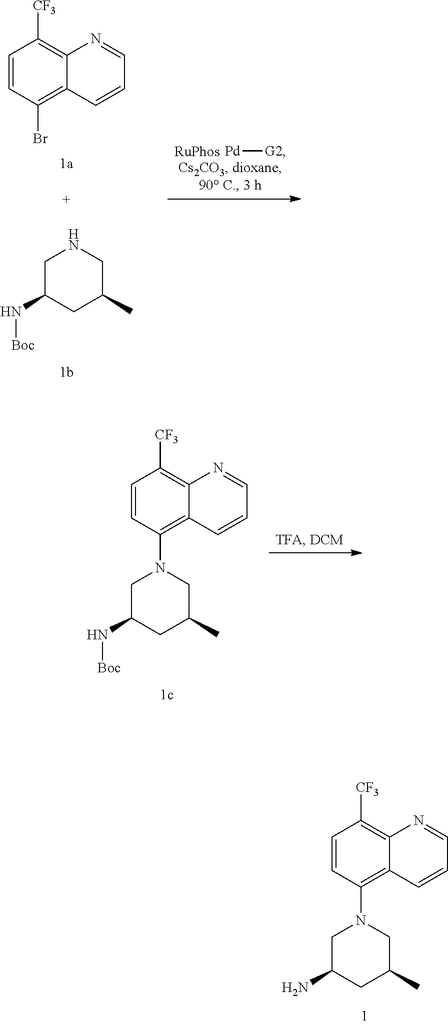

Step 1: tert-butyl N-[3R,55)-5-methyl-1-[8-(trifluoromethyl)-5-quinolyl]-3-piperidyl]carbamate (Compound 1c)

To a solution of 5-bromo-8-(trifluoromethyl)quinoline (compound 1a, 50 mg, 0.18 mmol), tert-butyl N-[(3R,5S)-5-methyl-3-piperidyl]carbamate (Reference: WO 2015057655 A1) (compound 1b, 47 mg, 0.22 mmol) and Cs₂CO₃ (118 mg, 0.36 mmol) in 1,4-dioxane (2 mL) was added RuPhos Pd G2 (CAS: 1375325-68-0, 14 mg, 0.020 mmol) at 25° C. under N₂. The reaction mixture was heated at 90° C. for 4h, then cooled to rt and concentrated to give a crude product which was purified by prep-TLC (DCM/MeOH=20/1) to give compound 1c (45 mg) as a yellow oil. MS: calc'd 410 (MH⁺), measured 410 (MH⁺).

Step 2: Preparation of (3R,5S)-5-methyl-1-[8-(trifluoromethyl)-5-quinolyl]piperidin-3-amine (Example 1)

To a solution of tert-butyl N-[3R,5S)-5-methyl-1-[8-(trifluoromethyl)-5-quinolyl]-3-piperidyl]carbamate (compound 1c, 45 mg, 0.11 mmol) in DCM (2 mL) was added TFA (0.5 mL) at 0° C. The reaction mixture was stirred at rt for 2h, then concentrated. The residue was purified by prep-HPLC to give Example 1 (37 mg) as a yellow solid. MS: calc'd 310 (MH⁺), measured 310 (MH⁺). ¹ NMR (400 MHz, METHANOL-d₄) δ =8.98 (dd, J=1.6, 4.1 Hz, 1H), 8.62 (dd, J=1.6, 8.7 Hz, 1H), 8.09 (d, J=8.0 Hz, 1H), 7.65 (dd, J=4.3, 8.7 Hz, 1H), 7.31 (d, J=8.0 Hz, 1H), 3.75−3.64 (m, 2H), 3.43 (d, J=9.2 Hz, 1H), 2.85−2.74 (m, 1H), 2.51 (t, J=11.4 Hz, 1H), 2.37−2.18 (m, 2H), 1.30−1.22 (m, 1H), 1.09 (d, J=6.7 Hz, 3H).

### Example 2

cis-(2R,6R)-N-(4-fluoropyrrolidin-3-yl)-6-methyl-4-[8-(trifluoromethyl)-5-quinolyl]morpholine-2-carboxamide (mixture of two cis diastereoisomers at the position marked with *)

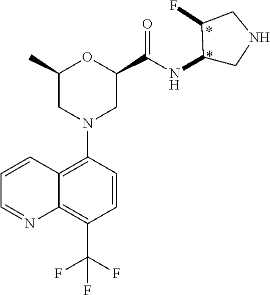

The title compound was prepared according to the scheme below:

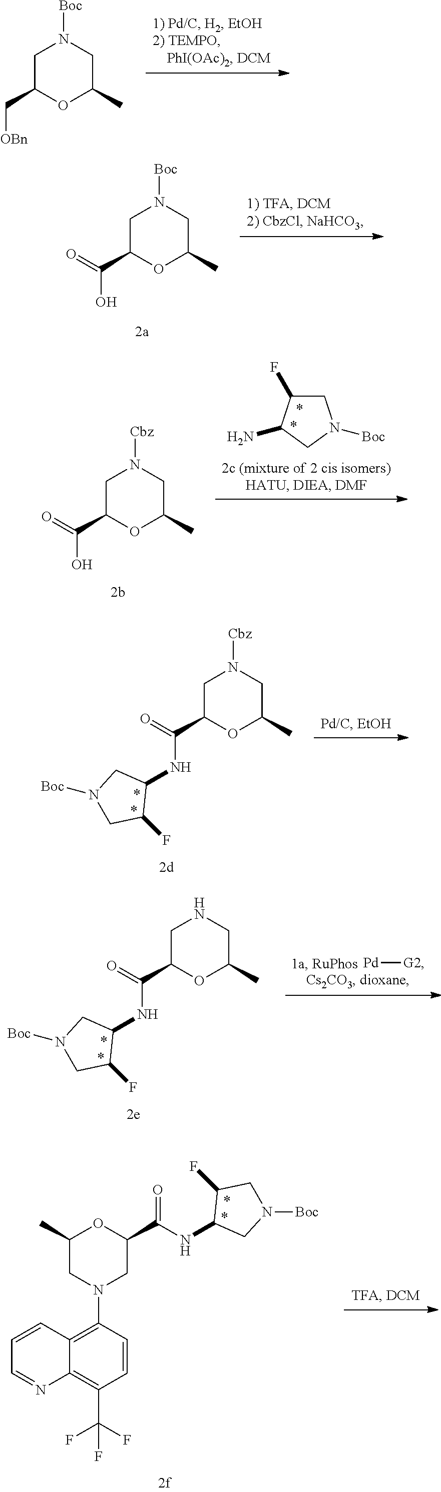

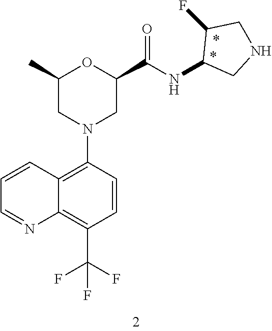

Step 1: Preparation of (2R,6R)-4-tert-butoxycarbonyl-6-methyl-morpholine-2-carboxylic acid (Compound 2a)

To a solution of tert-butyl (2R,6R)-2-(benzyloxymethyl)-6-methyl-morpholine-4-carboxylate (Reference: US 20150105370 A1) (22.0 g, 68.4 mmol) in EtOH (500 mL) was added Pd/C (7.28 g, 10% wet) and stirred for 48 h at 30° C. under H₂ atmosphere. The solution was filtered, and the filtrate was concentrated to give intermediate (15 g) as a colorless oil which was dissolved in DCM/H₂O (450 mL, v/v=4:1). To the solution was added iodobenzene diacetate (41.8 g, 130 mmol) and tetramethylpiperidinooxy (2.03 g, 13.0 mmol) at 0° C. The mixture was stirred at 0° C. for additional 0.5 h, then solvent was removed in reduced pressure and H₂O (500 mL) was added. The mixture was quenched by adding sat Na₂CO₃ to pH around 9 at 0° C., then extracted with EtOAc. The aqueous phase was acidified with citric acid to pH around 3 at 0° C., and extracted with EtOAc. The organic layer was washed with water and brine, dried over Na₂SO₄, and concentrated to give compound 2a (10 g) as a white solid. ¹NMR (400 MHz, METHANOL-d₄) δ =4.24 (d, J=12.2 Hz, 1H), 4.11 (dd, J=3.0, 11.0 Hz, 1H), 3.92 (d, J=13.4 Hz, 1H), 3.67−3.56 (m, 1H), 2.91−2.68 (m, 1H), 2.55 (m, 1H), 1.50 (s, 9H), 1.24 (d, J=6.2 Hz, 3H).

Step 2: Preparation of (2R,6R)-4-benzyloxycarbonyl-6-methyl-morpholine-2-carboxylic acid (Compound 2b)

To a solution of (2R,6R)-4-tert-butoxycarbonyl-6-methyl-morpholine-2-carboxylic acid (compound 2a, 200 mg, 0.81 mmol) in DCM (8 mL) was added TFA (6 mL) dropwise at 0° C. The reaction mixture was stirred at rt for 2 h, then concentrated and dried in reduced pressure to afford a crude product (231 mg) as a yellow gum which was dissolved in THF/H₂O (16 mL, v/v =1:1). To the solution was added NaHCO₃ (340 mg, 4.0 mmol) and then CbzCl (420 mg, 2.46 mmol) dropwise at 0° C. After addition, the mixture was stirred at rt for 12 h, then the pH was adjusted to 8-9 by adding aq. Na₂CO₃ (4 M). The aqueous solution was extracted with EtOAc (100 mL), then acidified with aq. HCl (1 N) to pH around 3, and extracted with EtOAc (100 mL) twice. The organic phase was dried and concentrated to afford crude compound 2b (230 mg). MS: calc'd 280 (MI-1¹), measured 280 (MH⁺).

Step 3: Preparation of cis-benzyl (2R,6R)-2-[(1-tert-butoxycarbonyl-4-fluoro-pyrrolidin-3-yl)carbamoyl]-6-methyl-morpholine-4- carboxylate (Compound 2d)

To a solution of (2R,6R)-4-benzyloxycarbonyl-6-methyl-morpholine-2-carboxylic acid (compound 2b, 230 mg, crude), cis-tert-butyl 3-amino-4-fluoro-pyrrolidine-1-carboxylate (CAS: 1431720-86-3, compound 2c, PharmaBlock, Cat. #: PBY2010177, 201 mg, 0.98 mmol) and DIPEA (318 mg, 2.5 mmol) in DMF (8 mL) was added HATU (436 mg, 1.15 mmol) at 0° C. The reaction mixture was stirred at rt for 2h, then diluted with H₂O (50 mL). The mixture was extracted with EtOAc and the organic layer was dried over Na₂SO₄, filtered and concentrated. The residue was purified by silica gel chromatography eluted with PE: EA=5:1-1:1 to give compound 2d (230 mg) as a colorless gum. MS: calc'd 466 (MH⁺), measured 366 (MH⁺-100).

Step 4: Preparation of cis-tert-butyl 3-fluoro-4-[[(2R,6R)-6-methylmorpholine-2-carbonyl]amino]pyrrolidine-1-carboxylate (Compound 2e)

To a solution of cis-benzyl (2R,6R)-2-[(1-tert-butoxycarbonyl-4-fluoro-pyrrolidin-3-yl)carbamoyl]-6-methyl-morpholine-4-carboxylate (compound 2d, 230 mg, 0.49 mmol) in EtOH (10 mL) was added Pd/C (30 mg, 10%, wet). The mixture was stirred at rt for 5 h under H₂ at 15 psi. The mixture was filtered. The filtrate was concentrated and dried in reduced pressure to give crude compound 2e (160 mg) as a colorless oil. MS: calc'd 332 (MH⁺), measured 276 (MH⁺-56).

Step 5: Preparation of cis-tert-butyl 3-(fluoro-4-[[(2R, 6R)-6-methyl-4-[8-(trifluoromethyl)-5-quinolyl]morpholine-2-carbonyl]amino]pyrrolidine-1-carboxylate (Compound 2f)

To a solution cis-tert-butyl 3-fluoro-4-[[(2R,6R)-6-methylmorpholine-2-carbonyl]amino]pyrrolidine-1-carboxylate (compound 2e, 40 mg, 0.12 mmol) in 1,4-dioxane (5 mL) was added 5-bromo-8-(trifluoromethyl)quinoline (compound 1a, 33 mg, 0.12 mmol), Cs₂CO₃ (78 mg, 0.24 mmol) and RuPhos Pd-G2 (CAS: 1375325-68-0, 18 mg, 0.024 mmol). The mixture was stirred at 95° C. for 5 h under N₂. The reaction mixture was concentrated, the residue was purified by prep-TLC (EA: PE=1:2) to give compound 2f (42 mg) as a yellow solid. MS: calc'd 527 (MH⁺), measured 527 (MH⁺)

Step 6: Preparation of cis-(2R,6R)-N-(4-fluoropyrrolidin-3-yl)-6-methyl-4-[8-(trifluoromethyl)-5-quinolyl]morpholine-2-carboxamide (Example 2)

To a solution of cis-tert-butyl 3-fluoro-4-[[(2R,6R)-6-methyl-4-[8-(trifluoromethyl)-5-quinolyl]morpholine-2-carbonyl]amino]pyrrolidine-1-carboxylate (compound 2f, 12 mg, 0.023 mmol) in DCM (8 mL) was added TFA (5 mL) dropwise at 0° C. The reaction mixture was stirred at rt for 2 h, and then concentrated. The residue was dissolved in water (20 mL) and the mixture was extracted with EtOAc (20 mL). The aqueous layer was dried by lyophilization to give Example 2 (4.5 mg) as a yellow solid. MS: calc'd 427 (MH⁺), measured 427 (MH⁺). ¹H NMR (400 MHz, METHANOL-d₄) δ=8.98 (dd, J=1.7, 4.3 Hz, 1H), 8.72 (dd, J=1.7, 8.6 Hz, 1H), 8.09 (d, J=8.2 Hz, 1H), 7.66 (dd, J=4.2, 8.6 Hz, 1H), 7.30 (d, J=7.9 Hz, 1H), 5.40−5.24 (m, 1H), 4.83×4.69 (m, 1H), 4.60 (dd, J=2.7, 10.6 Hz, 1H), 4.25−4.14 (m, 1H), 3.80−3.62 (m, 4H), 3.45 (t, J=11.2 Hz, 1H), 3.40−3.36 (m, 1H), 2.88−2.81(m, 1H), 2.74 (m, 1H), 1.37 (d, J=6.2 Hz, 3H).

Example 2A and 2B (separated two single isomers): (2R,6R)-N-[(3R,4S)-4-fluoropyrrolidin-3-yl]-6-methyl-4-[8-(trifluoromethyl)-5-quinolyl]morpholine-2-carboxamide and (2R,6R)-N-[(3S,4R)-4-fluoropyrrolidin-3-yl]-6-methyl-4-[8-(trifluoromethyl)-5-quinolyl]morpholine-2-carboxamide

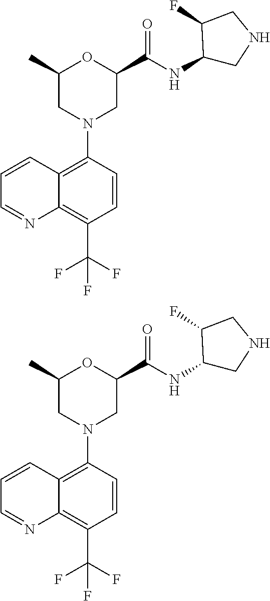 [SMILES: C[C@@H]1CN(c2ccc(C(F)(F)F)c3ncccc23)C[C@H](C(=O)N[C@@H]2CNC[C@@H]2F)O1 ; C[C@@H]1CN(c2ccc(C(F)(F)F)c3ncccc23)C[C@H](C(=O)N[C@H]2CNC[C@H]2F)O1]

Preparation of Example 2A & 2B:

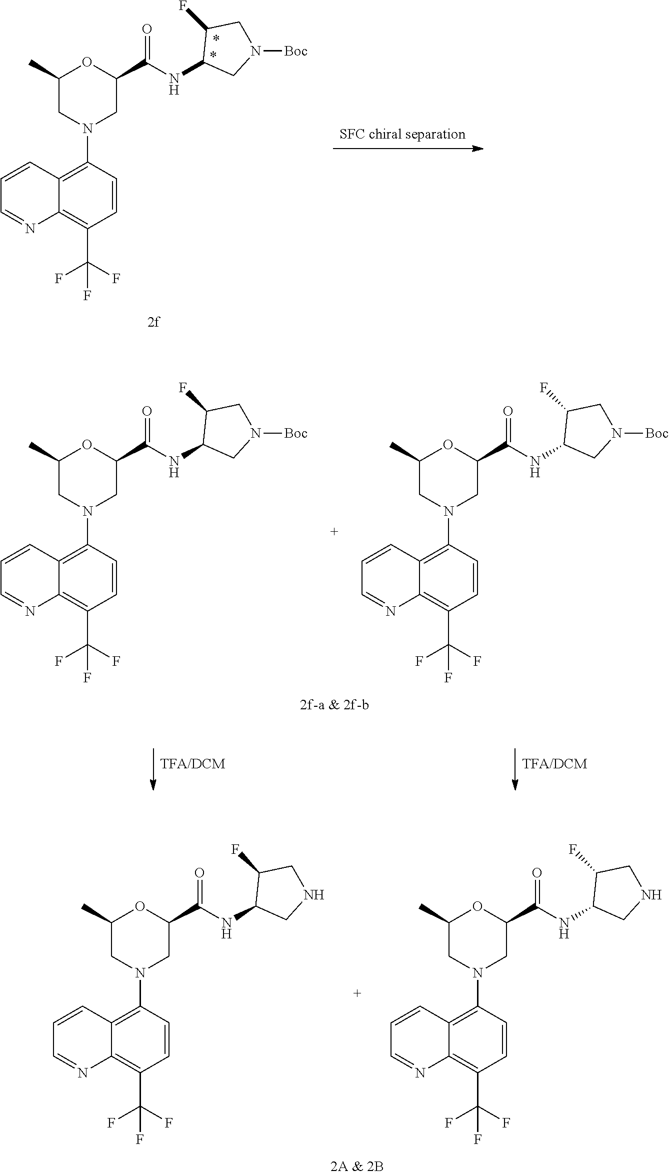

SFC chiral separation of intermediate 2f (30 mg) gave two single isomers: 2f-a (RT: 1.842 min, 13.5 mg) and 2f-b (RT: 2.244 min, 12 mg) with 30% MeOH (0.1% NH₃ H₂O)/CO₂ on AS (10 μm, 3×250 mm) column. MS: calc'd 527 (MH⁺), measured 527 (MH⁺).

To a solution of compound 2f-a (13.5 mg, 0.025 mmol) in CH₂Cl₂ (8 mL) was added TFA (5 mL) dropwise at 0° C. After addition, the mixture was stirred at rt for 2 h, then concentrated. The residue was dissolved in pure water (5 mL), and dried by lyophilization to afford Example 2A (8.1 mg) as a yellow solid. MS: calc'd 427 (MH⁺), measured 427 (MH⁺). ¹H NMR (400 MHz, METHANOL-d₄) δ=8.98 (dd, J=1.7, 4.3 Hz, 1H), 8.72 (dd, J=1.7, 8.6 Hz, 1H), 8.09 (d, J−8.2 Hz, 1H), 7.66 (dd, J=4.2, 8.6 Hz, 1H), 7.30 (d, J=7.9 Hz, 1H), 5.40−5.24 (m, 1H), 4.83−4.69 (m, 1H), 4.60 (dd, J=2.7, 10.6 Hz, 1H), 4.25−4.14 (m, 1H), 3.80−3.62 (m, 4H), 3.45 (t, J=11.2 Hz, 1H), 3.40−3.36 (m, 1H), 2.88−2.81 (m, 1H), 2.74 (m, 1H), 1.37 (d, J=6.2 Hz, 3H).

Example 2B was prepared in analogy to Example 2A as a yellow solid (6.1 mg). MS: calc'd 427 (MN⁺), measured 427 (MH⁺). 1H NMR (400 MHz, METHANOL-d₄) δ=8.98 (dd, J=1.7, 4.3 Hz, 1H), 8.72 (dd, J=1.7, 8.6 Hz, 1H), 8.09 (d, J=8.2 Hz, 1H), 7.66 (dd, J=4.2, 8.6 Hz, 1H), 7.30 (d, J=7.9 Hz, 1H), 5.40−5.24 (m, 1H), 4.83−4.69 (m, 1H), 4.60 (dd, J=2.7, 10.6 Hz, 1H), 4.25−4.14 (m, 1H), 3.80−3.62 (m, 4H), 3.45 (t, J=11.2 Hz, 1H), 3.40−.36 (m, 1H), 2.88−2.81 (m, 1H), 2.74 (m, 1H), 1.37 (d, J=6.2 Hz, 3H).

### Example 3

(2R,6R)-6-methyl-N-[(4-methylmorpholin-2-yl)methyl]-4-[8-(trifluoromethyl)-5-quinolyl]morpholine-2-carboxamide

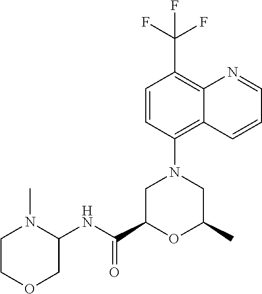 [SMILES: C[C@@H]1CN(c2ccc(C(F)(F)F)c3ncccc23)C[C@H](C(=O)NC2COCCN2C)O1]

The title compound was prepared according to the scheme below.

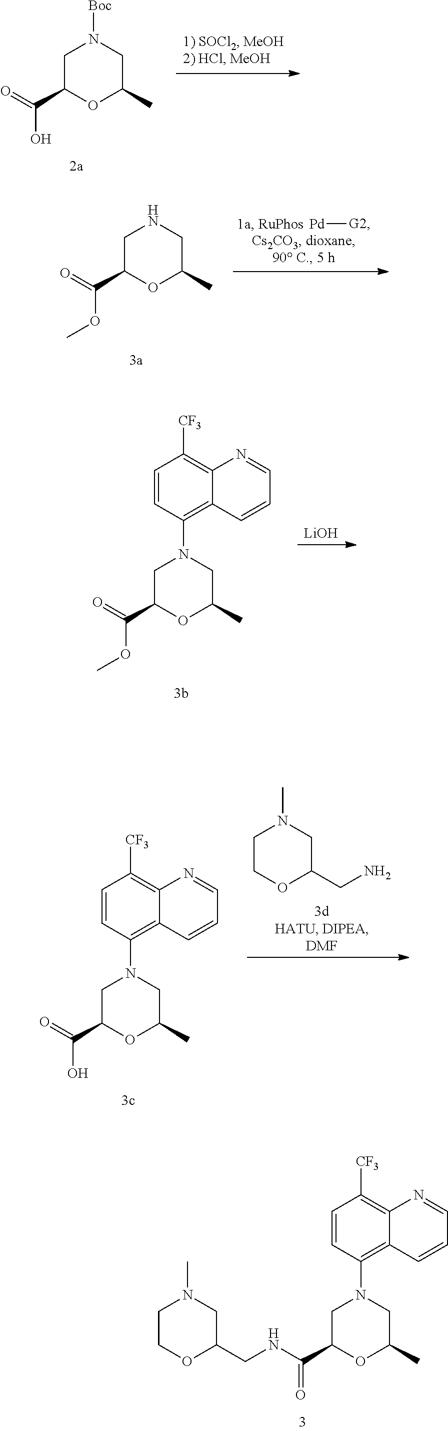

Step 1: Preparation of methyl (2R,6R)-6-methylmorpholine-2-carboxylate (compound 3a)

To a suspension of (2R,6R)-4-tert-butoxycarbonyl-6-methyl-morpholine-2-carboxylic acid (compound 2a, 1.0 g, 4.1 mmol) in MeOH (50 mL) was added SOCl₂ (1.45 g, 12.2 mmol) under ice-bath. After stirred at rt for 2 h, the reaction mixture was treated with HCl solution (4 M in MeOH, 50 mL) and stirred for additional 1 h, then concentrated to give compound 3a (780 mg) as a white solid which was directly used for next step.

Step 2: Preparation of methyl (2R,6R)-6-methyl-4-[8-(trifluoromethyl)-5-quinolyl]morpholine-2-carboxylate (Compound 3b)

To a solution of methyl (2R,6R)-6-methylmorpholine-2-carboxylate (compound 3a, 319 mg, 1.63 mmol) and 5-bromo-8-(trifluoromethyl)quinoline (compound 1a, 450 mg, 1.63 mmol) in 1,4-dioxane (15 mL) was added Cs₂CO₃ (1.59 g, 4.89 mmol). The mixture was degassed three times, then Ruphos Pd G2 (100.0 mg, 0.13 mmol) was added. The reaction mixture was stirred at 80° C. for 5 h under N₂, then cooled to rt, diluted with DCM (100 mL) and filtered. The filtrate was concentrated, and the residue was purified by silica-gel column chromatography (PE/EtOAc =10/1˜5/1) to give compound 3b (490 mg) as a yellow solid. MS: calc'd 355 (MH⁺), measured 355 (MH^{+).}

Step 3: Preparation of (2R,6R)-6-methyl-4-[8-(trifluoromethyl)-5-quinolyl]morpholine-2-carboxylic acid (Compound 3c)

To a solution of methyl (2R,6R)-6-methyl-4-[8-(trifluoromethyl)-5-quinolyl]morpholine-2-carboxylate (compound 3b, 490 mg, 1.38 mmol) in THF (5 mL) was added LiOH.H₂O (58 mg, 1.38 mmol) in water (1 mL) under ice bath. The reaction mixture was stirred at rt for 2 h, then diluted with THF (100 mL). After acidified with aq. HCl solution to pH around 6, the mixture was concentrated, and the residue was dissolved in DCM/MeOH (30 mL, v/v=20/1) and filtered. The filtrate was concentrated to give compound 3c (470 mg) as a yellow foam. MS: calc'd 341 (MW), measured 341 (MM.

Step 4: (2R,6R)-6-methyl-N-[(4-methylmorpholin-2-yl)methyl]-4-[8-(trifluoromethyl)-5-quinolyl]morpholine-2-carboxamide (Example 3)

To a solution of (2R,6R)-6-methyl-4-[8-(trifluoromethyl)-5-quinolyl]morpholine-2-carboxylic acid (compound 3c, 20.0 mg, 0.06 mmol) and (4-methylmorpholin-2-yl)methanamine (compound 3d, 7.7 mg, 0.06 mmol) in DMF (1.0 mL) was added DIPEA (0.03 mL, 0.18 mmol) and HATU (24.6 mg, 0.06 mmol). The reaction mixture was stirred at rt for 12 h then concentrated. The residue was purified by prep-HPLC to give Example 3 (9.2 mg) as a yellow solid. MS: calc'd 453 (MH⁺), measured 453 (MH⁺). ¹ H NMR (400 MHz, METHANOL-d₄) δ=9.01−8.95 (m, 1H), 8.75−8.68 (m, 1H), 8.12 (d, J=8.0 Hz, 1H), 7.69−7.62 (m, 1H), 7.31−7.25 (m, 1H), 4.59−4.51 (m, 1H), 4.23−4.12 (m, 2H), 3.90−3.76 (m, 2H), 3.72−3.64 (m, 1H), 3.55−3.39 (m, 4H), 3.42−3.33 (m, 1H), 3.23−3.10 (m, 1H), 2.94 (s, 3H), 2.92−2.70 (m, 3H), 1.36 (d, J=6.4 Hz, 3H).

### Example 4

(2R,6R)-6-methyl-N-[(1-methyl-2-piperidyl)methyl]-4-[8-(trifluoromethyl)-5-quinolyl]morpholine-2-carboxamide

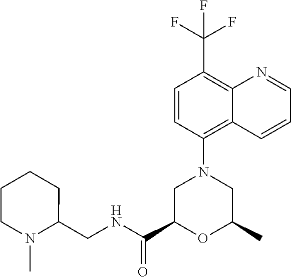 [SMILES: C[C@@H]1CN(c2ccc(C(F)(F)F)c3ncccc23)C[C@H](C(=O)NCC2CCCCN2C)O1]

The title compound was prepared in analogy to the preparation of Example 3 by using (1-methyl-2-piperidyl)methanamine instead of (4-methylmorpholin-2-yl)methanamine (compound 3d). Example 4 (8.2 mg) was obtained as a yellow solid. MS: calc'd 451 (MH⁺), measured 451 (MH⁺). ¹H NMR(400 MHz, METHANOL-d₄) δ=9.02−8.96 (m, 1H), 8.75−8.70 (m, 1H), 8.08 (d, J=8.0 Hz, 1H), 7.69−7.62 (m, 1H), 7.29 (d, J=8.0 Hz, 1H), 4.60−4.52 (m, 1H), 4.23−4.12 (m, 1H), 3.90−3.48 (m, 4H), 3.42−3.35 (m, 1H), 3.26−3.15 (m, 1H), 3.07 (s, 1.5H), 3.05 (s, 1.5H), 2.95 (d, J=9.2 Hz, 1H), 2.88−2.67 (m, 2H), 2.09−2.00 (m, 1H), 1.98−1.85 (m, 2H), 1.84−1.55 (m, 3H), 1.38 (d, J=6.4 Hz, 3H).

### Example 5 (2R,6R)-6-methyl-N-(1-methyl-4-piperidyl)-4-[8-(trifluoromethyl)-5-quinolyl]morpholine-2-carboxamide

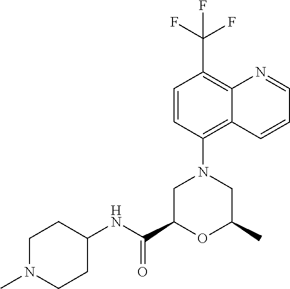 [SMILES: C[C@@H]1CN(c2ccc(C(F)(F)F)c3ncccc23)C[C@H](C(=O)NC2CCN(C)CC2)O1]

The title compound was prepared in analogy to the preparation of Example 3 by using 1-methylpiperidin-4-amine instead of (4-methylmorpholin-2-yl)methanamine (compound 3d). Example 5 (16.1 mg) was obtained as a white solid. MS: calc'd 437 (MH⁺), measured 437 (MH⁺). ¹H NMR (400 MHz, METHANOL-d₄) δ=8.98 (dd, J=1.6, 4.1 Hz, 1H), 8.71 (dd, J=1.6, 8.7 Hz, 1H), 8.08 (d, J=8.0 Hz, 1H), 7.65 (dd, J=4.3, 8.7 Hz, 1H), 7.28 (d, J=8.0 Hz, 1H), 4.51 (d, J=8.5 Hz, 1H), 4.22−4.13 (m, 1H), 4.08−3.98 (m, 1H), 3.68−3.48 (m, 3H), 3.45−3.36 (m, 1H), 3.21−3.09 (m, 2H), 2.90 (s, 3H), 2.85−2.68 (m, 2H), 2.20−2.09 (m, 2H), 1.99−1.85 (m, 2H), 1.37 (d, J=6.2 Hz, 3H).

### Example 6

(2R,6R)-N-(2-amino-2-methyl-propyl)-6-methyl-4-[8-(trifluoromethyl)-5-quinolyl]morpholine-2-carboxamide

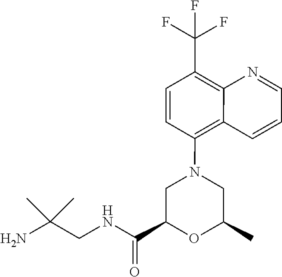 [SMILES: C[C@@H]1CN(c2ccc(C(F)(F)F)c3ncccc23)C[C@H](C(=O)NCC(C)(C)N)O1]

The title compound was prepared in analogy to the preparation of Example 3 by using 2-methylpropane-1,2-diamine instead of (4-methylmorpholin-2-yl)methanamine (compound 3d). Example 6 (10.2 mg) was obtained as a yellow solid. MS: calc'd 411 (MH⁺), measured 411 (MH⁺). ¹H NMR (400 MHz, METHANOL-d₄) δ=8.86 (dd, J=1.7, 4.2 Hz, 1H), 8.60 (dd, J=1.7, 8.6 Hz, 1H), 7.97 (d, J=7.9 Hz, 1H), 7.53 (dd, J=4.2, 8.6 Hz, 1H), 7.17 (d, J=7.9 Hz, 1H), 4.47 (dd, J=2.6, 10.6 Hz, 1H), 4.10−4.06 (m,1H), 3.58 (td, J=2.0, 11.8 Hz, 1H), 3.28 (d, J=14.4, 1H), 3.29−3.23 (m, 2H), 2.73 (t, J=11.3 Hz, 1H), 2.62 (dd, J=10.4, 11.9 Hz, 1H), 1.29−1.22 (m, 9H).

### Example 7 (3-aminoazetidin-1-yl)-[(2R,6R)-6-methyl-4-[8-(trifluoromethyl)-5-quinolyl]morpholin-2-yl]methanone

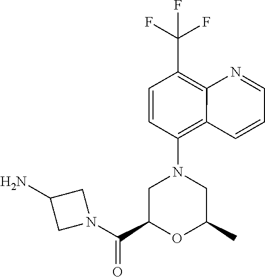 [SMILES: C[C@@H]1CN(c2ccc(C(F)(F)F)c3ncccc23)C[C@H](C(=O)N2CC(N)C2)O1]

The title compound was prepared according to the scheme below.

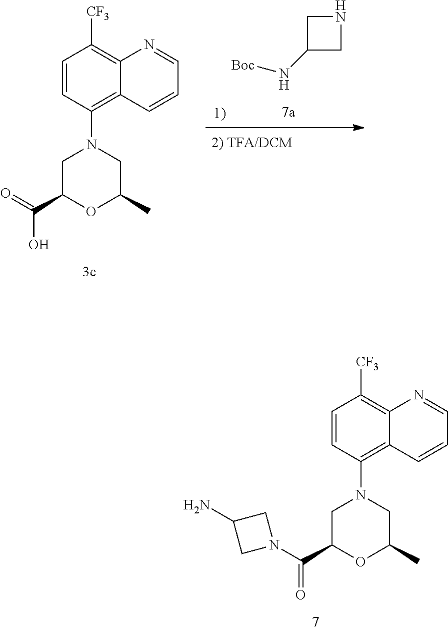

Preparation of (3-aminoazetidin-1-yl)-[(2R,6R)-6-methyl-4-[-(trifluoromethyl)-5-quinolyl]morpholin-2-yl]methanone (Example 7)

To a solution of (2R,6R)-6-methyl-4-[8-(trifluoromethyl)-5-quinolyl]morpholine-2-carboxylic acid (compound 3c, 20.0 mg, 0.06 mmol) and tert-butyl N-(azetidin-3-yl)carbamate (CAS: 91188-13-5, compound 7a, 12.3 mg, 0.06 mmol) in DMF (1.0 mL) was added DIPEA (0.03 mL, 0.18 mmol) and HATU (24.6 mg, 0.06 mmol). The mixture was stirred at rt for 12 h, then concentrated. The residue was then dissolved in DCM (2.0 mL), to which was added TFA (0.5 mL). After stirred at rt for 2 h, the reaction mixture was then concentrated to give a crude product which was purified by prep-HPLC to afford Example 7 (5.2 mg) as a yellow solid. MS:

calc'd 395 (MH⁺), measured 395 (MIT). ¹H NMR (400 MHz, METHANOL-d₄) δ=9.00−8.94 (m, 1H), 8.72−8.65 (m, 1H), 8.08 (d, J=8.0 Hz, 1H), 7.69−7.62 (m, 1H), 7.30−7.24 (m, 1H), 4.90−4.81 (m, 1H), 4.73−4.62 (m, 1H), 4.55−4.46 (m, 1H), 4.45−4.34 (m, 1H), 4.23−4.09 (m, 2H), 4.06−3.98 (m, 1H), 3.62−3.53 (m, 1H), 3.37−3.31 (m, 1H), 2.99−2.87 (m, 1H), 2.75+2.66 (m, 1H), 1.32 (d, J=6.0 Hz, 3H).

### Example 8 (2R,6R)-N-(azepan-4-yl)-6-methyl-4-[8-(trifluoromethyl)-5-quinolyl]morpholine-2-carboxamide

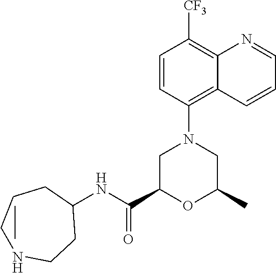

The title compound was prepared in analogy to the preparation of Example 7 by using tert-butyl 4-aminoazepane-1-carboxylate instead of tert-butyl N-(azetidin-3-yl)carbamate (compound 7a). Example 8 (15.8 mg) was obtained as a yellow solid. MS: calc'd 437 (MH⁺), measured 437 (MH⁺). ¹H NMR (400 MHz, METHANOL-d₄) δ=8.97 (dd, J=1.7, 4.2 Hz, 1H), 8.71 (dd, J=1.7, 8.6 Hz, 1H), 8.08 (d, J=7.9 Hz, 1H), 7.65 (dd, J=4.2, 8.6 Hz, 1H), 7.28 (d, J=7.9 Hz, 1H), 4.50 (dd, J=2.4, 10.6 Hz, 1H), 4.23−4.13 (m, 1H), 4.10−3.99 (m, 1H), 3.65 (d, J=11.9 Hz, 1H), 3.42−3.34 (m, 3H), 3.24−3.15 (m, 2H), 2.83−2.68 (m, 2H), 2.23−1.97 (m, 4H), 1.92−1.72 (m, 2H), 1.36 (d, J=6.2 Hz, 3H).

### Example 9

(2R,6R)-6-methyl-N-(5-methyl-5-azaspiro[2.4]heptan-7-yl)-4-[8-(trifluoromethyl)-5-quinolyl]morpholine-2-carboxamide

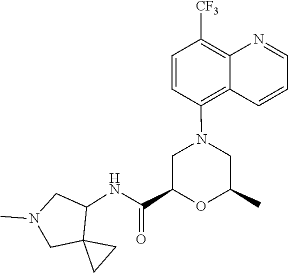 [SMILES: C[C@@H]1CN(c2ccc(C(F)(F)F)c3ncccc23)C[C@H](C(=O)NC2CN(C)CC23CC3)O1]

The title compound was prepared according to the scheme below.

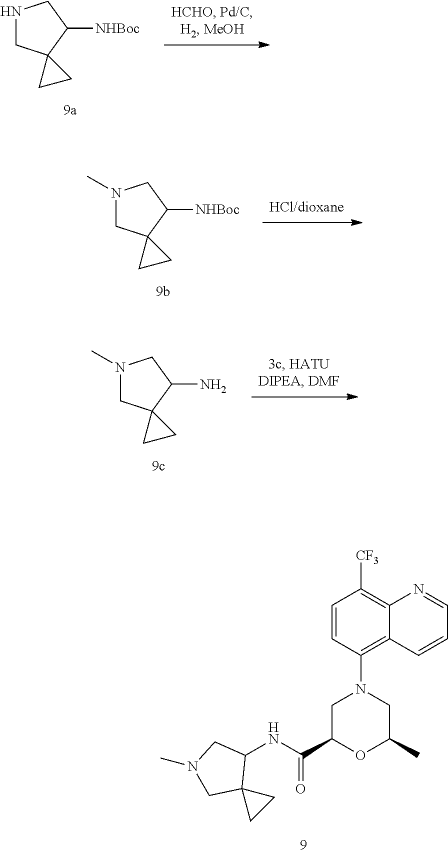

Step 1: Preparation of tert-butyl N-(5-methyl-5-azaspiro[2.41]heptan-7-yl)carbamate (Compound 9b)

To the solution of tert-butyl N-(5-azaspiro[2.4]heptan-7-yl)carbamate (CAS: 152513-88-7, compound 9a, 150 mg, 0.710 mmol) in MeOH (10 mL) was added formaldehyde (400 mg, 4.93 mmol) and Pd/C (10 mg, 10% wet). The reaction mixture was charged with 1 atm H₂ and stirred at rt for 12 h, then filtered, and the filtrate was concentrated to give crude compound 9b (151 mg) as a colorless gum. MS: calc'd 227 (MH⁺), measured 227 (MH⁺).

Step 2: Preparation of N,5-dimethyl-5-azaspiro[2.41]heptan-7-amine (Compound 9c)

To a solution of tert-butyl N-(5-methyl-5-azaspiro[2.4]heptan-7-yl)carbamate (compound 9b, 151 mg, 0.67 mmol) in DCM (10 mL) was added HCl solution (4 M in dioxane, 5 mL) dropwise at 0° C. After addition, the mixture was stirred at rt for 6h, then concentrated to give crude compound 9c (134 mg) as a yellow gum. MS: calc'd 127 (MH⁺), measured 127 (MH⁺).

Step 3: Preparation of (2R,6R)-6-methyl-N-(5-methyl-5-azaspiro[2.4]heptan-7-yl)-4-[8-(trifluoromethyl)-5-quinolyl]morpholine-2-carboxamide (Example 9)

To a solution of (2R,6R)-6-methyl-4-[8-(trifluoromethyl)-5-quinolyl]morpholine-2-carboxylic acid (compound 3c, 30.0 mg, 0.09 mmol) and N,5-dimethyl-5-azaspiro[2.4]heptan-7-amine (compound 9c, 26 mg, 0.13 mmol) in DMF (2 mL) was added DIPEA (0.08 mL, 0.44 mmol) and HATU (44 mg, 0.11 mmol). The reaction mixture was stirred at rt for 12h, then directly purified by prep-HPLC to afford Example 9 (4.4 mg) as a yellow solid. MS: calc'd 449 (MH⁺), measured 449 (MH⁺). ¹H NMR (400 MHz, METHANOL-d₄) δ=8.98 (dd, J=1.7, 4.2 Hz, 1H), 8.71 (dd, J=1.7, 8.7 Hz, 1H), 8.08 (d, J=8.1 Hz, 1H), 7.65 (dd, J=4.2, 8.6 Hz, 1H), 7.29 (d, J=8.1 Hz, 1H), 4.56 (m, 1H), 4.36−4.09 (m, 2.5H), 3.94−3.82 (m, 0.5H), 3.67 (m, 2.5H), 3.37 (m, 2.5H), 3.04 (s, 1.5H), 3.01 (s, 1.5H), 2.85−2.68 (m, 2H), 1.40−1.30 (m, 3H), 1.03−0.75 (m, 4H).

### Example 10

(3-aminopyrrolidin-1-yl)-[(2R,6R)-6-methyl-4-[8-(trifluoromethyl)-5-quinolyl]morpholin-2-yl]methanone

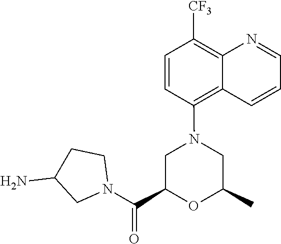 [SMILES: C[C@@H]1CN(c2ccc(C(F)(F)F)c3ncccc23)C[C@H](C(=O)N2CCC(N)C2)O1]

The title compound was prepared in analogy to the preparation of Example 7 by using tert-butyl N-pyrrolidin-3-ylcarbamate instead of tert-butyl N-(azetidin-3-yl)carbamate (CAS: 91188-13-5, compound 7a). Example 10 (16.3 mg) was obtained as a yellow solid. MS: calc'd 409 (MH⁺), measured 409 (MH⁺). ¹H NMR (400 MHz, METHANOL-d₄) δ=8.97 (d, J=8.0 Hz, 1H), 8.72−8.66 (m, 1H), 8.09 (d, J=8.0 Hz, 1H), 7.66−7.60 (m, 1H), 7.32−7.25 (m, 1H), 4.80−4.65 (m, 1H), 4.25−4.16 (m, 1H), 4.15−3.93 (m, 2H), 3.93−3.45 (m, 4H), 3.43−3.31 (m, 1H), 3.15−3.03 (m, 1H), 2.76−2.65 (m, 1H), 2.55−2.32 (m, 1H), 2.24−2.03 (m, 1H), 1.36−1.27 (m, 3H).

### Example 11

(2R,6R)-6-methyl-N-[(4-methylmorpholin-3-yl)methyl]-4-[8-(trifluoromethyl)-5-quinolyl]morpholine-2-carboxamide

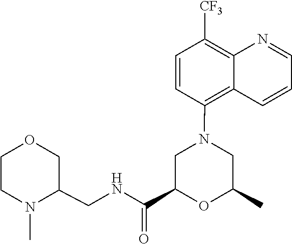 [SMILES: C[C@@H]1CN(c2ccc(C(F)(F)F)c3ncccc23)C[C@H](C(=O)NCC2COCCN2C)O1]

The title compound was prepared in analogy to the preparation of Example 3 by using (4-methylmorpholin-3-yl)methanamine instead of (4-methylmorpholin-2-yl)methanamine (compound 3d). Example 11 (9.8 mg) was obtained as a yellow solid. MS: calc'd 453 (MH⁺), measured 453 (MH⁺). ¹H NMR (400 MHz, METHANOL-d₄) 6 =8.98 (dd, J=1.8, 4.3 Hz, 1H), 8.72 (dd, J=1.7, 8.6 Hz, 1H), 8.09 (d, J=8.0 Hz, 1H), 7.66 (dd, J=4.2, 8.6 Hz, 1H), 7.30 (d, J=8.0 Hz, 1H), 4.58 (dd, J=2.6, 10.7 Hz, 1H), 4.24−3.97 (m, 3H), 3.88−3.35 (m, 9H), 3.12 (s, 3H), 2.87−2.70 (m, 2H), 1.38 (d, J=6. 3 Hz, 3H).

### Example 12

(4-amino-1-piperidyl)-[(2R,6R)-6-methyl-4-[8-(trifluoromethyl)-5-quinolyl]morpholin-2-yl]methanone

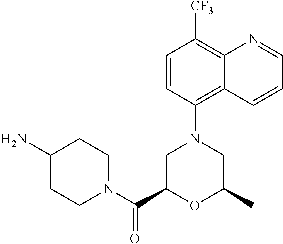 [SMILES: C[C@@H]1CN(c2ccc(C(F)(F)F)c3ncccc23)C[C@H](C(=O)N2CCC(N)CC2)O1]

The title compound was prepared in analogy to the preparation of Example 7 by using tert-butyl N-(4-piperidyl)carbamate instead of tert-butyl N-(azetidin-3-yl)carbamate (compound 7a). Example 12 (5 mg) was obtained as a brown solid. MS: calc'd 423 (MH⁺), measured 423 MH⁺). ¹H NMR (400 MHz, METHANOL-d₄) δ=8.99−8.95 (m, 1H), 8.75−8.66 (m, 1H), 8.09 (d, J=8.1 Hz, 1H), 7.62 (dd, J=4.1, 8.5 Hz, 1H), 7.31 (d, J=7.9 Hz, 1H), 4.86−4.79 (m, 1H), 4.69−4.54 (m, 1H), 4.43−4.30 (m, 1H), 4.26−4.15 (m, 1H), 3.50−3.36 (m, 3H), 3.31−3.18 (m, 1H), 3.11 (dd, J=10.4, 12.0 Hz, 1H), 2.89−2.66 (m, 2H), 2.21−2.04 (m, 2H), 1.83−1.59 (m, 1H), 1.57—1.44 (m, 1H), 1.30 (d, J=6.2 Hz, 3H).

### Example 13

(2R,6R)-6-methyl-N-(2-morpholinoethyl)-4-[8-(trifluoromethyl)-5-quinolyl]morpholine-2-carboxamide

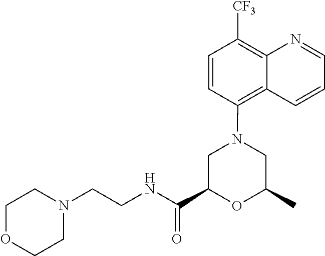 [SMILES: C[C@@H]1CN(c2ccc(C(F)(F)F)c3ncccc23)C[C@H](C(=O)NCCN2CCOCC2)O1]

The title compound was prepared in analogy to the preparation of Example 3 by using 2-morpholinoethanamine instead of (4-methylmorpholin-2-yl)methanamine (compound 3d). Example 13 (7.8 mg) was obtained as a yellow solid. MS: calc'd 453 (MH⁺), measured 453 (MH⁺). ¹H), NMR (400 MHz, METHANOL-d₄) δ=8.98 (dd, J=1.7, 4.2 Hz, 1H), 8.72 (dd, J=1.7, 8.6 Hz, 1H), 8.08 (d, J==8.1 Hz, 1H), 7.65 (dd, J=4.2, 8.6 Hz, 1H), 7.29 (d, J=7.9 Hz, 1H), 4.57 (dd, J=2.6, 10.7 Hz, 1H), 4.26−4.01 (m, 3H), 3.88−3.57 (m, 7H), 3.41−3.35 (m, 3H), 3.30−3.11 (m, 2H), 2.82 (t, J=11.3 Hz, 1H), 2.73 (dd, J=10.3, 11.9 Hz, 1H), 1.37 (d, J=6.4 Hz, 3H).

### Example 14

(2R,6R)-6-methyl-N-(1,4-oxazepan-6-yl)-4-[8-(trifluoromethyl)-5-quinolyl]morpholine-2-carboxamide

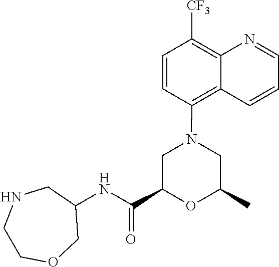 [SMILES: C[C@@H]1CN(c2ccc(C(F)(F)F)c3ncccc23)C[C@H](C(=O)NC2CNCCOC2)O1]

The title compound was prepared in analogy to the preparation of Example 7 by using tert-butyl 6-amino-1,4-oxazepane-4-carboxylate instead of tert-butyl N-(azetidin-3-yl)carbamate (compound 7a). Example 14 (9.7 mg) was obtained as a yellow solid. MS: calc'd 439 (MH⁺), measured 439 (MH⁺). ¹H NMR (400 MHz, METHANOL-d₄) δ=8.90−8.80 (m, 1H), 8.65−8.55 (m, 1H), 7.96 (d, J=7.9 Hz, 1H), 7.53 (dd, J=4.2, 8.6 Hz, 1H), 7.16 (d, J=8.1 Hz, 1H), 4.50−4.40 (m, 1H), 4.35−4.25 (m, 1H), 4.12−4.02 (m, 1H), 3.95−3.84 (m, 3H), 3.74−3.65 (m, 1H), 3.60−3.51 (m, 1H), 3.46−3.30 (m, 4H), 3.24 (d, J=12.0 Hz, 1H), 2.73−2.66 (m, 1H), 2.65−2.56 (m, 1H), 1.25 (d, J=6.2 Hz, 3H).

### Example 15

(3R,5S)-5-methyl-1-(8-methyl-5-quinolyl)piperidin-3-amine

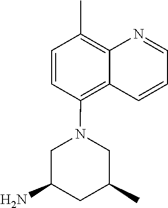 [SMILES: Cc1ccc(N2C[C@@H](C)C[C@@H](N)C2)c2cccnc12]

The title compound was prepared in analogy to the preparation of Example 1 by using 5-bromo-8-methyl-quinoline instead of 5-bromo-8-(trifluoromethyl)quinoline (compound 1a). Example 15 (5 mg) was obtained as a yellow solid. MS: calc'd 256 (MH⁺), measured 256 (MH⁺). ¹H NMR (400 MHz, METHANOL-d₄) δ=8.86 (dd, J=1.7, 4.3 Hz, 1H), 8.67−8.52 (m, 1H), 7.59−7.45 (m, 2H), 7.17 (d, J=7.7 Hz, 1H), 3.50−3.36 (m, 2H), 3.28−3.21 (m, 1H), 2.71 (s, 3H), 2.60−2.52 (m, 1H), 2.37 (t, J=11.1 Hz, 1H), 2.25−2.05 (m, 2H), 1.04 (d, J=6.5 Hz, 3H), 1.12−0.94 (m, 1H).

### Example 16

(2R,6R)-6-methyl-N-(1-methyl-4-piperidyl)-4-(8-methyl-5-quinolyl)morpholine-2-carboxamide

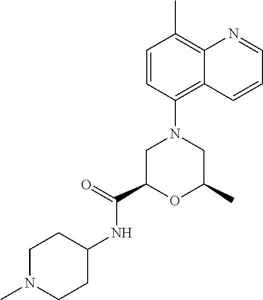 [SMILES: Cc1ccc(N2C[C@@H](C)O[C@@H](C(=O)NC3CCN(C)CC3)C2)c2cccnc12]

The title compound was prepared in analogy to the preparation of Example 3 by using 5-bromo-8-methyl-quinoline and 1-methylpiperidin-4-amine instead of 5-bromo-8-(trifluoromethyl)quinoline (compound 1a) and (4-methylmorpholin-2-yl)methanamine (compound 3d). Example 16 (7 mg) was obtained as a yellow solid. MS: calc'd 383 (MH⁺), measured 383 (MH⁺). ¹H NMR (400 MHz, METHANOL-d₄) 5 =9.50 (dd, J=1.3, 8.5 Hz, 1H), 9.14 (d, J=4.0 Hz, 1H), 8.20−8.08 (m, 1H), 8.05−7.88 (m, 1H), 7.60−7.44 (m, 1H), 4.53 (dd, J=2.6, 10.6 Hz, 1H), 4.25'14.13 (m, 1H), 4.09−3.97 (m, 1H), 3.6−23.50 (m, 3H), 3.31−3.25 (m, 1H), 3.20−3.10 (m, 2H), 2.88 (s, 3H), 2.82 (s, 3H), 2.88−2.63 (m, 2H), 2.26−2.04 (m, 2H), 2.01−1.82 (m, 2H), 1.37 (d, J=6.7 Hz, 3H).

### Example 17

(2R,6R)-6-methyl-N-[(4-methylmorpholin-2-yl)methyl]-4-(8-methyl-5-quinolyl)morpholine-2-carboxamide

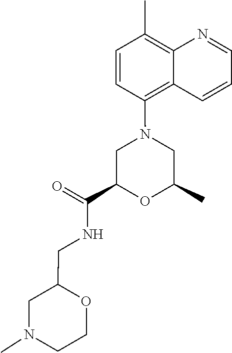 [SMILES: Cc1ccc(N2C[C@@H](C)O[C@@H](C(=O)NCC3CN(C)CCO3)C2)c2cccnc12]

The title compound was prepared in analogy to the preparation of Example 3 by using 5-bromo-8-methyl-quinoline instead of 5-bromo-8-(trifluoromethyl)quinoline (compound 1a). Example 17 (4 mg) was obtained as a yellow solid. MS: calc'd 399 (MH⁺), measured 399 (MH⁺). ¹H NMR (400 MHz, METHANOL-d₄) δ=9.49 (dd, J=1.5, 8.5 Hz, 1H), 9.14 (dd, J=1.5, 5.4 Hz, 1H), 8.18−8.07 (m, 1H), 8.03−7.92 (m, 1H), 7.54 (d, J=7.8 Hz, 1H), 4.61−4.54 (m, 1H), 4.25−4.15 (m, 2H), 3.88−3.76 (m, 2H), 3.31−3.39 (m, 5H), 3.31−3.25 (m, 2H), 2.95 (s, 3H), 2.90−2.70 (m, 3H), 2.84 (s, 3H), 1.37 (d, J=6.2 Hz, 3H).

### Example 18

(2R,6R)-6-methyl-N-[(4-methylmorpholin-2-yl)methyl]-4-(8-nitro-5-quinolyl)morpholine-2-carboxamide

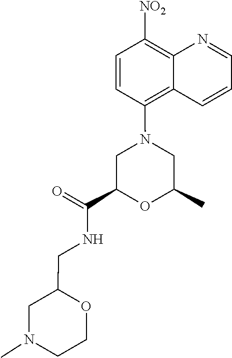 [SMILES: C[C@@H]1CN(c2ccc([N+](=O)[O-])c3ncccc23)C[C@H](C(=O)NCC2CN(C)CCO2)O1]

The title compound was prepared in analogy to the preparation of Example 3 by using 5-bromo-8-nitroquinoline instead of 5-bromo-8-(trifluoromethyl)quinoline (compound 1a). Example 18 (11 mg) was obtained as a yellow solid. MS: calc'd 430 (MH⁺), measured 430 (MH⁺). ¹H NMR (400 MHz, METHANOL-d₄) δ=8.86 (dd, J=1.7, 4.3 Hz, 1H), 8.60 (dd, J=1.7, 8.6 Hz, 1H), 8.05 (d, J=8.3 Hz, 1H), 7.57 (dd, J=4.2, 8.6 Hz, 1H), 7.16 (dd, J=1.2, 8.3 Hz, 1H), 4.41 (dd, J=2.6, 10.6 Hz, 1H), 4.10−4.01 (m, 1H), 3.84 (dd, J=3.1, 11.8 Hz, 1H), 3.65−3.47 (m, 3H), 3.35−3.22 (m, 3H), 2.86−2.58 (m, 4H), 2.30 (s, 3H), 2.22 (t, J=11.4 Hz, 1H), 2.02−1.86 (m, 1H), 1.25 (d, J=6.2 Hz, 3H).

### Example 19

cis-(2R,6R)-N-[41-fluoropyrrolidin-3-yl]-6-methyl-4-(8-nitro-5-quinolyl)morpholine-2-carboxamide (mixture of two cis diastereomers at the marked positions with *)

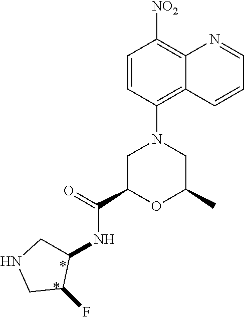 [SMILES: C[C@@H]1CN(c2ccc([N+](=O)[O-])c3ncccc23)C[C@H](C(=O)N[C@@H]2CNC[C@@H]2F)O1]

The title compound was prepared in analogy to the preparation of Example 7 by using 5-bromo-8-nitroquinoline and cis-tert-butyl 3-amino-4-fluoro-pyrrolidine-1-carboxylate (CAS:1431720-86-3, compound 2c) instead of 5-bromo-8-(trifluoromethyl)quinoline (compound la) and tert-butyl N-(azetidin-3-yl)carbamate (compound 7a). Example 19 (5 mg) was obtained as a yellow solid. MS: calc'd 404 (MH⁺), measured 404 (MM. ¹H NMR (400 MHz, METHANOL-d₄) δ=8.87 (dd, J=1.7, 4.2 Hz, 1H), 8.67−8.57 (m, 1H), 8.06 (d, J=8.2 Hz, 1H), 7.58 (dd, J=4.2, 8.6 Hz, 1H), 7.17 (dd, J=1.5, 8.4 Hz, 1H), 5.19−4.98 (m, 1H), 4.54−4.34 (m, 2H), 4.15−4.03 (m, 1H), 3.65−3.54 (m, 1H), 3.42−3.26 (m, 4H), 3.02−2.91 (m, 1H), 2.81−2.61 (m, 2H), 1.25 (d, J=6.2 Hz, 3H).

### Example 20

(2R,6R)-6-methyl-N-(1-methyl-4-piperidyl)-4-(8-nitro-5-quinolyl)morpholine-2-carboxamide

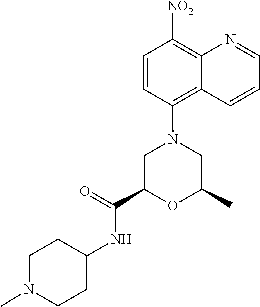 [SMILES: C[C@@H]1CN(c2ccc([N+](=O)[O-])c3ncccc23)C[C@H](C(=O)NC2CCN(C)CC2)O1]

The title compound was prepared in analogy to the preparation of Example 3 by using 5-bromo-8-nitroquinoline (compound 15a) and 1-methylpiperidin-4-amine instead of 5-bromo-8-(trifluoromethyl)quinoline (compound la) and (4-methylmorpholin-2-yl)methanamine (compound 3d). Example 20 (6 mg) was obtained as a yellow solid. MS: calc'd 414 (MH⁺), measured 414 (MH⁺). ¹H NMR (400 MHz, METHANOL-d₄) δ=8.98 (dd, J=1.7, 4.2 Hz, 1H), 8.72 (dd, J=1.7, 8.6 Hz, 1H), 8.17 (d, J=8.3 Hz, 1H), 7.69 (dd, J=4.2, 8.6 Hz, 1H), 7.28 (d, J=8.3 Hz, 1H), 4.49 (dd, J=2.6, 10.6 Hz, 1H), 4.23−4.12 (m, 1H), 3.86−3.75 (m, 1H), 3.69 (td, J=2.2, 11.9 Hz, 1H), 3.39 (td, J=2.0, 11.9 Hz, 1H), 2.96 (d, J=11.2 Hz, 2H), 2.87−2.70 (m, 2H), 2.37 (s, 3H), 2.27 (t, J=11.9 Hz, 2H), 1.91 (t, J=13.1 Hz, 2H), 1.76−1.61 (m, 2H), 1.37 (d, J=6.4 Hz, 3H).

### Example 21

cis-(2R,6R)-N-14-fluoropyrrolidin-3-yl1-6-methyl-4-(8-methyl-5-quinolyl)morpholine-2carboxamide (mixture of two cis diastereomers at the marked positions with *)

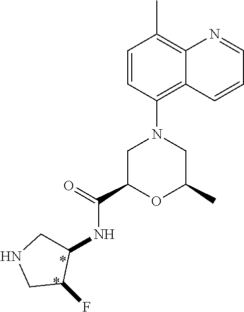 [SMILES: Cc1ccc(N2C[C@@H](C)O[C@@H](C(=O)N[C@@H]3CNC[C@@H]3F)C2)c2cccnc12]

The title compound was prepared in analogy to the preparation of Example 7 by using 5-bromo-8-methyl-quinoline and cis-tert-butyl 3-amino-4-fluoro-pyrrolidine-1-carboxylate (CAS:1431720-86-3, compound 2c) instead of 5-bromo-8-(trifluoromethyl)quinoline (compound 1a) and tert-butyl N-(azetidin-3-yl)carbamate (compound 7a) Example 21 (3 mg) was obtained as a yellow solid. MS: calc'd 373 (MH⁺), measured 373 (MH⁺). ¹H NMR (400 MHz, METHANOL-d₄) δ=8.76 (dd, J=1.7, 4.3 Hz, 1H), 8.60 (dd, J=1.7, 8.4 Hz, 1H), 7.52−7.37 (m, 2H), 7.07 (d, J=7.7 Hz, 1H), 5.15−4.93 (m, 1H), 4.47−4.27 (m, 2H), 4.14−3.95 (m, 1H), 3.45−3.15 (m, 4H), 3.15−3.03 (m, 1H), 2.93−2.78 (m, 1H), 2.69−2.47 (m, 2H), 2.60 (s, 3H), 1.22 (d, J=6.2 Hz, 3H).

### Example 22 (2R,6R)-4-(8-chloro-5-quinolyl)-N-[(3S,4R)-4-fluoropyrrolidin-3-yl]-6-methyl-morpholine-2-carboxamide

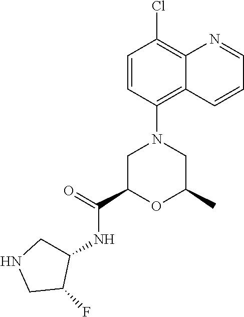 [SMILES: C[C@@H]1CN(c2ccc(Cl)c3ncccc23)C[C@H](C(=O)N[C@H]2CNC[C@H]2F)O1]

The title compound was prepared in analogy to the preparation of Example 7 by using 5-bromo-8-chloro-quinoline and tert-butyl (3S,4R)-3-amino-4-fluoro-pyrrolidine-1-carboxylate (CAS: 1174020-30-4, PharmaBlock, Cat. #: PB07374) instead of 5-bromo-8-(trifluoromethyl)quinoline (compound 1a) and tert-butyl N-(azetidin-3-yl)carbamate (compound 7a). Example 22 (12 mg) was obtained as a yellow solid. MS: calc'd 393 (MH⁺), measured 393

(MTV). ¹H NMR (400 MHz, METHANOL-d₄) δ=9.06 (dd, J=1.6, 4.6 Hz, 1H), 9.01 (dd, J=1.6, 8.6 Hz, 1H), 7.97 (d, J=8.2 Hz, 1H), 7.85 (dd, J=4.6, 8.6 Hz, 1H), 7.35 (d, J=8.2 Hz, 1H), 5.46−5.19 (m, 1H), 4.84−4.68 (m, 1H), 4.60 (dd, J=2.6, 10.7 Hz, 1H), 4.26−4.11 (m, 1H), 3.83−3.60 (m, 3H), 3.57 (td, J=2.1, 11.7 Hz, 1H), 3.45 (t, J=11.2 Hz, 1H), 3.31−3.24 (m, 1H), 2.93−2.61 (m, 2H), 1.36 (d, J=6.4 Hz, 3H).

### Example 23

(3R,5S)-1-(8-chloro-5-quinolyl)-5-methyl-piperidin-3-amine

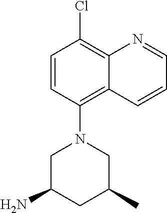 [SMILES: C[C@H]1C[C@@H](N)CN(c2ccc(Cl)c3ncccc23)C1]

The title compound was prepared in analogy to the preparation of Example 1 by using 5-bromo-8-chloro-quinoline instead of 5-bromo-8-(trifluoromethyl)quinoline (compound 1a). Example 23 (6 mg) was obtained as a yellow solid. MS: calc'd 276 (MH⁺), measured 276 (MH⁺). ¹H NMR (400 MHz, METHANOL-d₄) δ=9.00 (dd, J=1.7, 4.5 Hz, 1H), 8.77 (dd, J=1.7, 8.6 Hz, 1H), 7.91 (d, J=8.2 Hz, 1H), 7.74 (dd, J=4.5, 8.6 Hz, 1H), 7.32 (d, J=8.2 Hz, 1H), 3.75−3.64 (m, 1H), 3.63−3.54 (m, 1H), 3.51−3.30 (m, 1H), 2.78 (t, J=10.8 Hz, 1H), 2.48 (t, J=11.3 Hz, 1H), 2.35−2.14 (m, 2H), 1.25 (q, J=11.9 Hz, 1H), 1.08 (d, J=6.6 Hz, 3H).

### Example 24

(2R,6R)-4-(8-chloro-5-quinolyI)-6-methyl-N-(1-methyl-4-piperidyl)morpholine-2-carboxamide

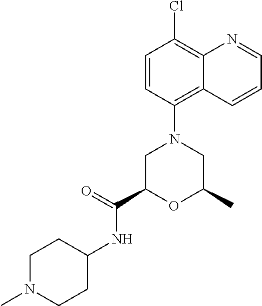 [SMILES: C[C@@H]1CN(c2ccc(Cl)c3ncccc23)C[C@H](C(=O)NC2CCN(C)CC2)O1]

The title compound was prepared in analogy to the preparation of Example 3 by using 5-bromo-8-chloro-quinoline and 1-methylpiperidin-4-amine instead of 5-bromo-8-(trifluoromethyl)quinoline (compound 1a) and (4-methylmorpholin-2-yl)methanamine (compound 3d). Example 24 (23 mg) was obtained as a yellow solid. MS: calc'd 403 (MH⁺), measured 403 (MH⁺). ¹H NMR (400 MHz, METHANOL-d₄) δ=9.05 (dd, J=1.6, 4.6 Hz, 1H), 9.00 (dd, J=1.5, 8.6 Hz, 1H), 7.97 (d, J=8.3 Hz, 1H), 7.84 (dd, J=4.6, 8.6 Hz, 1H), 7.35 (d, J=8.3 Hz, 1H), 4.50 (dd, J=2.6, 10.6 Hz, 1H), 4.16 (ddd, J=2.3, 6.3, 10.1 Hz, 1H), 4.09−3.96 (m, 1H), 3.65−3.52 (m, 3H), 3.30−3.23 (m, 1H), 3.20−3.06 (m, 2H), 2.89 (s, 3H), 2.84−2.65 (m, 2H), 2.20−2.07 (m, 2H), 2.00−1.84 (m, 2H), 1.36 (d, J=6.4 Hz, 3H).

### Example 25

(2R,6R)-N-(azepan-4-yl)-4-(8-chloro-5-quinolyl)-6-methyl-morpholine-2-carboxamide

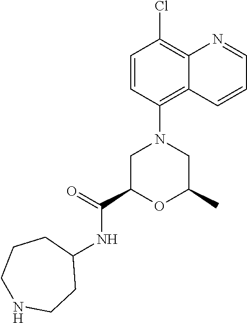 [SMILES: C[C@@H]1CN(c2ccc(Cl)c3ncccc23)C[C@H](C(=O)NC2CCCNCC2)O1]

The title compound was prepared in analogy to the preparation of Example 7 by using 5-bromo-8-chloro-quinoline and tert-butyl 4-aminoazepane-1-carboxylate instead of 5-bromo-8-(trifluoromethyl)quinoline (compound 1a) and tert-butyl N-(azetidin-3-yl)carbamate (compound 7a). Example 25 (10 mg) was obtained as a yellow solid. MS: calc'd 403 (MH⁺), measured 403 (MH⁺). ¹H NMR (400 MHz, METHANOL-d₄) δ=8.98 (dd, J=1.6, 4.3 Hz, 1H), 8.81 (dd, J=1.6, 8.6 Hz, 1H), 7.87 (d, J=8.2 Hz, 1H), 7.70 (dd, J=4.3, 8.6 Hz, 1H), 7.27 (dd, J=1.7, 8.2 Hz, 1H), 4.48 (dd, J=2.6, 10.6 Hz, 1H), 4.23−4.11 (m, 1H), 4.09−3.97 (m, 1H), 3.59−3.50 (m, 1H), 3.45−3.31 (m, 2H), 3.29−3.15 (m, 3H), 2.81−2.64 (m, 2H), 2.25−1.68 (m, 6H), 1.35 (d, J=6.2 Hz, 3H).

### Example 26

(2R,6R)-4-(8-chloro-5-quinolyl)-6-methyl-N-[(4-methylmorpholin-2-yl)methyl]morpholine-2-carboxamide

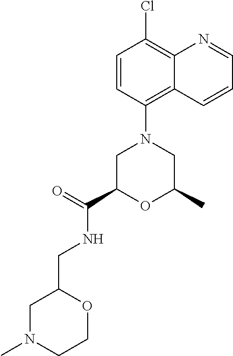 [SMILES: C[C@@H]1CN(c2ccc(Cl)c3ncccc23)C[C@H](C(=O)NCC2CN(C)CCO2)O1]

The title compound was prepared in analogy to the preparation of Example 3 by using 5-bromo-8-chloro-quinoline instead of 5-bromo-8-(trifluoromethyl)quinoline (compound 1a). Example 26 (12 mg) was obtained as a yellow solid. MS: calc'd 419 (MH⁺), measured 419 (MH⁺). ¹H NMR (400 MHz, DMSO-d₄) δ=9.02 (dd, J=1.6, 4.2 Hz, 1H), 8.62 (d, J=8.6 Hz, 1H), 7.86 (d, J=8.2 Hz, 1H), 7.87−7.80 (brs, 1H), 7.67 (dd, J=4.2, 8.4 Hz, 1H), 7.21 (d, J=8.2 Hz, 1H), 4.41 (dd, J=2.4, 10.6 Hz, 1H), 4.10−3.98 (m, 1H), 3.94−3.79 (m, 1H), 3.66−3.46 (m, 2H), 3.40−3.35 (m, 1H), 3.27−3.09 (m, 4H), 2.99−2.74 (m, 2H), 2.72−2.55 (m, 3H), 2.39 (s, 3H), 1.25 (d, J=6.2 Hz, 3H).

### Example 27

(2R,6R)-N-[[(2S,4R)-4-fluoropyrrolidin-2-yl]methyl]-6-methyl-4-[8-(trifluoromethyl)-5-quinolyl]morpholine-2-carboxamide

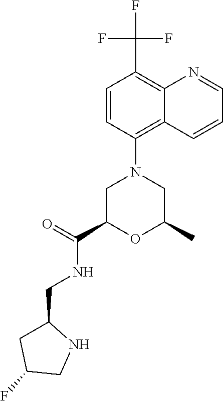 [SMILES: C[C@@H]1CN(c2ccc(C(F)(F)F)c3ncccc23)C[C@H](C(=O)NC[C@@H]2C[C@@H](F)CN2)O1]

The title compound was prepared in analogy to the preparation of Example 7 by using tert-butyl (2S,4R)-2-(aminomethyl)-4-fluoro-pyrrolidine-1-carboxylate instead of tert-butyl N-(azetidin-3-yl)carbamate (compound 7a). Example 27 (4.7 mg) was obtained as a yellow solid. MS: calc'd 441 (MH⁺), measured 441 (MH⁺). ¹H NMR (400 MHz, METHANOL-d₄) δ=8.98 (dd, J=1.5, 4.3 Hz, 1H), 8.72 (dd, J=1.6, 8.6 Hz, 1H), 8.08 (d, J=7.9 Hz, 1H), 7.65 (dd, J=4.2, 8.6 Hz, 1H), 7.29 (d, J=8.0 Hz, 1H), 5.53−5.36 (m, 1H), 4.60 (dd, J=2.6, 10.7 Hz, 1H), 4.25−4.16 (m, 1H), 4.09−3.99 (m, 1H), 3.73−3.58 (m, 5H), 3.38 (d, J=11.4 Hz, 1H), 2.83 (t, J=11.4

Hz, 1H), 2.73 (dd, J=10.5, 11.6 Hz, 1H), 2.54−2.44 (m, 1H), 2.16−1.97 (m, 1H), 1.38 (d, J=6.3 Hz, 3H).

### Example 28

(2R,6R)-N-(5,5-difluoro-3-piperidyl)-6-methyl-4-[8-(trifluoromethyl)-5-quinolyl]morpholine-2-carboxamide

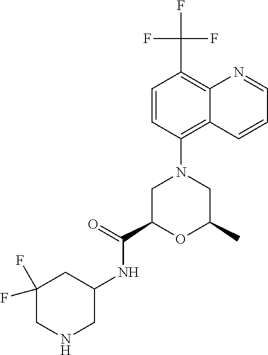 [SMILES: C[C@@H]1CN(c2ccc(C(F)(F)F)c3ncccc23)C[C@H](C(=O)NC2CNCC(F)(F)C2)O1]

The title compound was prepared in analogy to the preparation of Example 7 by using tert-butyl 5-amino-3,3-difluoro-piperidine-1-carboxylate instead of tert-butyl N-(azetidin-3-yl)carbamate (compound 7a). Example 28 (4.7 mg) was obtained as a yellow solid. MS: calc'd 459 (MH⁺), measured 459 (MH⁺). ¹H NMR (400 MHz, METHANOL-d₄) δ−8.98 (dd, J=1.6, 4.2 Hz, 1H), 8.72 (d, J=8.4, 1H), 8.08 (d, J=8.0 Hz, 1H), 7.65 (dd, J=4.2, 8.5 Hz, 1H), 7.29 (dd, J=3.5, 8.0 Hz, 1H), 4.56 (td, J=2.5, 10.7 Hz, 1H), 4.50−4.41 (m, 1H), 4.23−4.14 (m, 1H), 3.76−3.64 (m, 2H), 3.57−3.44 (m, 2H), 3.38 (m, 1H), 3.19−3.09 (m, 1H), 2.82 (dt, J=4.5, 11.3 Hz, 1H), 2.76−2.69 (m, 1H), 2.56 (m, 1H), 2.45−2.29 (m, 1H), 1.37 (d, J=6.3 Hz, 3H).

### Example 29

(2R,6R)-N-[(4,4-difluoropyrrolidin-3-yl)methyl]-6-methyl-4-[8-(trifluoromethyl)-5-quinolyl]morpholine-2-carboxamide

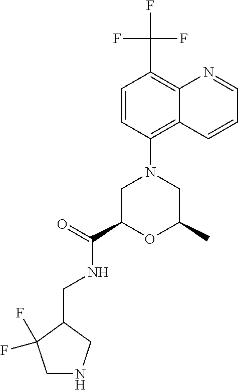 [SMILES: C[C@@H]1CN(c2ccc(C(F)(F)F)c3ncccc23)C[C@H](C(=O)NCC2CNCC2(F)F)O1]

The title compound was prepared in analogy to the preparation of Example 7 by using tert-butyl 2-(aminomethyl)-3,3-difluoro-pyrrolidine-1-carboxylate (CAS: 1627595-60-1, PharmaBlock, Cat. #: PBLJ1874) instead of tert-butyl N-(azetidin-3-yl)carbamate (compound 7a) Example 29 (9.5 mg) was obtained as a yellow solid. MS: calc'd 459 (MH⁺), measured 459

(Ann. ¹14 NMR (400 MHz, METHANOL-d₄) d^{—} 8.98 (dd, J=1.6, 4.1 Hz, 1H), 8.71 (d, J=8.7 Hz, 1H), 8.08 (d, J=8.0 Hz, 1H), 7.65 (dd, J=4.2, 8.6 Hz, 1H), 7.28 (d, J=8.0 Hz, 1H), 4.54 (d, J=9.5 Hz, 1H), 4.24−4.12 (m, 1H), 3.84−3.72 (m, 3H), 3.67 (dd, J=2.1, 11.7 Hz, 1H), 3.57 (dd, J=1.8, 6.8 Hz, 2H), 3.41−3.35 (m, 2H), 3.14−2.99 (m, 1H), 2.81−2.69 (m, 2H), 1.36 (d, J=6.3 Hz, 3H).

### Example 30

(2R,6R)-N-[[(2S)-4,4-difluoropyrrolidin-2-yl]methyl]-6-methyl-4-[-(trifluoromethyl)-5-quinolyl]morpholine-2-carboxamide

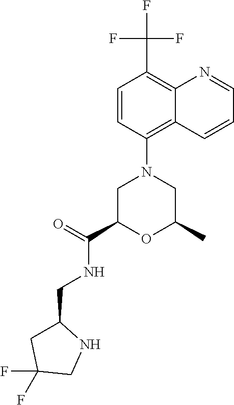 [SMILES: C[C@@H]1CN(c2ccc(C(F)(F)F)c3ncccc23)C[C@H](C(=O)NC[C@@H]2CC(F)(F)CN2)O1]

The title compound was prepared in analogy to the preparation of Example 7 by using tert-butyl (2S)-2-(aminomethyl)-4,4-difluoro-pyrrolidine-1-carboxylate (CAS: 1363384-67-1) instead of tert-butyl N-(azetidin-3-yl)carbamate (compound 7a). Example 30 (9.5 mg) was obtained as a yellow solid. MS: calc'd 459 (MH⁺), measured 459 (MH⁺). ¹H NMR (400 MHz, METHANOL-d₄) (δ8.98 (dd, J=1.7, 4.2 Hz, 1H), 8.72 (dd, J=1.7, 8.7 Hz, 1H), 8.08 (d, J=8.1 Hz, 1H), 7.65 (dd, J=4.2, 8.6 Hz, 1H), 7.29 (d, J=8.1 Hz, 1H), 4.60 (dd, J=2.6, 10.8 Hz, 1H), 4.22−4.17 (m, 1H), 4.14−4.05 (m, 1H), 3.92−3.82 (m, 1H), 3.79−3.66 (m, 4H), 3.68−3.39 (m, 1H), 2.88−2.80 (t, J=11.2 Hz, 1H), 2.79−2.69 (m, 2H), 2.54−2.40 (m, 1H), 1.38 (d, J=6.2 Hz, 3H).

### Example 31

(2R,6R)-N-(3-azabicyclo[3.2.1]octan-8-yl)-6-methyl-4-[8-(trifluoromethyl)-5-quinolyl]morpholine-2-carboxamide

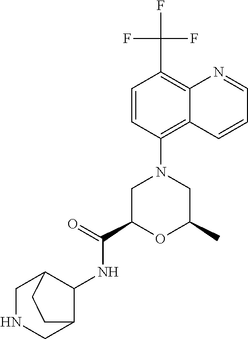 [SMILES: C[C@@H]1CN(c2ccc(C(F)(F)F)c3ncccc23)C[C@H](C(=O)NC2C3CCC2CNC3)O1]

The title compound was prepared in analogy to the preparation of Example 7 by using tert-butyl 8-amino-3-azabicyclo[3.2.1]octane-3-carboxylate (CAS: 1330763-51-3, PharmaBlock, Cat. #: PBN20120304) instead of tert-butyl N-(azetidin-3-yl)carbamate (CAS: 91188-13-5, compound 7a). Example 31 (5.2 mg) was obtained as a yellow solid. MS: calc'd 449 (MH⁺), measured 449 (MH⁺). ¹H NMR (400 MHz, METHANOL-d₄) δ=8.98 (dd, J=1.8, 4.3 Hz, 1H), 8.73 (dd, J=1.7, 8.6 Hz, 1H), 8.08 (d, J=8.2 Hz, 1H), 7.66 (dd, J=4.2, 8.6 Hz, 1H), 7.30 (d, J=8.0 Hz, 1H), 4.59 (dd, J=2.6, 10.7 Hz, 1H), 4.25−4.21 (m, 1H), 3.88 (t, J=4.4 Hz, 1H), 3.69 (td, J=2.1, 11.7 Hz, 1H), 3.39 (td, J=2.0, 12.0 Hz, 1H), 2.99 (dd, J=7.5, 13.7 Hz, 2H), 2.85 (t, J=11.6 Hz, 1H), 2.75 (dd, J=10.3, 11.9 Hz, 1H), 2.58 (td, J=3.7, 13.7 Hz, 2H), 2.25−2.15 (m, 2H), 1.95−1.85 (m, 2H), 1.84−1.77 (m, 2H), 1.38 (d, J=6.3 Hz, 3H).

### Example 32

3,9-diazaspiro[5.5]undecan-3-yl-[(2R,6R)-6-methyl-4-[8-(trifluoromethyl)-5-quinolyl]morpholin-2-yl]methanone

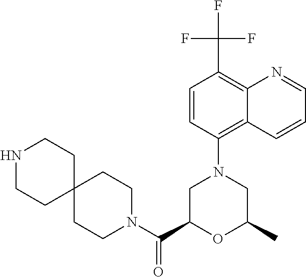 [SMILES: C[C@@H]1CN(c2ccc(C(F)(F)F)c3ncccc23)C[C@H](C(=O)N2CCC3(CCNCC3)CC2)O1]

The title compound was prepared in analogy to the preparation of Example 7 by using tert-butyl 3,9-diazaspiro[5.5]undecane-3-carboxylate instead of tert-butyl N-(azetidin-3-yl)carbamate (compound 7a). Example 32 (17.4 mg) was obtained as a yellow solid. MS: calc'd 477 (MH⁺), measured 477 (MH⁺). ¹H NMR (400 MHz, METHANOL-d₄) δ=9.08 (dd, J=1.4, 4.6 Hz, 1H), 9.04 (dd, J=1.4, 8.6 Hz, 1H), 8.25 (d, J=8.2 Hz, 1H), 7.86 (dd, J=4.7, 8.6 Hz, 1H), 7.44 (d, J=8.2 Hz, 1H), 4.85−4.80 (m, 1H), 4.22−4.18 (m, 1H), 3.85−3.72 (m, 2H), 3.69−3.61 (m, 1H), 3.55−3.46 (m, 2H), 3.42 (d, J=11.9 Hz, 1H), 3.24 (t, J=5.5 Hz, 4H), 3.17 (dd, J=10.3, 12.2 Hz, 1H), 2.77 (dd, J=10.3, 11.8 Hz, 1H), 1.82 (td, J=5.9, 17.6 Hz, 4H), 1.75−1.69 (m, 2H), 1.67−1.52 (m, 2H), 1.31 (d, J=6.1 Hz, 3H).

### Example 33

(2R,6R)-6-methyl-N-(3-oxa-9-azabicyclo[3.3.1]nonan-7-yl)-4-[8-(trifluoromethyl)-5-quinolyl]morpholine-2-carboxamide

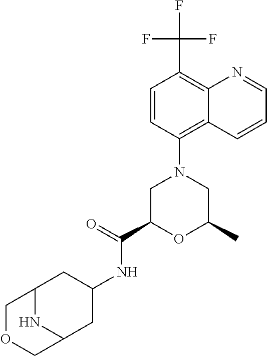 [SMILES: C[C@@H]1CN(c2ccc(C(F)(F)F)c3ncccc23)C[C@H](C(=O)NC2CC3COCC(C2)N3)O1]

The title compound was prepared in analogy to the preparation of Example 7 by using tert-butyl 7-amino-3-oxa-9-azabicyclo[3.3.1]nonane-9-carboxylate (CAS: 280762-03-0, PharmaBlock, Cat. #: PBN20120428) instead of tert-butyl N-(azetidin-3-yl)carbamate (CAS: 91188-13-5, compound 7a). Example 33 (14.6 mg) was obtained as a yellow solid. MS: calc'd 465 (MH⁺), measured 465 (MH⁺). ¹H NMR (400 MHz, METHANOL-d₄) δ9.07 (dd, J=1.6, 4.6 Hz, 1H), 8.97 (dd, J=1.4, 8.6 Hz, 1H), 8.20 (d, J=8.2 Hz, 1H), 7.84 (dd, J=4.6, 8.5 Hz, 1H), 7.39 (d, J=8.2 Hz, 1H), 4.60−4.50 (m, 2H), 4.19 (m, 1H), 4.14−4.05 (m, 4H), 3.73 (td, J=2.0, 12.0 Hz, 1H), 3.69−3.60 (m, 2H), 3.40 (td, J=2.0, 12.0 Hz, 1H), 2.83 (t, J=11.4 Hz, 1H), 2.74 (dd, J=10.3, 11.9 Hz, 1H), 2.63 −2.52 (m, 2H), 2.07−1.94 (m, 2H), 1.32 (d, J=6.3 Hz, 3H).

### Example 34

(2R,6R)-N-(9-azabicyclo[3.3.1]nonan-3-yl)-6-methyl-4-[8-(trifluoromethyl)-5-quinolyl]morpholine-2-carboxamide

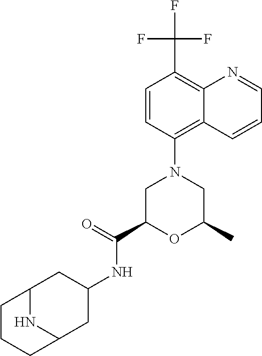 [SMILES: C[C@@H]1CN(c2ccc(C(F)(F)F)c3ncccc23)C[C@H](C(=O)NC2CC3CCCC(C2)N3)O1]

The title compound was prepared in analogy to the preparation of Example 7 by using tert-butyl 3-amino-9-azabicyclo[3.3.1]nonane-9-carboxylate (CAS: 1187927-41-8, J&W Pharmlab, Cat. #: 60R0380S) instead of tert-butyl N-(azetidin-3-yl)carbamate (compound 7a). Prep-HPLC separation gave two single isomers Example 34A (RT: 0.901 min, 5.9 mg) and Example 34B (RT: 0.923 min, 5.3 mg) as yellow solids with 23%˜43% ACN in H₂O (0.05% HCl) as eluent on Phenomenex Synergi C18 (10 μm, 25×150 mm) column.

Example 34A: MS: calc'd 463 (MET), measured 463 (MH⁺). ¹H NMR (400 MHz, METHANOL-d₄) δ=9.03 (dd, J=1.6, 4.5 Hz, 1H), 8.87 (dd, J=1.7, 8.6 Hz, 1H), 8.16 (d, J=8.2 Hz, 1H), 7.76 (dd, J=4.5, 8.6 Hz, 1H), 7.35 (d, J=8.0 Hz, 1H), 4.83−4.77 (m, 1H), 4.54 (dd, J=2.6, 10.7 Hz, 1H), 4.21−4.16 (m, 1H), 3.85−3.75 (m, 2H), 3.68 (td, J=2.0, 12.0 Hz, 1H), 3.38 (td, J=2.0, 12.0 Hz, 1H), 2.83 (dd, J=10.9, 11.7 Hz, 1H), 2.76 (dd, J=10.3, 11.9 Hz, 1H), 2.29−2.19 m, 2H), 2.18−2.00 (m, 7H), 1.85−1.78 (m, 1H), 1.37 (d, J=6.3 Hz, 3H).

Example 34B: MS: calc'd 463 (MH⁺), measured 463 (MH⁺). ¹H NMR (400 MHz, METHANOL-d₄) δ=9.09 (dd, J=1.4, 4.7 Hz, 1H), 9.04 (dd, J=1.4, 8.6 Hz, 1H), 8.24 (d, J=8.2 Hz, 1H), 7.89 (dd, J=4.7, 8.6 Hz, 1H), 7.42 (d, J=8.2 Hz, 1H), 4.54 (dd, J=2.5, 10.7 Hz, 1H), 4.34−4.25 (m, 1H), 4.24−4.16 (m, 1H), 3.90−3.80 (m, 2H), 3.71 (d, J=11.9 Hz, 1H), 3.42 (d, J=11.9 Hz, 1H), 2.86 (t, J=11.3 Hz, 1H), 2.79 (dd, J=10.5, 11.7 Hz, 1H), 2.43×2.33 (m, 2H), 2.28−2.14 (m, 1H), 1.98−1.86 (m, 2H), 1.85−1.64 (m, 5H), 1.39 (d, J=6.3 Hz, 3H).

### Example 35

(2R,6R)-6-methyl-N-[1-(4-methylmorpholin-2-yl)ethyl]-4-[8-(trifluoromethyl)-5-quinolyl]morpholine-2-carboxamide

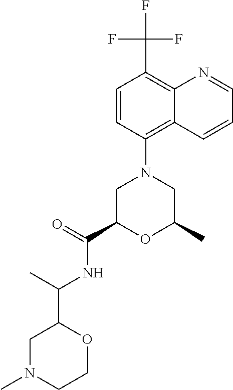 [SMILES: CC(NC(=O)[C@H]1CN(c2ccc(C(F)(F)F)c3ncccc23)C[C@@H](C)O1)C1CN(C)CCO1]

The title compound was prepared in analogy to the preparation of Example 3 by using 1-(4-methylmorpholin-2-yl)ethanamine (CAS: 1421603-49-7, Enamine, Cat. #: EN300-112760) instead of (4-methylmorpholin-2-yl)methanamine (compound 3d). Prep-HPLC separation gave two isomers Example 35A (RT: 0.744min, 11.2 mg) and Example 35B (RT: 0.757 min, 6.6 mg) as yellow solids with 25%-45% ACN in H₂O (0.05% HCl) as eluent on Phenomenex Synergi C18 (10 μm, 25×150 mm) column.

Example 35A: MS: calc'd 467 (MH⁺), measured 467 (MH⁺). ¹H NMR(400 MHz, METHANOL-d₄) δ=9.34 (d, J=8.5 Hz, 1H), 9.20 (dd, J=1.4, 5.1 Hz, 1H), 8.39 (d, J=8.2 Hz, 1H), 8.10 (dd, J=5.2, 8.5 Hz, 1H), 7.57 (d, J=8.4 Hz, 1H), 4.63−4.54 (m, 1H), 4.25−4.01 (m, 3H), 3.94−3.67 (m, 3H), 3.56−3.41 (m, 3H), 3.26−3.13 (m, 1H), 3.08−2.82 (m, 3H), 2.94 (s, 1.5H), 2.93 (s, 1.5H), 1.38 (d, J=6.3 Hz, 3H), 1.31 (d, J=5.6 Hz, 1.5H), 1.29 (d, J=6.8 Hz, 1.5H).

Example 35B: MS: calc'd 467 (MH⁺), measured 467 (MH⁺). ¹H NMR(400 MHz, METHANOL-d₄) δ=9.18−9.12 (m, 2H), 8.30 (d, J=8.2 Hz, 1H), 7.97 (dd, J=5.0, 8.5 Hz, 1H), 7.48 (d, J=8.2 Hz, 1H), 4.64−4.49 (m, 1H), 4.26−4.04 (m, 3H), 3.91−3.69 (m, 3H), 3.60−3.40 (m, 3H), 3.21−3.09 (m, 1H), 3.00−2.76 (m, 3H), 2.91 (s, 1.5H), 2.90 (s, 1.5H), 1.38 (d, J=6.3 Hz, 3H), 1.30 (d, J=6.9 Hz, 1.5H), 1.28 (d, J=6.9 Hz, 1.5H).

### Example 36

2,7-diazaspiro[4.4]nonan-2-yl-[2R,6R)-6-methyl-4-[8-(trifluoromethyl)-5-quinolyl]morpholin-2-yl]methanone

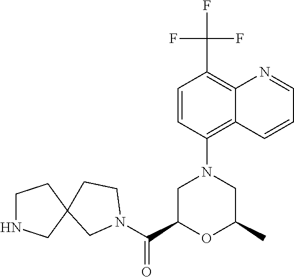 [SMILES: C[C@@H]1CN(c2ccc(C(F)(F)F)c3ncccc23)C[C@H](C(=O)N2CCC3(CCNC3)C2)O1]

The title compound was prepared in analogy to the preparation of Example 7 by using tert-butyl 2,7-diazaspiro[4.4]nonane-2-carboxylate instead of tert-butyl N-(azetidin-3-yl)carbamate (compound 7a). Example 36 (32 mg) was obtained as yellow solid. MS: calc'd 449 (MH⁺), measured 449 (MH⁺). ¹H NMR (400 MHz, METHANOL-d₄) δ=9.62−9.50 (m, 1H), 9.28 (d, J=4.5 Hz, 1H), 8.49 (d, J=8.3 Hz, 1H), 8.30−8.21 (m, 1H), 7.64 (d, J=8.2 Hz, 1H), 4.99−4.80 (m, 1H), 4.28−4.27 (m, 1H), 4.05−3.84 (m, 2H), 3.75−3.38 (m, 8H), 3.29−3.18 (m, 1H), 2.96−2.84 (m, 1H), 2.28−1.95 (m, 4H), 1.37−1.30 (m, 3H).

### Example 37

(2R,6R)-N-(3-azabicyclo[3.3.1]nonan-7-yl)-6-methyl-4-[8-(trifluoromethyl)-5-quinolyl]morpholine-2-carboxamide

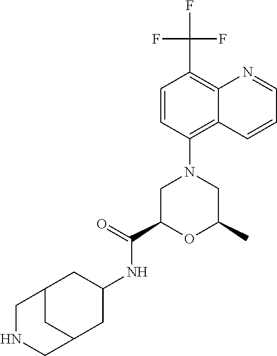 [SMILES: C[C@@H]1CN(c2ccc(C(F)(F)F)c3ncccc23)C[C@H](C(=O)NC2CC3CNCC(C3)C2)O1]

The title compound was prepared according to the following scheme .

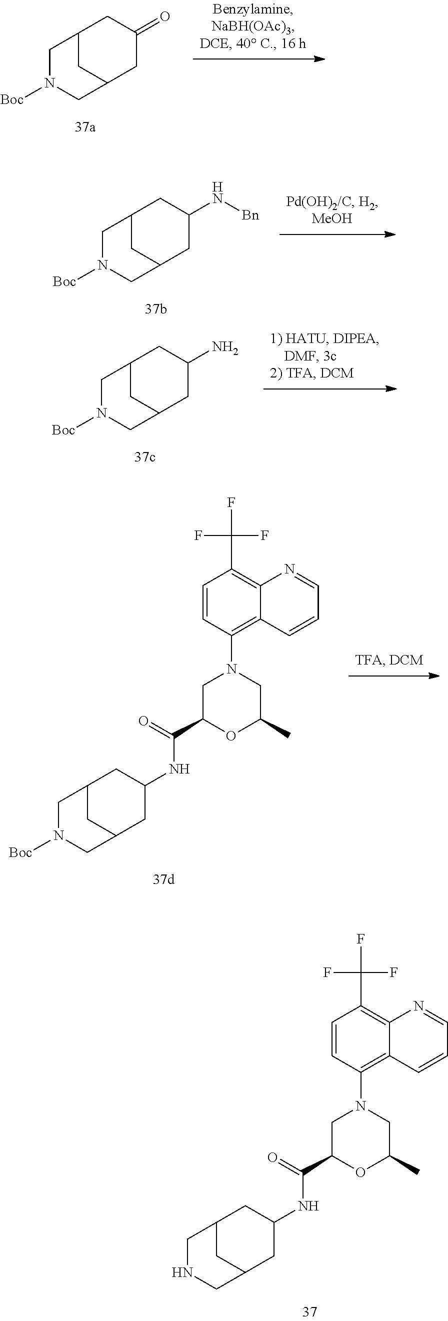 [SMILES: CC(C)(C)OC(=O)N1CC2CC(=O)CC(C2)C1 ; CC(C)(C)OC(=O)N1CC2CC(CC(NCc3ccccc3)C2)C1 ; CC(C)(C)OC(=O)N1CC2CC(N)CC(C2)C1 ; C[C@@H]1CN(c2ccc(C(F)(F)F)c3ncccc23)C[C@H](C(=O)NC2CC3CC(C2)CN(C(=O)OC(C)(C)C)C3)O1 ; C[C@@H]1CN(c2ccc(C(F)(F)F)c3ncccc23)C[C@H](C(=O)NC2CC3CNCC(C3)C2)O1]

Step 1: Preparation of tert-butyl 7-(benzylamino)-3-azabicyclo[3.3.1]nonane-3-carboxylate (Compound 37b)

To a solution of tert-butyl 7-oxo-3-azabicyclo[3.3.1]nonane-3-carboxylate (compound 37a, 250 mg, 1.04 mmol) and benzylamine (0.11 mL, 1.04 mmol) in DCE (3 mL) was added NaBH(OAc)₃ (443 mg, 2.09 mmol) at rt. The reaction mixture was stirred at 40° C. for 16 h, then quenched with aq. NH₄Cl, diluted with H₂O (60 mL), and extracted with DCM (30 mL) three times. The combined organic layer was washed with brine, dried over Na₂SO₄, concentrated and purified by column chromatography (DCM/MeOH=20/1) to give compound 37b (280 mg) as a colorless oil. MS: calc'd 331 (MH⁺), measured 331 (MH⁺).

Step 2: preparation of tert-butyl 7-amino-3-azabicyclo[3.3.1]nonane-3-carboxylate (Compound 37c)

To a solution of tert-butyl 7-(methylamino)-3-azabicyclo[3.3.1]nonane-3-carboxylate (compound 37b, 280.0 mg, 0.85 mmol)) in MeOH (5 mL) was added Pd(OH)₂ on carbon (50.0 mg, 20%, wet). The mixture was charged with H₂ balloon and stirred for 16h at rt, then filtered. The filtrate was concentrated to give crude compound 37c (180 mg) as a colorless oil which was directly used in next step.

Step 3: preparation of tert-butyl 7-[[(2R,6R)-6-methyl-4-[8-(trifluoromethyl)-5-quinolyl]morpholine-2-carbonyl]amino]-3-azabicyclo[3.3.1]nonane-3-carboxylate (Compound 37d)

To a solution of (2R,6R)-6-methyl-4-[8-(trifluoromethyl)-5-quinolyl]morpholine-2-carboxylic acid (compound 3c, 40 mg, 0.12 mmol), tert-butyl 7-amino-3-azabicyclo[3.3.1]nonane-3-carboxylate (compound 37c, 28 mg, 0.12 mmol) and DIPEA (0.05 mL, 0.29 mmol) in DMF (2 mL) was added HATU (54 mg, 0.14 mmol) at rt. The reaction mixture was stirred at rt for 2 h, then diluted with water (50 mL), extracted EtOAc (20 mL) three times. The combined organic layer was washed with brine, dried over Na₂SO₄, and concentrated. The residue was purified by prep-TLC (DCM/MeOH=20/1) to give compound 37d (30 mg) as an orange oil. MS: calc'd 563 (MH⁺), measured 563 (MH⁺).

Step 4: preparation of (2R,6R)-N-(3-azabicyclo[3.3.1]nonan-7-yl)-6-methyl-4-[8-(trifluoromethyl)-5-quinolyl]morpholine-2-carboxamide (Example 37)

To a solution of tert-butyl 7-[[(2R,6R)-6-methyl-4-[8-(trifluoromethyl)-5-quinolyl]morpholine-2-carbonyl]amino]-3-azabicyclo[3.3.1]nonane-3-carboxylate (compound 37d, 30 mg, 0.05 mmol) in DCM (3 mL) was added TFA (1.5 mL) at 0° C. The mixture was stirred at rt for 1.5h. The mixture was concentrated, and the residue was purified by prep-HPLC (FA) to give Example 37 (19 mg) as a yellow solid. MS: calc'd 463 (MET), measured 463 (MH⁺). ¹H NMR (400 MHz, METHANOL-d₄) δ=8.97 (dd, J=1.6, 4.3 Hz, 1H), 8.71 (dd, J=1.7, 8.6 Hz, 1H), 8.53 (brs, 1H), 8.07 (d, J=8.0 Hz, 1H), 7.65 (dd, J=4.2, 8.6 Hz, 1H), 7.27 (d, J=8.0 Hz, 1H), 4.49 (dd, J=2.6, 10.6 Hz, 1H), 4.24−4.09 (m, 2H), 3.67 (d, J=11.8 Hz, 1H), 3.40−3.31 (m, 1H), 3.23−3.13 (m, 2H), 3.13−3.02 (m, 2H), 2.81−2.65 (m, 2H), 2.44−2.25 (m, 4H), 1.98−1.88 (m, 1H), 1.59−1.44 (m, 3H), 1.36 (d, J=6.3 Hz, 3H).

### Example 38

(2R,6R)-4-(8-iodo-5-quinolyl)-6-methyl-N-(1-methyl-4-piperidyl)morpholine-2-carboxamide

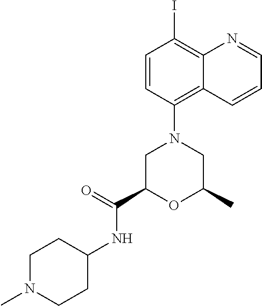 [SMILES: C[C@@H]1CN(c2ccc(I)c3ncccc23)C[C@H](C(=O)NC2CCN(C)CC2)O1]

The title compound was prepared according to the following scheme:

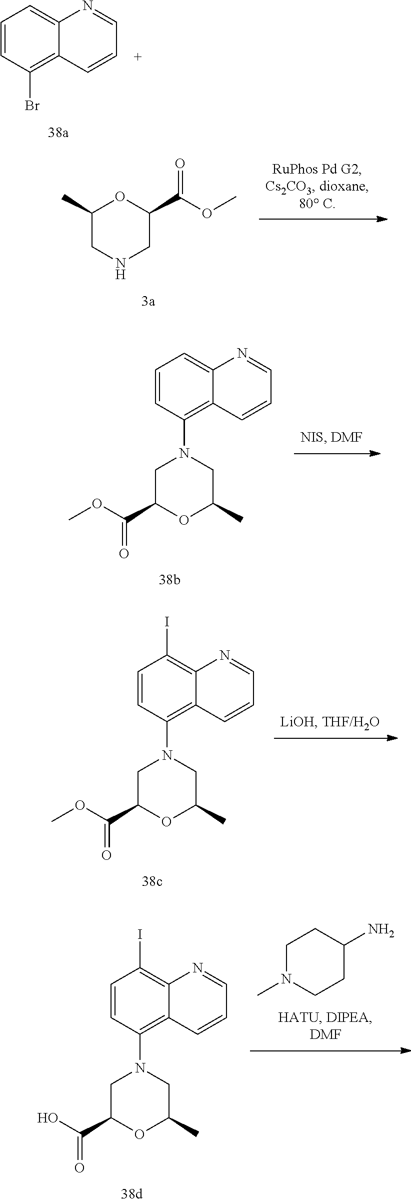

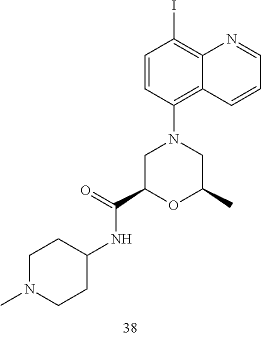 [SMILES: C[C@@H]1CN(c2ccc(I)c3ncccc23)C[C@H](C(=O)NC2CCN(C)CC2)O1]

Step 1: Preparation of methyl (2R,6R)-6-methyl-4-(5-quinolyl)morpholine-2-carboxylate (compound 38b)

To a mixture of methyl (2R,6R)-6-methylmorpholine-2-carboxylate (compound 3a, 99 mg, 0.51 mmol) and 5-bromoquinoline (compound 46a, 100 mg, 0.48 mmol) in 1,4-dioxane (1 mL) was added Cs₂CO₃ (627 mg, 1.92 mmol) and Ruphos Pd-G2 (19 mg, 0.02 mmol). The mixture was stirred at 80° C. for 4 h under N₂, then diluted with EtOAc (80 mL), washed with water (50 mL), brine (50 mL), dried over anhydrous Na₂SO₄ and concentrated to give the crude product which was purified by prep-TLC (PE: EtOAc=2:1) and afforded compound 38b (80 mg) as a yellow oil.

Step 2: Preparation of methyl (2R,6R)-4-(8-iodo-5-quinolyl)-6-methyl-morpholine-2-carboxylate (Compound 38c)

To a solution of methyl (2R,6R)-6-methyl-4-(5-quinolyl)morpholine-2-carboxylate (compound 38b, 80 mg, 0.28 mmol) in DMF (2 mL) was added NIS (75 mg, 0.33 mmol) slowly at 0° C. After addition, the resulting mixture was stirred at 10° C. for 48 h, then partitioned between EtOAc (80 mL) and water (50 mL). The organic phase was separated, washed with brine, dried over anhydrous Na₂SO₄, filtered and concentrated under reduced pressure to give a crude product which was purified by prep-TLC (PE: EtOAc=3:1) to give compound 38c (90 mg) as a yellow oil.

Step 3: Preparation of (2R,6R)-4-(8-iodo-5-quinolyl)-6-methyl-morpholine-2-carboxylic Acid (Compound 38d)

To a solution of methyl (2R,6R)-4-(8-iodo-5-quinolyl)-6-methyl-morpholine-2-carboxylate (compound 38c, 90 mg, 0.22 mmol) in THF (2 mL) was added LiOH.H₂O (10 mg, 0.24 mmol) in water (1 mL). The mixture was stirred at 0° C. for 3 h, then concentrated under reduced pressure to give compound 38d (90 mg, crude) as a white solid which was used directly for next step.

Step 4: Preparation of (2R,6R)-4-(8-iodo-5-quinolyl)-6-methyl-N-(1-methyl-4-piperidyl)morpholine-2-carboxamide (Example 38)

To a solution of (2R,6R)-4-(8-iodo-5-quinolyl)-6-methyl-morpholine-2-carboxylic acid (compound 38d, 45 mg, 0.11 mmol), 1-methylpiperidin-4-amine (CAS: 41838-46-4, 16 mg, 0.14 mmol), and DIPEA (44 mg, 0.34 mmol) in DMF (2 mL) was added HATU (43 mg, 0.11 mmol). The reaction mixture was stirred at rt for 16 h, then partitioned between EtOAc (80 mL) and water (30 mL). The organic phase was separated, washed with brine, dried over anhydrous Na₂SO₄, and concentrated under reduced pressure to give a crude product which was purified by prep-HPLC (FA condition) and afforded Example 38 (10.5 mg) as a yellow solid. MS: calc'd 495 (MH⁺), measured 495 (MH⁺). ¹H NMR (400 MHz, METHANOL-d₄) δ=8.89 (dd, J=1.5, 4.2 Hz, 1H), 8.63 (dd, J=1.5, 8.6 Hz, 1H), 8.30 (d, J=8.1 Hz, 1H), 7.58 (dd, J=4.2, 8.6 Hz, 1H), 7.03 (d, J=8.1 Hz, 1H), 4.46 (dd, J=2.5, 10.5 Hz, 1H), 4.18−4.08 (m, 1H), 4.05−3.94 (m, 1H), 3.56−3.43 (m, 3H), 3.23 (d, J=12.0 Hz, 1H), 3.15−3.00 (m, 2H), 2.83 (s, 3H), 2.77−2.60 (m, 2H), 2.15−2.02 (m, 2H), 1.99−1.83 (m, 2H), 1.33 (d, J=6.4 Hz, 3H).

### Example 39

(2R,6R)-4-(8-iodo-5-quinolyl)-6-methyl-N-[(4-methylmorpholin-2-yl)methyl]morpholine-2-carboxamide

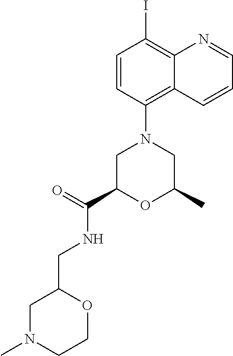 [SMILES: C[C@@H]1CN(c2ccc(I)c3ncccc23)C[C@H](C(=O)NCC2CN(C)CCO2)O1]

The title compound was prepared in analogy to the preparation of Example 38 by using (4-methylmorpholin-2-yl)methanamine (compound 3d) instead of 1-methylpiperidin-4-amine. Example 39 (10.4 mg) was obtained as a yellow solid. MS: calc'd 511 (MH⁺), measured 511 (MH⁺). ¹H NMR (400 MHz, METHANOL-d₄) δ−8.90 (dd, J=1.6, 4.3 Hz, 1H), 8.64 (dd, J=1.6, 8.4 Hz, 1H), 8.30 (d, J=8.1 Hz, 1H), 7.58 (dd, J=4.3, 8.4 Hz, 1H), 7.04 (d, J=8.1 Hz, 1H), 4.48 (dd, J=2.6, 10.6 Hz, 1H), 4.18−4.09 (m, 1H), 3.99 (d, J=12.0 Hz, 1H), 3.75−3.65 (m, 2H), 3.55 (d, J=11.5 Hz, 1H), 3.46−3.35 (m, 2H), 3.23 (d, J=12.0 Hz, 1H), 3.06−2.93 (m, 2H), 2.75−2.62 (m, 2H), 2.53 (s. 3H), 2.52−2.44 (m, 1H), 2.30−2.18 (m, 1H), 1.33 (d, J=6.4 Hz, 3H).

### Example 40

cis-(2R,6R)-N-[4-fluoropyrrolidin-3-yl]-4-(8-iodo-5-quinolyl)-6-methyl-morpholine-2-carboxamide (mixture of two cis diastereomers at the marked positions with *)

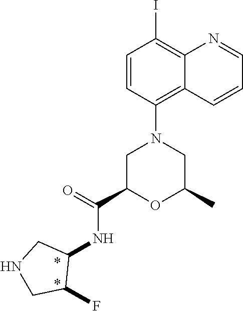

The title compound was prepared according to the following scheme:

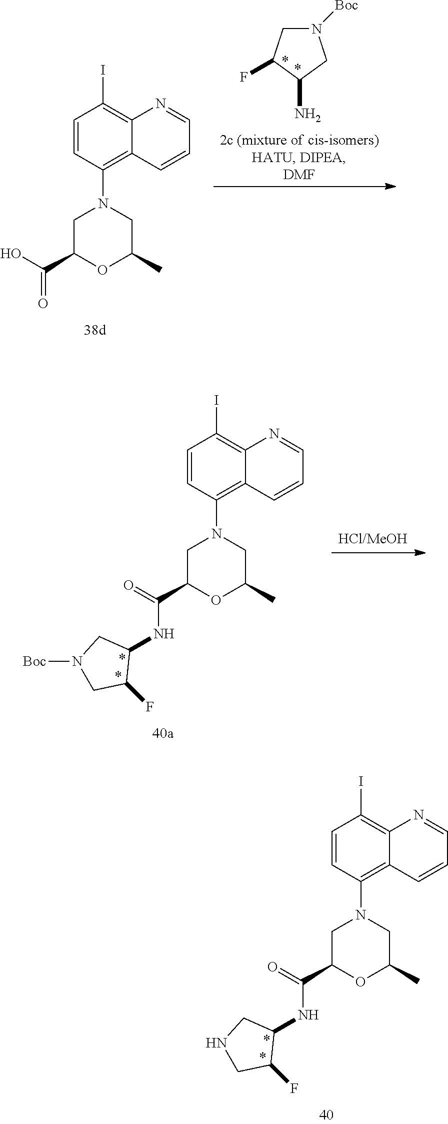

Step 1: preparation of cis-tert-butyl 3-fluoro-4-[[(2R,6R)-4-(8-iodo-5-quinolyl)-6-methyl-morpholine-2-carbonyl]amino]pyrrolidine-1-carboxylate (Compound 40a)

To a solution of (2R,6R)-4-(8-iodo-5-quinolyl)-6-methyl-morpholine-2-carboxylic acid (compound 38d, 58 mg, 0.15 mmol), cis-tert-butyl 3-amino-4-fluoro-pyrrolidine-1-carboxylate (compound 2c, 33 mg, 0.16 mmol) and DIPEA (0.06 mL, 0.36 mmol) in DMF (2 mL) was added HATU (66 mg, 0.17 mmol). The reaction mixture was stirred at rt for 2 h, then water (10 mL) was added. The solid was filtered and dried to give the desired product compound 40a (35 mg) as a white solid.

Step 2: Preparation of cis-(2R,6R)-N-[4-fluoropyrrolidin-3-yl]-4-(8-iodo-5-quinolyl)-6-methyl-morpholine-2-carboxamide

To a solution of cis-tert-butyl 3-fluoro-4-[[(2R,6R)-4-(8-iodo-5-quinolyl)-6-methyl-morpholine-2-carbonyl]amino]pyrrolidine-1-carboxylate (compound 40a, 35 mg, 0.06 mmol) in MeOH (1 mL) was added HCl solution (4 M in MeOH, 1.0 mL, 4.0 mmol). The reaction mixture was stirred at 20° C. for 1 h, then solvent was removed under reduced pressure and the residue was purified by prep-HPLC (HCl) to give Example 40 (8 mg) as a white solid. MS: calc'd 485 (MH⁺), measured 485 (MH⁺). ¹H NMR (400 MHz, DMSO-d₆) δ=9.64−9.52 (brs, 1H), 9.30−9.20 (brs, 1H), 8.98−8.97 (m, 1H), 8.56 (d, J=8.0 Hz, 1H), 8.31 (d, J=8.0 Hz, 1H), 7.64 (dd, J=4.0, 8.0 Hz, 1H), 7.06 (dd, J=3.6, 8.4Hz, 1H), 5.37−5.12 (m, 1H), 4.51−4.47 (m, 2H), 4.15−4.05 (m, 1H), 3.66−3.50 (m, 6H), 2.68−2.53 (m, 2H), 1.26 (d, J=6.4 Hz, 3H).

### Example 41

cis-(2R,6R)-4-(8-bromo-5-quinolyl)-N-[4-fluoropyrrolidin-3-yl]-6-methyl-morpholine-2-carboxamide (mixture of two cis diastereoisomers at the positions marked with *)

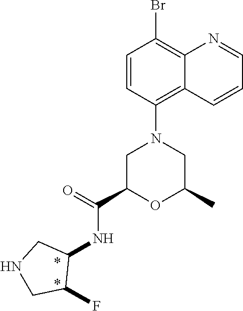

The title compound was prepared according to the following scheme:

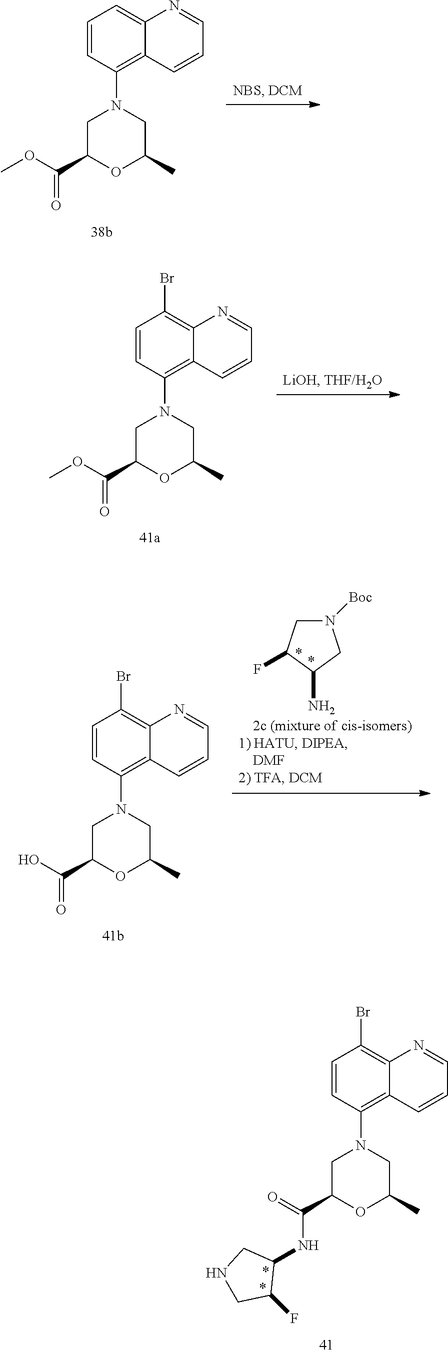

Step 1: Preparation of methyl (2R,6R)-4-(8-bromo-5-quinolyl)-6-methyl-morpholine-2-carboxylate (Compound 41a)

To a solution of methyl (2R,6R)-6-methyl-4-(5-quinolyl)morpholine-2-carboxylate (compound 38b, 115 mg, 0.4 mmol) in DCM (3 mL) was added NBS (107 mg, 0.60 mmol) at 0° C. The mixture was warmed to rt and stirred for 3 h, then diluted with DCM (30 mL), washed with sat NaHCO₃ and brine. The organic layer was dried over Na₂SO₄ and concentrated. The residue was purified by column chromatography to give compound 50a (140 mg) as an oil.

Step 2: Preparation of (2R,6R)-4-(8-bromo-5-quinolyl)-6-methyl-morpholine-2-carboxylic Acid (Compound 41b)

To a solution of methyl (2R,6R)-4-(8-bromo-5-quinolyl)-6-methyl-morpholine-2-carboxylate (compound 41a, 140 mg, 0.38 mmol) in THF (2 mL) was added aq. LiOH (2 M, 2 mL, 4 mmol). The mixture was stirred at rt overnight, then diluted with water, and extracted with EtOAc. The organic layer was removed, and the water layer was adjusted to pH around 2 and extracted with DCM. The DCM phase was dried over Na₂SO₄ and concentrated to give a brown oil (80 mg) which was directly used in next step. MS: calc'd 351 and 353 (MH⁺), measured 351 and 353 (MH⁺).

Step 3: Preparation of cis-(2R,6R)-4-(8-bromo-5-quinolyl)-N-14-fluoropyrrolidin-3-y11-6-methyl-morpholine-2-carboxamide (Example 41)

To a solution of (2R,6R)-4-(8-bromo-5-quinolyl)-6-methyl-morpholine-2-carboxylic acid (compound 41b, 39 mg, 0.11 mmol), cis-tort-butyl 3-amino-4-fluoro-pyrrolidine-1-carboxylate (CAS: 1431720-86-3, compound 2c, 45 mg, 0.22 mmol) and DIPEA (57 mg, 77 μL, 0.44 mmol) in DCM (3 mL) was added HATU (63 mg, 0.16 mmol). The reaction mixture was stirred at rt overnight, then diluted with DCM, washed with sat NH₄Cl and brine, dried over Na₂SO₄ and concentrated. The residue was dissolved in DCM (2 mL) and cooled with ice water bath, then TFA (1 mL) was added drop-wise. The reaction mixture was stirred at rt for 1 h, then concentrated to give a crude product which was purified by prep-HPLC to give Example 41 (19 mg) as a light brown solid. MS: calc'd 437 and 439 (MH⁺), measured 437 and 439 (MH⁺). ¹H NMR (400 MHz, METHANOL-d₄) δ=8.93 (dd, J=1.7, 4.2 Hz, 1H), 8.72 (dd, J=1.6, 8.6 Hz, 1H), 8.03 (d, J=8.2 Hz, 1H), 7.63 (dd, J=4.2, 8.5 Hz, 1H), 7.17 (dd, J=3.1, 8.1 Hz, 1H), 5.41−5.20 (m, 1H), 4.82−4.65 (m, 1H), 4.60−4.51 (m, 1H), 4.21−4.09 (m, 1H), 3.80−3.49 (m, 4H), 3.43 (dt, J=4.2, 11.2 Hz, 1H), 3.27−3.19 (m, 1H), 2.82−2.72 (m, 1H), 2.71−2.59 (m, 1H), 1.33 (d, J=6.2 Hz, 3H).

### Example 42

(2R,6R)-4-(8-bromo-5-quinolyl)-6-methyl-N-(1-methyl-4-piperidyl)morpholine-2-carboxamide

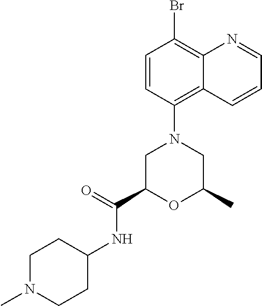 [SMILES: C[C@@H]1CN(c2ccc(Br)c3ncccc23)C[C@H](C(=O)NC2CCN(C)CC2)O1]

The title compound was prepared according to the following scheme:

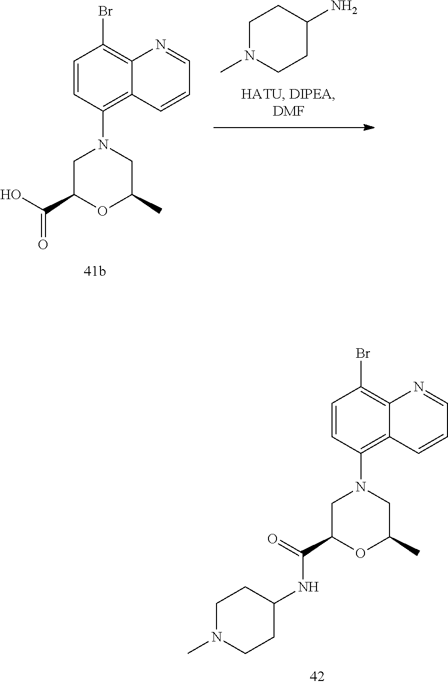

To a solution of (2R,6R)-4-(8-bromo-5-quinolyl)-6-methyl-morpholine-2-carboxylic acid (compound 41b, 39 mg, 0.11 mmol), 1-methylpiperidin-4-amine (25 mg, 28 μl, 0.22 mmol) and DIPEA (57 mg, 77 μl, 0.44 mmol) in DCM (3 mL) was added HATU (63 mg, 0.16 mmol). The mixture was stirred at rt overnight, then diluted with DCM, washed with sat NH₄Cl and brine, dried over Na₂SO₄ and concentrated to give a crude product which was purified by prep-HPLC to give Example 42 (13 mg) as a light yellow powder. MS: calc'd 447 and 449 (MH⁺), measured 447 and 449 (MH⁺). ¹H NMR (400 MHz, METHANOL-d₄) δ=8.95 (dd, J=1.7, 4.2 Hz, 1H), 8.74 (dd, J=1.7, 8.5 Hz, 1H), 8.05 (d, J=8.2 Hz, 1H), 7.64 (dd, J=4.2, 8.5 Hz, 1H), 7.18 (d, J=8.2 Hz, 1H), 4.48 (dd, J=2.6, 10.6 Hz, 1H), 4.22−4.09 (m, 1H), 3.95−3.83 (m, 1H), 3.58−3.51 (m, 1H), 3.27−3.15 (m, 3H), 2.79−2.61 (m, 4H), 2.59 (s, 3H), 2.08−1.93 (m, 2H), 1.87−1.71 (m, 2H), 1.35 (d, J=6.2 Hz, 3H).

### Example 43

(2R,6R)-4-(8-ethynyl-5-quinolyl)-6-methyl-N-(1-methyl-4-piperidyl)morpholine-2-carboxamide

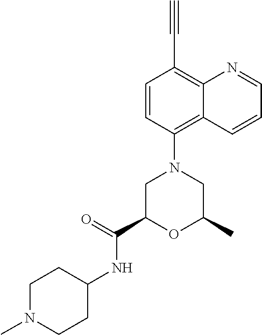 [SMILES: C#Cc1ccc(N2C[C@@H](C)O[C@@H](C(=O)NC3CCN(C)CC3)C2)c2cccnc12]

The title compound was prepared according to the following scheme:

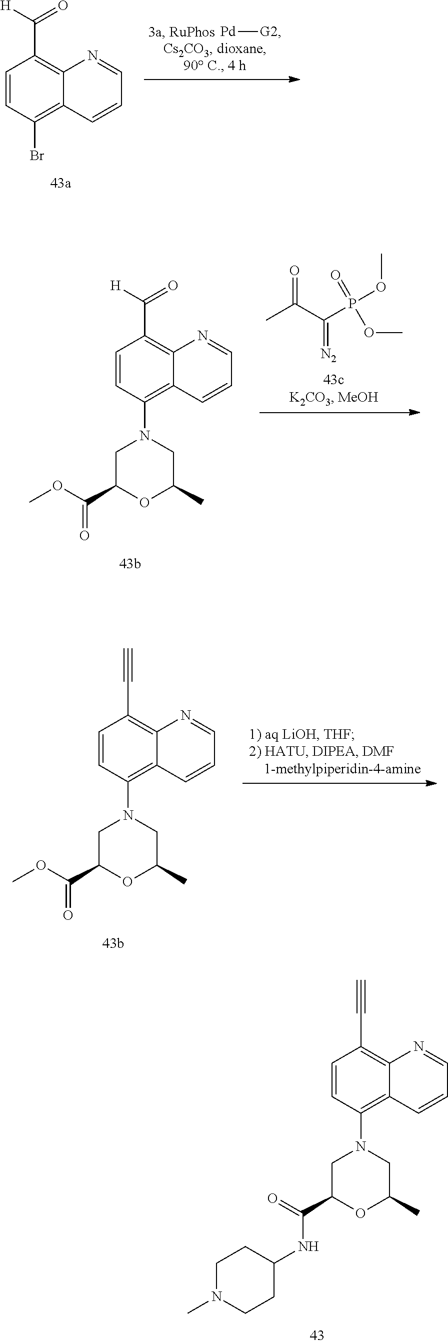

Step 1: Preparation of methyl (2R,6R)-4-(8-formyl-5-quinolyl)-6-methyl-morpholine-2-carboxylate (Compound 43b)

To a solution of 5-bromoquinoline-8-carbaldehyde (CAS: 885267-41-4, compound 43a, 210 mg, 0.89 mmol) in 1,4-dioxane (8 mL) was added methyl (2R,6R)-6-methylmorpholine-2-carboxylate (compound 3a, 174 mg, 0.89 mmol), Cs₂CO₃ (725 mg, 2.2 mmol,) and Ruphos Pd-G2 (69 mg, 0.09 mmol). The reaction mixture was stirred at 90° C. for 4 h under N₂, then diluted with EtOAc and filtered. The filtrate was concentrated to give crude compound 43b (321 mg) as a yellow gum. MS: calc'd 315 (MH⁺), measured 315 (MH⁺).

Step 2: Preparation of methyl (2R,6R)-4-(8-ethynyl-5-quinolyl)-6-methyl-morpholine-2-carboxylate (Compound 45d)

To a solution of methyl (2R,6R)-4-(8-formyl-5-quinolyl)-6-methyl-morpholine-2-carboxylate (compound 43b, 290 mg, 0.92 mmol) in MeOH (10 mL) was added 1-diazo-1-dimethoxyphosphoryl-propan-2-one (compound 43c, 443 mg, 2.3 mmol) and K₂CO₃ (382 mg, 2.8 mmol). The reaction mixture was stirred at rt for 4 h, then water (80 mL) was added, extracted with EtOAc (80 mL). The organic layer was dried over Na₂SO₄ and concentrated. The residue was purified by prep-TLC (EA: PE=1:2) to give compound 43d (103 mg) as a yellow gum. MS: calc'd 311 (MH⁺), measured 311 (MH⁺).

Step 3: Preparation of (2R,6R)-4-(8-ethynyl-5-quinolyl)-6-methyl-N-(1-methyl-4-piperidyl)morpholine-2-carboxamide (Example 43)

To a solution of methyl (2R,6R)-4-(8-ethynyl-5-quinolyl)-6-methyl-morpholine-2-carboxylate (compound 43d, 103 mg, 0.33 mmol) in THF (5 mL) was added LiOH.H₂O (14 mg, 0.33 mmol) in water (5 mL). The mixture was stirred at rt for 2 h, then water (50 mL) was added. Then the mixture was adjusted to pH around 3 with aq. HCl (1 N) and extracted with DCM. The organic layer was dried over Na₂SO₄ and concentrated to give a crude product (70 mg) as a yellow solid. MS: calc'd 297 (MH⁺), measured 297 (MH⁺).

To a solution of the crude product obtained above (40 mg, 0.13 mmol) and 1-methylpiperidin-4-amine (19 mg, 0.16 mmol) in DMF (3 mL) was added DIPEA (0.07 mL, 0.40 mmol) and HATU (62 mg, 0.16 mmol). The reaction mixture was stirred at rt for 2 h, then concentrated and purified by prep-HPLC (FA) to give Example 43 (19 mg) as a yellow solid. MS: calc'd 393 (MH⁺), measured 393 (MH⁺). ¹H NMR (400 MHz, METHANOL-d₄) δ=8.92 (dd, J=1.6, 4.3 Hz, 1H), 8.71 (dd, J=1.6, 8.5 Hz, 1H), 8.49 (s, 1H), 7.92 (d, J=7.9 Hz, 1H), 7.62 (dd, J=4.3, 8.5 Hz, 1H), 7.23 (d, J=7.9 Hz, 1H), 4.49 (dd, J=2.6, 10.6 Hz, 1H), 4.17−4.13 (m, 10.0 Hz, 1H), 4.01−3.92 (m, 1H), 3.86 (s, 1H), 3.59 (d, J=11.8 Hz, 1H), 3.38−3.34 (m, 2H), 3.29 (d, J=12.0 Hz, 1H), 2.90 (t, J=8.0, 2H), 2.79'2.72 (m, 1H), 2.75 (s, 3H), 2.69 (dd, J=10.3, 11.8 Hz, 1H), 2.11−2.01 (m, 2H), 1.94−1.81 (m, 2H), 1.36 (d, J=6.3 Hz, 3H).

### Example 44

(2R,6R)-N-[1R,4R)-2-azabicyclo[2.2.1]heptan-5-yl]-6-methyl-4-[8-(trifluoromethyl)-5-quinolyl]morpholine-2-carboxamide

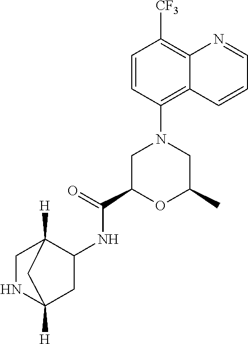 [SMILES: C[C@@H]1CN(c2ccc(C(F)(F)F)c3ncccc23)C[C@H](C(=O)NC2C[C@H]3C[C@@H]2CN3)O1]

The title compound was prepared according to the following scheme:

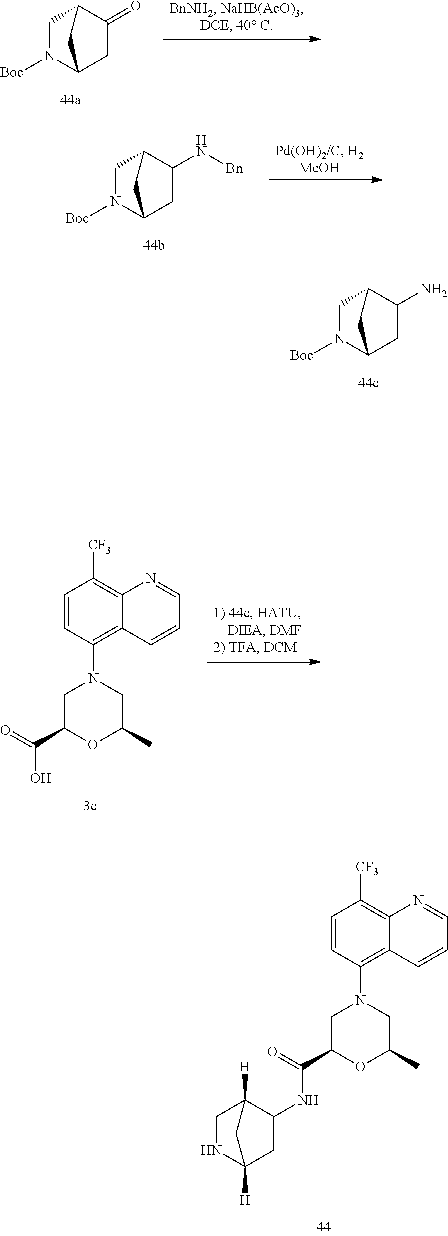

Step 1: Preparation of tert-butyl (1R,4R)-5-(benzylamino)-2-azabicyclo[2.2.1]heptane-2-carboxylate (Compound 44b)

To a solution of tert-butyl (1R,4R)-5-oxo-2-azabicyclo[2.2.1]heptane-2-carboxylate (CAS: 1400808-00-5, Wuxi AppTech, Cat. #: WX120461, compound 44a, 450 mg, 2.2 mmol) and benzylamine (342 mg, 2.9 mmol) in DCE (20 mL) was added NaHB(OAc)₃ (2.28 g, 10.8 mmol). The mixture was stirred at 40° C. for 16 h, then diluted with water (50 mL), and extracted with DCM. The organic phase was washed with brine, dried over anhydrous Na₂SO₄, filtered and concentrated to give the crude product which was purified by prep-TLC (EA/PE=1/2) to give compound 44b (280 mg) as a white solid.

Step 2: Preparation of tert-butyl (1R,4R)-5-amino-2-azabicyclo[2.2.1]heptane-2-carboxylate (Compound 44c)

To a solution of tert-butyl (1R,4R)-5-(benzylamino)-2-azabicyclo[2.2.1]heptane-2-carboxylate (compound 44b, 280 mg, 0.93 mmol) in MeOH (30 mL) was added Pd/C (100 mg, 10%, wet). The mixture was degassed with H₂ for 3 times and stirred at rt for 16 h under hydrogen balloon. The reaction mixture was then filtered, and the filtrate was concentrated to give crude compound 44c (200 mg) as a white solid which was used directly in the next step. MS: calc'd 213 (MH⁺), measured 157 (MH⁺-55).

Step 3: Preparation of (2R,6R)-N-R1R,4R)-2-azabicyclo[2.2.11heptan-5-yl]-6-methyl-4-[8-(trifluoromethyl)-5- quinolyl]morpholine-2-carboxamide (Example 44)

To a solution of (2R,6R)-6-methyl-4-[8-(trifluoromethyl)-5-quinolyl]morpholine-2-carboxylic acid (compound 3c, 40 mg, 0.12 mmol) and tert-butyl (1R,4R)-5-amino-2-azabicyclo[2.2.1]heptane-2-carboxylate (compound 44c, 30 mg, 0.14 mmol) in DMF (5 mL) was added DIPEA (46 mg, 0.35 mmol) and HATU (34 mg, 0.14 mmol). The reaction mixture was stirred at rt for 16 h, then diluted with water, and extracted with EtOAc. The organic phase was washed with brine, dried over anhydrous Na₂SO₄, filtered and concentrated. The residue was purified by prep-TLC (EA) to give the desired product (30 mg) as a colorless oil which was dissolved in DCM (5 mL), and treated with TFA (2 mL). The mixture was stirred at rt for 2 h, then concentrated. The residue was purified by prep-HPLC (HCl) to afford Example 44 (7.1 mg) as a yellow solid. MS: calc'd 435 (MH⁺), measured 435 (MH⁺). ¹H NMR (400 MHz, METHANOL-d₄) δ=9.16−9.11 (m, 2H), 8.28 (d, J=8.4 Hz, 1H), 7.95 (dd, J=5.2, 8.8 Hz, 1H), 7.45 (d, J=8.0 Hz, 1H), 4.59 (dd, J=2.4, 10.8 Hz, 1H), 4.32−4.25 (m, 1H), 4.24·4.17 (m, 1H), 4.11−4.03 (m, 1H), 3.71 (d, J=12.0 Hz, 1H), 3.41 (t, J=11.9 Hz, 2H), 3.17−3.08 (m, 1H), 2.96 (brs, 1H), 2.93−2.85 (m, 1H), 2.80 (dd, J=10.4, 11.2 Hz, 1H), 2.35−2.23 (m, 1H), 2.01−1.95 (m, 1H), 1.90−1.80 (m, 1H), 1.69 (td, J=3.9, 15.2 Hz, 1H), 1.38 (d, J=6.4 Hz, 3H).

### Example 45

(2R,6R)-N-(3-azabicyclo[3.3.1]nonan-9-yl)-6-methyl-4-[8-(trifluoromethyl)-5-quinolyl]morpholine-2-carboxamide

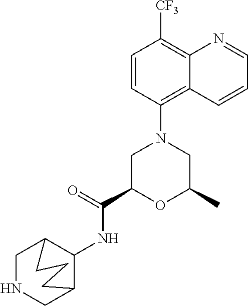 [SMILES: C[C@@H]1CN(c2ccc(C(F)(F)F)c3ncccc23)C[C@H](C(=O)NC2C3CCCC2CNC3)O1]

The title compound was prepared in analogy to the preparation of Example 45 by using tert-butyl 9-oxo-3-azabicyclo[3.3.1]nonane-3-carboxylate (CAS: 512822-34-3) instead of tert-butyl (1R,4R)-5-oxo-2-azabicyclo[2.2.1]heptane-2-carboxylate (compound 44a). Example 45 (12 mg) was obtained as a yellow solid. MS: calc'd 463 (MH⁺), measured 463 (MH⁺). ¹H NMR (400 MHz, METHANOL-d₄) 6 =8.98 (dd, J=1.6, 4.1 Hz, 1H), 8.72 (dd, J=1.7, 8.6 Hz, 1H), 8.52 (s, 1H), 8.08 (d, J=8.0 Hz, 1H), 7.65 (dd, J=4.2, 8.6 Hz, 1H), 7.30 (d, J=8.0 Hz, 1H), 4.59 (dd, J=2.5, 10.7 Hz, 1H), 4.27−4.18 (m, 1H), 4.16−4.10 (m, 1H), 3.66 (d, J=11.9 Hz, 1H), 3.52 (d, J=13.6 Hz, 2H), 3.42 (d, J=17.6 Hz, 2H), 3.38−3.34 (m, 1H), 2.85 (t, J=11 .3 Hz, 1H), 2.74 (dd, J=10.4, 12.0 Hz, 1H), 2.25—2.16 (m, 2H), 2.03—1.76 (m, 6H), 1.37 (d, J=6.3 Hz, 3H).

### Example 46

(2R,6R)-6-methyl-N-(3-oxa-7-azabicyclo[3.3.1]nonan-9-yl)-4-[8-(trifluoromethyl)-5-quinolyl]morpholine-2-carboxamide

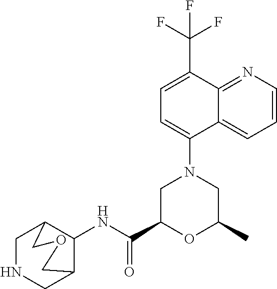 [SMILES: C[C@@H]1CN(c2ccc(C(F)(F)F)c3ncccc23)C[C@H](C(=O)NC2C3CNCC2COC3)O1]

The title compound was prepared in analogy to the preparation of Example 7 by using tert-butyl 9-amino-3-oxa-7-azabicyclo [3.3. 1]nonane-7-carboxylate (CAS: 1251015-74-3, Wuxi AppTec, Cat. #: WX120123) instead of tert-butyl N-(azetidin-3-yl)carbamate (compound 7a). Prep-HPLC separation gave two single isomers, Example 46A (RT: 0.711 min, 9.5 mg) and 46B (RT: 0.730 min, 7.4 mg) as yellow solids with 20%˜40% ACN in H₂O (0.05% HCl) as eluent on Phenomenex Synergi C18 (10 μm, 25×150 mm) column.

Example 46A: MS: calc'd 465 (MH⁺), measured 465 (MH⁺). ¹H NMR (400 MHz, METHANOL-d₄) δ=9.44−9.29 (m, 1H), 9.19 (d, J=3.0 Hz, 1H), 8.38 (dd, J=3.8, 7.8 Hz, 1H), 8.14−8.04 (m, 1H), 7.56 (dd, J=3.3, 8.3 Hz, 1H), 4.71 (d, J=10.5 Hz, 1H), 4.29—4.17 (m, 4H), 3.94 (d, J=12.0 Hz, 2H), 3.74 (d, J=11.9 Hz, 1H), 3.61−3.43 (m, 5H), 3.04 (t, J=10.7 Hz, 1H), 2.88 (t, J=11.0 Hz, 1H), 2.30−2.20 (m, 2H), 1.39 (d, J=6.3 Hz, 3H).

Example 46B: MS: calc'd 465 (MH⁺), measured 465 (MH⁺). ¹H NMR (400 MHz, METHANOL-d₄) δ=9.21 (d, J=8.7 Hz, 1H), 9.08 (d, J=4.8 Hz, 1H), 8.26 (d, J=8.0 Hz, 1H), 8.00−7.91 (m, 1H), 7.43 (d, J=8.2 Hz, 1H), 4.57 (d, J=8.9 Hz, 1H), 4.20−4.07 (m, 2H), 4.01−3.88 (m, 4H), 3.67−3.54 (m, 3H), 3.44−3.32 (m, 3H), 2.90 (t, J=11.3 Hz, 1H), 2.76 (t, J=11.3 Hz, 1H), 2.14−2.05 (m, 2H), 1.28 (d, J=6.2 Hz, 3H).

### Example 47

(2R,6R)-N-(8-azabicyclo[3.2.1]octan-3-yl)-6-methyl-4-[8-(trifluoromethyl)-5-quinolyl]morpholine-2-carboxamide

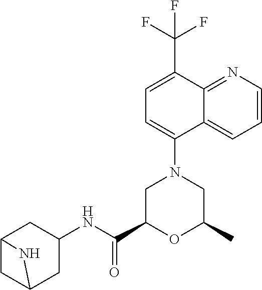 [SMILES: C[C@@H]1CN(c2ccc(C(F)(F)F)c3ncccc23)C[C@H](C(=O)NC2CC3CC(C2)N3)O1]

The title compound was prepared in analogy to the preparation of Example 7 by using tert-butyl 3-amino-8-azabicyclo[3.2.1]octane-8-carboxylate (CAS: 174486-93-2, Wuxi AppTec, Cat. #: WX120356) instead of tert-butyl N-(azetidin-3-yl)carbamate (compound 7a). Prep-HPLC separation gave two single isomers, Example 47A (RT: 0.724min, 3.9 mg) and 47B (RT: 0.735 min, 9.1 mg) as yellow solids with 20%-40% ACN in H₂O (0.05% HCl) as eluent on Phenomenex Synergi C18 (10 μm, 25×150 mm) column.

Example 47A: MS: calc'd 449 (MH⁺), measured 449 (MH⁺). ¹H NMR (400 MHz, METHANOL-d₄) δ=9.19−9.12 (m, 2H), 8.30 (d, J=8.2 Hz, 1H), 7.98 (dd, J=4.9, 8.7 Hz, 1H), 7.47 (d, J=8.2 Hz, 1H), 4.55 (dd, J=2.5, 10.7 Hz, 1H), 4.31 (tt, J=5.8, 11.6 Hz, 1H), 4.25−4.15 (m, 1H), 4.15−4.05 (m, 2H), 3.71 (d, 1=11.8 Hz, 1H), 3.43 (d, J=12.1 Hz, 1H), 2.89 (t, J=11.3 Hz, 1H), 2.85−2.77 (MH⁺), 2.21−1.91 (m, 8H), 1.38 (d, J=6.3 Hz, 3H).

Example 47B: MS: calc'd 449 (MH⁺), measured 449 (MH⁺). ¹H NMR (400 MHz, METHANOL-d₄) δ=9.20 (dd, J=1.3, 8.5 Hz, 1H), 9.07 (dd, J=1.3, 5.0 Hz, 1H), 8.26 (d, J=8.3 Hz, 1H), 7.96 (dd, J=5.1, 8.5 Hz, 1H), 7.42 (d, J=8.2 Hz, 1H), 4.49 (dd, J=2.4, 10.7 Hz, 1H), 4.18−4.08 (m, 1H), 4.05−3.93 (m, 3H), 3.61 (d, J=12.1 Hz, 1H), 3.36 (d, J=12.1 Hz, 1H), 2.85 (J=11.5 Hz, 1H), 2.78−2.68 (m, 1H), 2.27−2.04 (m, 8H), 1.27 (d, J=6.2 Hz, 3H).

### Example 48

(2R,6R)-6-methyl-N-(morpholin-2-ylmethyl)-4-[8-(trifluoromethyl)-5-quinolyl]morpholine-2-carboxamide

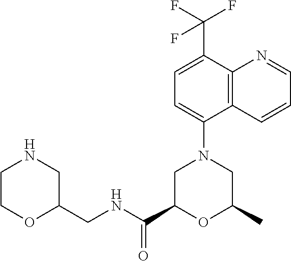 [SMILES: C[C@@H]1CN(c2ccc(C(F)(F)F)c3ncccc23)C[C@H](C(=O)NCC2CNCCO2)O1]

The title compound was prepared in analogy to the preparation of Example 7 by using tert-butyl 2-(aminomethyl)morpholine-4-carboxylate (CAS: 140645-53-0) instead of tert-butyl N-(azetidin-3-yl)carbamate (compound 7a) Example 48 (11.7 mg) was obtained as a yellow solid. MS: calc'd 439 (MH⁺), measured 439 (MH⁺). ¹H NMR (400 MHz, METHANOL-d₄) δ=9.24 (dd, J=1.5, 8.5 Hz, 1H), 9.17 (dd, J=1.5, 5.0 Hz, 1H), 8.34 (d, J=8.3 Hz, 1H), 8.03 (dd, J=5.0, 8.7 Hz, 1H), 7.51 (d, J=8.2 Hz, 1H), 4.60−4.53 (m, 1H), 4.28−4.17 (m, 1H), 4.13 (dd, J=3.3, 13.0 Hz, 1H), 3.95−3.80 (m, 2H), 3.74 (d, J=11.9 Hz, 1H), 3.52−3.40 (m, 3H), 3.39−3.34 (m, 1H), 3.31−3.25 (m, 1H), 3.23−3.13 (m, 1H), 2.98≤2.88 (m, 2H), 2.83 (t, J=11.2 Hz, 1H), 1.38 (d, J=6.3 Hz, 3H).

### Example 49

(2R,6R)-N-(3-azabicyclo[3.2.0]heptan-6-yl)-6-methyl-4-[8-(trifluoromethyl)-5-quinolyl]morpholine-2-carboxamide

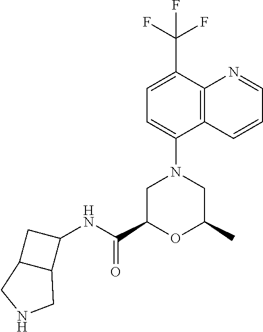 [SMILES: C[C@@H]1CN(c2ccc(C(F)(F)F)c3ncccc23)C[C@H](C(=O)NC2CC3CNCC32)O1]

The title compound was prepared in analogy to the preparation of Example 7 by using tert-butyl 6-amino-3-azabicyclo[3.2.0]heptane-3-carboxylate (CAS: 1250884-66-2, PharmaBlock, Cat. #: PBN20120396) instead of tert-butyl N-(azetidin-3-yl)carbamate (compound 7a). Prep-HPLC separation gave 3 compounds Example 49A (RT: 0.754 min, 6.7 mg), Example 49B (RT: 0.763 min, 8.5 mg) and Example 49C (RT: 0.739 min, 4.0 mg) as yellow solids with 18%˜48% ACN in H₂O (0.1% TFA) as eluent on Phenomenex Synergi C18 (10 μm, 25×150 mm) column.

Example 49A: MS: calc'd 435 (MH⁺), measured 435 (MH⁺) ¹H NMR (400 MHz, METHANOL-d₄) δ=9.00−8.95 (m, 1H), 8.72−8.65 (m, 1H), 8.08 (d, J=8.0 Hz, 1H), 7.68−7.63 (m, 1H), 7.29 (d, J=8.0 Hz, 1H), 4.58−4.52 (m, 1H), 4.40−4.30 (m, 1H), 4.23−4.14 (m, 1H), 3.75−3.67 (m, 1H), 3.48−3.36 (m, 3H), 3.38−3.35 (m, 1H), 3.31−3.01 (m, 3H), 2.86−2.80 (m, 1H), 2.7−2.65 (m, 2H), 2.13−2.07 (m, 1H), 1.37 (d, J=6.4 Hz, 3H).

Example 49B: MS: calc'd 435 (MH⁺), measured 435 (MH⁺). ¹H NMR (400 MHz, METHANOL-d₄) δ=9.03−8.97 (m, 1H), 8.75−8.66 (m, 1H), 8.08 (d, J=8.0 Hz, 1H), 7.70−7.62 (m, 1H), 7.29 (d, J=8.0 Hz, 1H), 4.57 (dd, J=2.4, 10.4, 1H), 4.40−4.30 (m, 1H), 4.24−4.14 (m, 1H), 3.67−3.64 (m, 1H), 3.45−3.40 (m, 3H), 3.38−3.35 (m, 1H), 3.31−3.01 (m, 3H), 2.86−2.80 (m, 1H), 2.74−2.71 (m, 2H), 2.13−2.07 (m, 1H), 1.37 (d, J=6.4 Hz, 3H).

Example 49C: MS: calc'd 435 (MH⁺), measured 435 (MH⁺). ¹H NMR (400 MHz, METHANOL-d₄) δ=8.99−8.94 (m, 1H), 8.75−8.68 (m, 1H), 8.08 (d, J=8.0 Hz, 1H), 7.69−7.60 (m, 1H), 7.28 (d, J=8.0 Hz, 1H), 4.52−4.49 (m, 1H), 4.20−4.14 (m, 2H), 3.66−3.56 (m, 2H), 3.44−3.38 (m, 3H), 3.22−3.19 (m, 3H), 2.79−2.72 (m, 2H), 2.50−2.46 (m, 1H), 2.21−2.18 (m, 1H), 1.38 (d, J=6.4 Hz, 3H).

### Example 50

The following tests were carried out in order to determine the activity of the compounds of formula (I) in HEK293-Blue-hTLR-7/8/9 cells assay.

HEK293-Blue-hTLR-7 Cells Assay:

A stable HEK293-Blue-hTLR-7 cell line was purchased from InvivoGen (Cat. #: hkb-htlr7, San Diego, Calif. USA). These cells were originally designed for studying the stimulation of human TLR7 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR7 cells with TLR7 ligands. Therefore the reporter expression was declined by TLR7 antagonist under the stimulation of a ligand, such as R848 (Resiquimod), for incubation of 20 hrs. The cell culture supernatant SEAP reporter activity was determined using QUANTI-Blue™ kit (Cat. #: rep-qbl, Invivogen, San Diego, Calif., USA) at a wavelength of 640 nm, a detection medium that turns purple or blue in the presence of alkaline phosphatase.

HEK293-Blue-hTLR7 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL A test compound in a serial dilution in the presence of final DMSO at 1% and 10 μL of 20 uM R848 in above DMEM, perform incubation under 37° C. in a CO₂ incubator for 20 hrs. Then 20 μL of the supernatant from each well was incubated with 180 μQuanti-blue substrate solution at 37° C. for 2 hrs and the absorbance was read at 620˜655 nm using a spectrophotometer. The signalling pathway that TLR7 activation leads to downstream NF-κB activation has been widely accepted, and therefore similar reporter assay was modified for evaluating TLR7 antagonist.

HEK293-Blue-hTLR-8 Cells Assay:

A stable HEK293-Blue-hTLR-8 cell line was purchased from InvivoGen (Cat. #: hkb-htlr8, San Diego, Calif., USA). These cells were originally designed for studying the stimulation of human TLR8 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR8 cells with TLR8 ligands. Therefore the reporter expression was declined by TLR8 antagonist under the stimulation of a ligand, such as R848, for incubation of 20 hrs. The cell culture supernatant SEAP reporter activity was determined using QUANTI-Blue™ kit (Cat #. rep-qbl, Invivogen, San Diego, Calif., USA) at a wavelength of 640 nm, a detection medium that turns purple or blue in the presence of alkaline phosphatase.

HEK293-Blue-hTLR8 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 60 μM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620˜655 nm using a spectrophotometer. The signalling pathway that TLR8 activation leads to downstream NF-KB activation has been widely accepted, and therefore similar reporter assay was modified for evaluating TLR8 antagonist.

HEK293-Blue-hTLR-9 Cells Assay:

A stable HEK293-Blue-hTLR-9 cell line was purchased from InvivoGen (Cat. #. hkb-htlr9, San Diego, Calif., USA). These cells were originally designed for studying the stimulation of human TLR9 by monitoring the activation of NF-κB. A SEAP (secreted embryonic alkaline phosphatase) reporter gene was placed under the control of the IFN-ρ minimal promoter fused to five NF-κB and AP-1-binding sites. The SEAP was induced by activating NF-κB and AP-1 via stimulating HEK-Blue hTLR9 cells with TLR9 ligands. Therefore the reporter expression was declined by TLR9 antagonist under the stimulation of a ligand, such as ODN2006 (Cat. #: tlrl-2006-1, Invivogen, San Diego, Calif., USA), for incubation of 20 hrs. The cell culture supernatant SEAP reporter activity was determined using QUANTI-Blue™ kit (Cat. #: rep-qbl, Invivogen, San Diego, Calif., USA) at a wavelength of 640 nm, a detection medium that turns purple or blue in the presence of alkaline phosphatase.

HEK293-Blue-hTLR9 cells were incubated at a density of 250,00018 450,000 cells/mL in a volume of 170 μL, in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM ODN2006 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620˜655 nm using a spectrophotometer. The signalling pathway that TLR9 activation leads to downstream NF-κB activation has been widely accepted, and therefore similar reporter assay was modified for evaluating TLR9 antagonist.

The compounds of formula (I) have human TLR7 and/or TLR8 inhibitory activities (IC₅₀ value) <0.5 μM, particularly <0.020 μM. Moreover, some compounds also have human TLR9 inhibitory activity <10 μM. Activity data of the compounds of the present invention were shown in Table 1.

TABLE 1
The activity of the compounds of present invention in
HEK293-BIue-hTLR-7/8/9 cells assays

| | TLR7 | TLR8 | TLR9 |
|---|---|---|---|
| Example | IC₅₀ (μM) | IC₅₀ (μM) | IC₅₀ (μM) |
| 1 | 0.005 | <0.003 | 9.7 |
| 2 | 0.052 | 0.006 | 51.8 |
| 2A | 0.041 | <0.003 | 41.0 |
| 2B | 0.015 | 0.008 | 34.6 |
| 3 | 0.012 | <0.003 | 81.9 |
| 4 | 0.006 | 0.01 | 11.4 |
| 5 | 0.007 | <0.003 | 15.5 |
| 6 | 0.031 | 0.026 | 29.8 |
| 7 | 0.063 | 0.035 | 68.5 |
| 8 | 0.004 | <0.003 | 10.3 |
| 9 | 0.014 | <0.003 | 10.9 |
| 10 | 0.36 | 0.029 | 18.4 |
| 11 | 0.051 | 0.062 | >100 |
| 12 | 0.15 | 0.005 | 6.3 |
| 13 | 0.29 | 0.052 | >100 |
| 14 | 0.037 | 0.007 | 29.4 |
| 15 | 0.03 | 0.015 | 14 |
| 16 | 0.074 | 0.008 | 39.9 |
| 17 | 0.14 | 0.025 | >100 |
| 18 | 0.005 | <0.003 | 54.8 |
| 19 | 0.014 | <0.003 | 50.7 |
| 20 | 0.002 | <0.003 | 14.9 |
| 21 | 0.071 | 0.009 | 38 |
| 22 | 0.039 | 0.019 | 37 |
| 23 | 0.007 | 0.004 | 7.0 |
| 24 | 0.015 | <0.003 | 23.7 |
| 25 | 0.012 | <0.003 | 12.4 |
| 26 | 0.023 | <0.003 | 46 |
| 27 | 0.06 | 0.021 | 28.1 |
| 28 | 0.20 | 0.086 | >100 |
| 29 | 0.037 | 0.014 | >100 |
| 30 | 0.37 | 0.13 | >100 |
| 31 | 0.005 | 0.009 | 9.2 |
| 32 | 0.078 | 0.020 | 3.7 |
| 33 | 0.002 | <0.003 | 23.6 |
| 34A | 0.004 | <0.003 | 8.5 |
| 34B | 0.007 | 0.006 | 10.0 |
| 35A | 0.028 | 0.006 | 42.7 |
| 35B | 0.065 | <0.003 | 50.2 |
| 36 | 0.13 | 0.025 | 12.3 |
| 37 | 0.002 | 0.006 | 7.8 |
| 38 | 0.003 | <0.003 | 6.0 |
| 39 | 0.007 | <0.003 | 35.8 |
| 40 | 0.015 | <0.003 | 22.4 |
| 41 | 0.015 | <0.003 | 23.2 |
| 42 | 0.005 | <0.003 | 10.6 |
| 43 | 0.010 | <0.003 | 12.3 |
| 44 | 0.018 | 0.043 | 15.7 |
| 45 | <0.003 | <0.003 | 11.6 |
| 46A | 0.008 | 0.016 | 23.0 |
| 46B | 0.021 | 0.055 | 15.3 |
| 47A | <0.003 | <0.003 | 10.1 |
| 47B | <0.003 | <0.003 | 10.5 |
| 48 | 0.004 | <0.003 | 11.7 |
| 49A | 0.025 | 0.022 | 10.7 |
| 49B | 0.020 | 0.009 | 7.3 |
| 49C | 0.008 | 0.005 | 7.7 |

### Example 51

The compounds with good solubility and high metabolic stability are considered to be desirable as they can provide a favorable in vivo PK profiles and thus sufficient exposure in the targeted tissues or organs. Compounds of present invention were tested in following assays to demonstrate above mentioned properties.

### LYSA Solubility Assay

LYSA study is used to determine the aqueous solubility of tested compounds. Samples are prepared in duplicate from 10 mM DMSO stock solution. After evaporation of DMSO with a centrifugal vacuum evaporator, compounds are dissolved in a 0.05 M phosphate buffer (pH 6.5), stirred for one hr and shaken for two hrs. After one night, the solutions are filtered using a microtiter filter plate. Then the filtrate and its 1/10 dilution are analyzed by HPLC-UV. In addition, a four-point calibration curve is prepared from the 10 mM stock solutions and used for the solubility determination of the compounds. The results are in μg/mL. In case the percentage of sample measured in solution after evaporation divided by the calculated maximum of sample amount is bigger than 80%, the solubility is reported as bigger than this value.

The compounds of present invention showed good solubility of >350 μg/mL determined in the above assay.

TABLE 2
Solubility of compounds
of present invention

| | LYSA |
|---|---|
| Example No | (μg/mL) |
| 2 | >585.0 |
| 2A | >580.0 |
| 2B | >625.0 |
| 3 | >690.0 |
| 4 | >660.0 |
| 5 | >655.0 |
| 6 | >640.0 |
| 7 | >605.0 |
| 8 | >680.0 |
| 9 | >685.0 |
| 10 | >655.0 |
| 11 | >740.0 |
| 12 | >665.0 |
| 13 | >725.0 |
| 14 | >695.0 |
| 16 | >450.0 |
| 17 | >485.0 |
| 18 | >530.0 |
| 19 | >510.0 |
| 20 | >515.0 |
| 21 | >435.0 |
| 22 | >455.0 |
| 23 | >315.0 |
| 24 | >475.0 |
| 25 | >470.0 |
| 26 | >520.0 |
| 27 | >585.0 |
| 28 | 480 |
| 29 | 565.0 |
| 30 | 375.0 |
| 31 | >597.0 |
| 32 | >640.0 |
| 33 | 350.0 |
| 34A | >620.0 |
| 34B | >610.0 |
| 35A | >600.0 |
| 35B | >625.0 |
| 36 | >595.0 |
| 37 | >660.0 |
| 38 | >650.0 |
| 39 | >625.0 |
| 40 | 555.0 |
| 41 | >675.0 |
| 42 | >550.0 |
| 43 | >560.0 |
| 44 | >600.0 |
| 45 | >660.0 |
| 46A | >650.0 |
| 46B | >667.0 |
| 47A | >620.0 |
| 47B | >605.0 |
| 48 | >625.0 |
| 49A | >660.0 |
| 49B | >690.0 |
| 49C | >710.0 |

### Example 52

Human Microsome Stability Assay

The human microsomal stability assay is used for early assessment of metabolic stability of a test compound in human liver microsomes.

Human liver microsomes (Cat. NO.: 452117, Corning, USA; Cat. NO.: H2610, Xenotech, USA) were preincubated with test compound for 10 minutes at 37° C. in 100 mM potassium phosphate buffer, pH 7.4. The reactions were initiated by adding NADPH regenerating system. The final incubation mixtures contained 1 μM test compound, 0.5 mg/mL liver microsomal protein, 1 mM MgCl₂, 1 mM NADP, 1 unit/mL isocitric dehydrogenase and 6 mM isocitric acid in 100 mM potassium phosphate buffer, pH 7.4. After incubation times of 0, 3, 6, 9, 15 and 30 minutes at 37° C., 300 μL of cold ACN (including internal standard) was added to 100 μL incubation mixture to terminate the reaction. Following precipitation and centrifugation, the amount of compound remaining in the samples were determined by LC-MS/MS. Controls of no NADPH regenerating system at zero and 30 minutes were also prepared and analyzed. The results were categorized as: low (<7.0 mL/min/kg), medium (7.0˜16.2 mL/min/kg) and high (16.2-23.2 mL/min/kg). The compounds of present invention showed good human liver microsome stability determined in the above assay.

TABLE 3
Human liver microsome stability of
the compounds of present invention

| | HLM |
|---|---|
| Example No | (mL/min/Kg) |
| 1 | 6.15 |
| 2 | 6.15 |
| 2A | 6.15 |
| 2B | 6.15 |
| 5 | 6.15 |
| 6 | 6.15 |
| 7 | 6.15 |
| 8 | 6.15 |
| 10 | 6.15 |
| 12 | 6.15 |
| 16 | 6.15 |
| 17 | 6.15 |
| 22 | 6.15 |
| 23 | 6.15 |
| 24 | 6.15 |
| 25 | 7.16 |
| 26 | 6.15 |
| 27 | 6.15 |
| 31 | 6.15 |
| 34A | 6.24 |
| 34B | 6.17 |
| 38 | 7.04 |
| 49B | 6.15 |
| 49C | 6.15 |

### Example 53

The compounds would be desirable to have minimal DDI liabilities. Therefore, the effects of compounds of formula (I) on major CYP isoforms, e.g. CYP2C9, CYP2D6 and CYP3A4, are determined.

### CYP Inhibition Assay

This is a high throughput screening assay used for assessment of reversible inhibition of CYP2C9, CYP2D6, and CYP3A4 activity of test compounds in human liver microsome (HLM) in early discovery stage.

TABLE 4
Chemicals and materials used in the CYP inhibition assay

| | | | | Final |
|---|---|---|---|---|
| | | | | Concentration |
| Substances | Description | Source | Cat. No. | in incubation |
| Human Liver Microsomes | | BD-Gentest | 452117 | 0.2 mg/mL |
| Diclofenac | CYP2C9 substrate | Sigma | D-6899 | 5 μM |
| 4′-Hydroxydiclofenac | CYP2C9 product | | | |
| 4′-OH-Diclofenac-13C6 | CYP2C9 internal | Becton | 451006 | |
| | standard | Dickinson | | |
| Dextromethorphan | CYP2D6 substrate | Sigma | D-2531 | 5 μM |
| Dextrorphan | CYP2D6 product | | | |
| Dextrorphan-D3 | CYP2D6 internal | Promochem | CERD-041 | |
| | standard | | | |
| Midazolam | CYP3A4 substrate | Roche | | 5 μM |
| 1′-Hydroxymidazolam | CYP3A4 product | | | |
| 1′-OH Midazolam-D4 | CYP3A4 internal | Roche | | |
| | standard | | | |
| Sulfaphenazole | CYP2C9 inhibitor | | | 2 μM |
| Quinidine | CYP2D6 inhibitor | | | 0.5 μM |
| Ketoconazole | CYP3A4 inhibitor | | | 0.5 μM |
| Glucose-6-phosphate | NADPH | Sigma | G-7250 | 3 mM |
| Glucose-6-Phosphate | regenerating | Roche | 1012761001 | 0.5 μL |
| Dehydrogenase | system components | Diagnostics | | |
| NADP | | Sigma | N-0505 | 1 mM |

### Procedure

10 mM DMSO stock solutions of test compounds were diluted in DMSO to generate 2 mM intermediate stock solution. 250 nL of intermediate stock solution were transferred in duplicate into 3 separate 384 well microtitre plates (assay-ready plates). A mixture of HLM and each substrate was made up. 45 μL of HLM substrate mix was then transferred to each well of an assay ready plate and mixed. The negative (solvent) and positive controls (standard inhibitor for each CYP) were included in each assay ready plate. The assay ready plate was warmed to 37° C. in an incubator over 10 minutes. 5 μL pre-warmed NADPH regenerating system was added to each incubation well to start the reaction. Final incubation volume was 50 μL. The assay plate then was placed back in the 37° C. incubator. After 5 minutes incubation (10 minutes for CYP2D6), incubates were quenched by addition of 50 μL quench reagent containing internal standards (400 ng/mL 13C6-4′-OH-Diclofenac, 20 ng/mL D3-Dextrorphan and 20 ng/mL D4-1′OH-Midazolam in acetonitrile). The supernatants were collected for RapidFire/MS/MS analysis.

RapidFire online solid phase extraction/sample injection system (Agilent) coupled with API4000 triple quadrupole mass spectrometer (AB Sciex) were used for sample analysis. The mobile phase composed of acetonitrile and water supplemented with 0.1% formic acid. A C4 solid phase extraction cartridge is used for sample separation. MS detection is achieved in positive ion MRM mode.

### Data Analysis

Peak areas for substrate, metabolite and internal standard are determined using the

RapidFire integrator software (version 3.6.12009.12296). Peak area ratios (PAR) of metabolite and internal standard (stable-labelled metabolite) are then calculated. The measurement window for each experiment is then defined:

PAR (0% activity)=average PAR for all incubations containing concentrated inhibitor;

Par (100% activity)=average PAR for all incubations containing no inhibitor (DMSO controls);

% Activity (test inhibitor) =[PAR(test inhibitor)-PAR(0% activity)][PAR(100% activity)-PAR(0% activity)];

% Inhibition (test inhibitor)=100-% Activity (test inhibitor).

Percentage inhibition data are then reported, in duplicate, for the 3 CYP enzymes. The results are categorized as:

>90%: potentially strong inhibitor (IC₅₀ expected <1 μM)

50-90%: likely weak to moderate inhibitor (IC₅₀ expected in 1-10 μM range) -30-50%: not or weak inhibitor

The compounds of present invention were found to have low CYP inhibition for CYP2C9, CYP2D6 and CYP3 A4 determined in the assays described above.

TABLE 5
CYP inhibition for CYP2C9,
CYP2D6 and CYP3A4

| | CYP inhibition |
|---|---|
| | % @10 uM |
| Example No | (2C9/2D6/3A4) |
| 2A | 26/9/5 |
| 3 | −1/16/−11 |
| 4 | −12/10/−14 |
| 5 | −1/16/−13 |
| 6 | −13/13/−10 |
| 8 | −8/10/−34 |
| 9 | 2/6/−18 |
| 10 | 11/26/10 |
| 11 | −4/5/−28 |
| 12 | 12/7/ND |
| 13 | 1/0/−22 |
| 14 | −2/−1/−24 |
| 15 | −17/−10/−18 |
| 16 | −8/5/2 |
| 17 | −4/3/−5 |
| 18 | −4/0/11 |
| 20 | −6/−12/6 |
| 23 | −16/35/ND |
| 24 | −20/−6/ND |
| 25 | 26/0/2 |
| 26 | −13/3/−5 |
| 27 | −8/6/−2 |
| 28 | 23/2/−30 |
| 30 | 20/20/20 |
| 31 | −1/2/−29 |
| 32 | −8/−22/−2 |
| 34A | −6/3/ND |
| 34B | −8/−10/−28 |
| 35A | 10/8/ND |
| 35B | 6/2/ND |
| 36 | 1/−10/ND |
| 37 | 4/3/ND |
| 38 | −3/−1/−2 |
| 39 | −7/5/5 |
| 42 | −4/−3/ND |
| 49A | −1/2/−25 |
| 49B | −2/−12/−36 |
| 49C | 11/8/ND |

ND : not detected

1. A compound of formula (I), 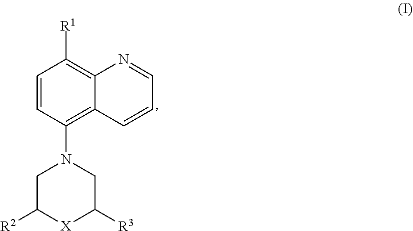
wherein
R¹ is halogen, C₁₋₆alkyl, haloC₁₋₆alkyl or C₂₋₆alkynyl;
R² is amino or —CONR⁴R⁵; wherein
R⁴ is H;
R⁵ is aminoC₁₋₆alkyl, heterocyclyl or heterocyclylC₁₋₆alkyl;
or R⁴ and R⁵ together with the nitrogen they are attached to form a heterocyclyl;
R³ is C₁₋₆alkyl;
X is O or CH₂;
or a pharmaceutically acceptable salt, enantiomer or diastereomer thereof.

2. A compound according to claim 1, wherein
R¹ is halogen, C₁₋₆alkyl, haloC₁₋₆alkyl or C₂₋₆alkynyl;
R² is amino or —CONR⁴R⁵; wherein
R⁴ is H;
R⁵ is (C₁₋₆alkylmorpholinyl)C₁₋₆alkyl, (C₁₋₆alkylpiperidinyl)C₁₋₆alkyl, aminoC₁₋₆alkyl, azabicyclo[2.2.1]heptanyl, azabicyclo[3.2.0]heptanyl, azabicyclo[3.2.1]octanyl, azabicyclo[3.3.1]nonanyl, azaspiro[2.4]heptanyl substituted by C₁₋₆alkyl, azepanyl, C₁₋₆alkylpiperidinyl, halopiperidinyl, halopyrrolidinyl, halopyrrolidinylC₁₋₆alkyl, morpholinylC₁₋₆alkyl, oxaazabicyclo[3.3.1]nonanyl or oxazepanyl;
or R⁴ and R⁵ together with the nitrogen they are attached to form diazaspiro[5.5]undecanyl, diazaspiro[4.4]nonanyl, azetidinyl, piperidinyl or pyrrolidinyl, said azetidinyl, piperidinyl and pyrrolidinyl being substituted by amino;
R³ is C₁₋₆alkyl;
X is 0 or CH₂;
or pharmaceutically acceptable salt, enantiomer or diastereomer thereof.

3. A compound according to claim 2, wherein R¹ is Br, Cl, I, CF₃, ethynyl or methyl.

4. A compound according to claim 3, wherein R¹ is Cl or CF₃.

5. A compound according to claim 3 or 4, wherein R² is —CONR⁴R⁵, wherein R⁴ is H; R⁵ is (C₁₋₆alkylmorpholinyl)C₁₋₆alkyl, (C₁₋₆alkylpiperidinyl)C₁₋₆alkyl, azabicyclo[3.2.1]octanyl, azabicyclo[3.3.1]nonanyl, azepanyl, C₁₋₆alkylpiperidinyl, morpholinylC₁₋₆alkyl or oxaazabicyclo [3.3.1] nonanyl.

6. A compound according to claim 5, wherein R² is —CONR⁴R⁵, wherein R⁴ is H; R⁵ is (methylmorpholinyl)methyl, (methylpiperidinyl)methyl, 3-azabicyclo[3.2.1]octan-8-yl, 8-azabicyclo[3.2.1]octan-3-yl, 9-azabicyclo[3.3.1]nonan-3-yl, 3-azabicyclo[3.3.1]nonan-7-yl, 3-azabicyclo[3.3.1]nonan-9-yl, azepan-4-yl, methylpiperidinyl, morpholinylmethyl, 3-oxa-7-azabicyclo[3.3.1]nonan-9-yl or 3-oxa-9-azabicyclo[3.3.1]nonan-7-yl.

7. A compound according to claim 6, wherein R⁵ is azabicyclo[3.2.1]octanyl or azabicyclo[3.3.1]nonanyl.

8. A compound according to claim 7, wherein R⁵ is 3-azabicyclo[3.2.1]octan-8-yl, 8-azabicyclo [3.2.]octan-3-yl, 9-azabicyclo [3.3.1]nonan-3-yl, 3-azabicyclo [3.3.1]nonan-7-yl or 3-azabicyclo [3.3.1] nonan-9-yl.

9. A compound according to claim 8, wherein X is O.

10. A compound according to claim 2, selected from:
(3R,55)-5-methyl-1-[8-(trifluoromethyl)-5-quinolyl]piperidin-3-amine;
cis-(2R,6R)-N-(4-fluoropyrrolidin-3-yl)-6-methyl-4-[8-(trifluoromethyl)-5-quinolyl]morpholine-2-carboxamide;
(2R,6R)-N-[(3R,4S)-4-fluoropyrrolidin-3-yl]-6-methyl-4-[8-(trifluoromethyl)-5-quinolyl]morpholine-2-carboxamide;
(2R,6R)-N-[(3S,4R)-4-fluoropyrrolidin-3-yl]-6-methyl-4-[8-(trifluoromethyl)-5-quinolyl] morpholine-2-carboxamide;
(2R,6R)-6-methyl-N-[(4-methylmorpholin-2-yl)methyl]-4-[8-(trifluoromethyl)-5-quinolyl] morpholine-2-carboxamide;
(2R,6R)-6-methyl-N-[(1-methyl-2-piperidyl)methyl]-4-[8-(trifluoromethyl)-5-quinolyl]morpholine-2-carboxamide;
(2R,6R)-6-methyl-N-(1-methyl-4-piperidyl)-4-[8-(trifluoromethyl)-5-quinolyl]morpholine-2-carboxamide;
(2R,6R)-N-(2-amino-2-methyl-propyl)-6-methyl-4-[8-(trifluoromethyl)-5-quinolyl] morpholine-2-carboxamide;
(3-aminoazetidin-1-yl)-[2R,6R)-6-methyl-4-[8-(trifluoromethyl)-5-quinolyl]morpholin-2-yl]methanone;
(2R,6R)-N-(azepan-4-yl)-6-methyl-4-[8-(trifluoromethyl)-5-quinolyl]morpholine-2-carboxamide;
(2R,6R)-6-methyl-N-(5-methyl-5-azaspiro[2.4]heptan-7-yl)-4-[8-(trifluoromethyl)-5-quinolyl] morpholine-2-carboxamide;
(3-aminopyrrolidin-1-yl)-[2R,6R)-6-methyl-4-[8-(trifluoromethyl)-5-quinolyl]morpholin-2-yl]methanone;
(2R,6R)-6-methyl-N-[(4-methylmorpholin-3-yl)methyl]-4-[8-(trifluoromethyl)-5-quinolyl] morpholine-2-carboxamide;
(4-amino-1-piperidyl)-[(2R,6R)-6-methyl-4-[8-(trifluoromethyl)-5-quinolyl]morpholin-2-yl]methanone;
(2R,6R)-6-methyl-N-(2-morpholinoethyl)-4-[8-(trifluoromethyl)-5-quinolyl]morpholine-2-carboxamide;
(2R,6R)-6-methyl-N-(1,4-oxazepan-6-yl)-4-[8-(trifluoromethyl)-5-quinolyl]morpholine-2-carboxamide;
(3R,55)-5-methyl-1-(8-methyl-5-quinolyl)piperidin-3-amine;
(2R,6R)-6-methyl-N-(1-methyl-4-piperidyl)-4-(8-methyl-5-quinolyl)morpholine-2-carboxamide;
(2R,6R)-6-methyl-N-[(4-methylmorpholin-2-yl)methyl]-4-(8-methyl-5-quinolyl)morpholine-2-carboxamide;
(2R,6R)-6-methyl-N-[(4-methylmorpholin-2-yl)methyl]-4-(8-nitro-5-quinolyl)morpholine -2-carboxamide;
cis-(2R,6R)-N-[4-fluoropyrrolidin-3-yl]-6-methyl-4-(8-nitro-5-quinolyl)morpholine-2-carboxamide;
(2R,6R)-6-methyl-N-(1-methyl-4-piperidyl)-4-(8-nitro-5-quinolyl)morpholine-2-carboxamide;
cis-(2R,6R)-N-[4-fluoropyrrolidin-3-yl]-6-methyl-4-(8-methyl-5-quinolyl)morpholine-2-carboxamide;
(2R,6R)-4-(8-chloro-5-quinolyl)-N-[(3S,4R)-4-fluoropyrrolidin-3-yl]-6-methyl-morpholine-2-carboxamide;
(3R,55)-1-(8-chloro-5-quinolyl)-5-methyl-piperidin-3-amine;
(2R,6R)-4-(8-chloro-5-quinolyl)-6-methyl-N-(1-methyl-4-piperidyl)morpholine-2-carboxamide;
(2R,6R)-N-(azepan-4-yl)-4-(8-chloro-5-quinolyl)-6-methyl-morpholine-2-carboxamide;
(2R,6R)-4-(8-chloro-5-quinolyl)-6-methyl-N-[(4-methylmorpholin-2-yl)methyl]morpholine-2-carboxamide;
(2R,6R)-N-[[(2S,4R)-4-fluoropyrrolidin-2-yl]methyl]-6-methyl-4-[8-(trifluoromethyl)-5-quinolyl]morpholine-2-carboxamide;
(2R,6R)-N-(5,5-difluoro-3-piperidyl)-6-methyl-4-[8-(trifluoromethyl)-5-quinolyl]morpholine-2-carboxamide;
(2R,6R)-N-[(4,4-difluoropyrrolidin-3-yl)methyl]-6-methyl-4-[8-(trifluoromethyl)-5-quinolyl] morpholine-2-carboxamide;
(2R,6R)-N-[[(25)-4,4-difluoropyrrolidin-2-yl]methyl]-6-methyl-4-[8-(trifluoromethyl)-5-quinolyl]morpholine-2-carboxamide;
(2R,6R)-N-(3-azabicyclo[3.2.1]octan-8-yl)-6-methyl-4-[8-(trifluoromethyl)-5-quinolyl]morpholine-2-carboxamide;
3,9-diazaspiro[5.5]undecan-3-yl-[(2R,6R)-6-methyl-4-[8-(trifluoromethyl)-5-quinolyl]morpholin-2-yl]methanone;
(2R,6R)-6-methyl-N-(3-oxa-9-azabicyclo[3.3.1]nonan-7-yl)-4-[8-(trifluoromethyl)-5-quinolyl]morpholine-2-carboxamide;
(2R,6R)-N-(9-azabicyclo[3.3.1]nonan-3-yl)-6-methyl-4-[8-(trifluoromethyl)-5-quinolyl]morpholine-2-carboxamide;
(2R,6R)-6-methyl-N-[1-(4-methylmorpholin-2-yl)ethyl]-4-[8-(trifluoromethyl)-5-quinolyl]morpholine-2-carboxamide;
2,7-diazaspiro[4.4]nonan-2-yl-[(2R,6R)-6-methyl-4-[8-(trifluoromethyl)-5-quinolyl]morpholin-2-yl]methanone;
(2R,6R)-N-(3-azabicyclo[3.3.1]nonan-7-yl)-6-methyl-4-[8-(trifluoromethyl)-5-quinolyl]morpholine-2-carboxamide;
(2R,6R)-4-(8-iodo-5-quinolyl)-6-methyl-N-(1-methyl-4-piperidyl)morpholine-2-carboxamide;
(2R,6R)-4-(8-iodo-5-quinolyl)-6-methyl-N-[(4-methylmorpholin-2-ylmethyl]morpholine-2-carboxamide;
cis-(2R,6R)-N-[4-fluoropyrrolidin-3-yl]-4-(8-iodo-5-quinolyl)-6-methyl-morpholine-2-carboxamide;
cis-(2R,6R)-4-(8-bromo-5-quinolyl)-N-[4-fluoropyrrolidin-3-yl]-6-methyl-morpholine-2-carboxamide;
(2R,6R)-4-(8-bromo-5-quinolyl)-6-methyl-N-(1-methyl-4-piperidyl)morpholine-2-carboxamide;
(2R,6R)-4-(8-ethynyl-5-quinolyl)-6-methyl-N-(1-methyl-4-piperidyl)morpholine-2-carboxamide;
(2R,6R)-N-[(1R,4R)-2-azabicyclo[2.2.1]heptan-5-yl]-6-methyl-4-[8-(trifluoromethyl)-5-quinolyl]morpholine-2-carboxamide;
(2R,6R)-N-(3-azabicyclo[3.3.1]nonan-9-yl)-6-methyl-4-[8-(trifluoromethyl)-5-quinolyl] morpholine-2-carboxamide;
(2R,6R)-6-methyl-N-(3-oxa-7-azabicyclo[3.3.1]nonan-9-yl)-4-[8-(trifluoromethyl)-5-quinolyl]morpholine-2-carboxamide;
(2R,6R)-N-(8-azabicyclo[3.2.1]octan-3-yl)-6-methyl-4-[8-(trifluoromethyl)-5-quinolyl]morpholine-2-carboxamide;
(2R,6R)-6-methyl-N-(morpholin-2-ylmethyl)-4-[8-(trifluoromethyl)-5-quinolyl]morpholine-2-carboxamide; and
(2R,6R)-N-(3-azabicyclo[3.2.0]heptan-6-yl)-6-methyl-4-[8-(trifluoromethyl)-5-quinolyl]morpholine-2-carboxamide;
or a pharmaceutically acceptable salt, enantiomer or diastereomer thereof.

11. A process for the preparation of a compound according to any one of claims 1 to 10 comprising any of the following steps:
a) the reaction of compound of formula (VIII), 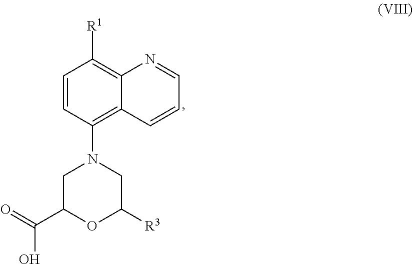
with amine (IX) in the presence of a coupling reagent;
b) the reaction of compound of formula (XII), 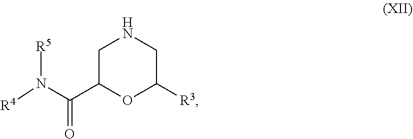
with compound of formula (VI) in the presence of a catalyst and a base;
c) the reaction of compound of formula (XIV), 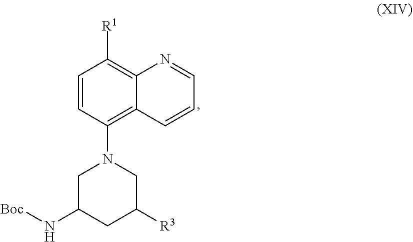
in the presence of an acid;
wherein the coupling reagent is HATU; the catalyst is Ruphos Pd-G2 and the base is Cs₂CO₃; the acid is TFA/CH₂Cl₂ or HCl in dioxane; R¹, R³, R⁴ and R⁵ are defined as in any one of claims 1 to 9.

12. A compound or pharmaceutically acceptable salt, enantiomer or diastereomer according to any one of claims 1 to 10 for use as therapeutically active substance.

13. A pharmaceutical composition comprising a compound in accordance with any one of claims 1 to 10 and a therapeutically inert carrier.

14. The use of a compound according to any one of claims 1 to 10 for the treatment or prophylaxis of systemic lupus erythematosus or lupus nephritis.

15. The use of a compound according to any one of claims 1 to 10 for the preparation of a medicament for the treatment or prophylaxis of systemic lupus erythematosus or lupus nephritis.

16. The use of a compound according to any one of claims 1 to 10 as the TLR7 or TLR8 or TLR9 antagonist.

17. The use of a compound according to any one of claims 1 to 10 as the TLR7 and TLR8 and TLR9 antagonist.

18. A compound or pharmaceutically acceptable salt, enantiomer or diastereomer according to any one of claims 1 to 10 for the treatment or prophylaxis of systemic lupus erythematosus or lupus nephritis.

19. A compound or pharmaceutically acceptable salt, enantiomer or diastereomer according to any one of claims 1 to 10, when manufactured according to a process of claim 11.

20. A method for the treatment or prophylaxis of systemic lupus erythematosus or lupus nephritis, which method comprises administering a therapeutically effective amount of a compound as defined in any one of claims 1 to 10.

21. The invention as hereinbefore described.
